SEQUENCE LISTING (25 sequences)

SEQ ID NO: 1 - protein, 10 aa, Artificial Sequence
  Description of Artificial Sequence Synthetic peptide
YVYVADVAAK

SEQ ID NO: 2 - protein, 17 aa, Artificial Sequence
  Description of Artificial Sequence Synthetic peptide
YEMFNDKSQRAPDDKMF

SEQ ID NO: 3 - protein, 9 aa, Artificial Sequence
  Description of Artificial Sequence Synthetic peptide
YEMFNDKSF

SEQ ID NO: 4 - protein, 11 aa, Artificial Sequence
  Description of Artificial Sequence Synthetic peptide
  MOD_RES (3)..(3): Pyrrolysine
  MOD_RES (11)..(11): Ile or Leu
HRXEIFSHDFX

SEQ ID NO: 5 - protein, 10 aa, Artificial Sequence
  Description of Artificial Sequence Synthetic peptide
  MOD_RES (2)..(2): Ile or Leu
  MOD_RES (5)..(5): Ile or Leu
  MOD_RES (7)..(7): Pyrrolysine
FXIEXFXESS

SEQ ID NO: 6 - protein, 10 aa, Artificial Sequence
  Description of Artificial Sequence Synthetic peptide
  MOD_RES (4)..(4): Pyrrolysine
NEIXREIREI

SEQ ID NO: 7 - protein, 27 aa, Artificial Sequence
  Description of Artificial Sequence Synthetic peptide
  MOD_RES (1)..(1): Ile or Leu
  MOD_RES (11)..(11): Ile or Leu
  MOD_RES (15)..(15): Selenocysteine
  MOD_RES (21)..(21): Ile or Leu
  MOD_RES (27)..(27): Ile or Leu
XFKSIFEMMSXDSSXIFLKSXFIEIFX

SEQ ID NO: 8 - protein, 13 aa, Artificial Sequence
  Description of Artificial Sequence Synthetic peptide
  MOD_RES (11)..(11): Pyrrolysine
KNFLENFIESXFI

SEQ ID NO: 9 - protein, 15 aa, Artificial Sequence
  Description of Artificial Sequence Synthetic peptide
  MOD_RES (2)..(2): Pyrrolysine
  MOD_RES (14)..(14): Ile or Leu
FXEIFNDKSLDKFXI

SEQ ID NO: 10 - protein, 16 aa, Artificial Sequence
  Description of Artificial Sequence Synthetic peptide
  MOD_RES (5)..(5): Pyrrolysine
  MOD_RES (16)..(16): Ile or Leu
QCEIXWAREFLKEIGX

SEQ ID NO: 11 - protein, 8 aa, Artificial Sequence
  Description of Artificial Sequence Synthetic peptide
  MOD_RES (4)..(4): Selenocysteine
FIEXHFWI

SEQ ID NO: 12 - protein, 12 aa, Artificial Sequence
  Description of Artificial Sequence Synthetic peptide
  MOD_RES (7)..(7): Ile or Leu
  MOD_RES (10)..(10): Selenocysteine
  MOD_RES (11)..(11): Ile or Leu
FEWRHRXTRXXR

SEQ ID NO: 13 - protein, 9 aa, Artificial Sequence
  Description of Artificial Sequence Synthetic peptide
  MOD_RES (4)..(4): Ile or Leu
  MOD_RES (5)..(5): Pyrrolysine
  MOD_RES (8)..(8): Ile or Leu
QIEXXEIXE

SEQ ID NO: 14 - protein, 9 aa, Artificial Sequence
  Description of Artificial Sequence Synthetic peptide
  MOD_RES (5)..(5): Pyrrolysine
QCEIXWARE

SEQ ID NO: 15 - protein, 14 aa, Artificial Sequence
  Description of Artificial Sequence Synthetic peptide
  MOD_RES (2)..(2): Ile or Leu
  MOD_RES (9)..(9): Pyrrolysine
  MOD_RES (11)..(11): Ile or Leu
FXELFISBXSXFIE

SEQ ID NO: 16 - protein, 11 aa, Artificial Sequence
  Description of Artificial Sequence Synthetic peptide
  MOD_RES (5)..(5): Pyrrolysine
  MOD_RES (9)..(9): Ile or Leu
IEFRXEIFXEF

SEQ ID NO: 17 - protein, 9 aa, Artificial Sequence
  Description of Artificial Sequence Synthetic peptide
  MOD_RES (5)..(5): Pyrrolysine
  MOD_RES (9)..(9): Ile or Leu
IEFRXEIFX

SEQ ID NO: 18 - protein, 9 aa, Artificial Sequence
  Description of Artificial Sequence Synthetic peptide
  MOD_RES (4)..(4): Pyrrolysine
  MOD_RES (8)..(8): Ile or Leu
EFRXEIFXE

SEQ ID NO: 19 - protein, 9 aa, Artificial Sequence
  Description of Artificial Sequence Synthetic peptide
  MOD_RES (3)..(3): Pyrrolysine
  MOD_RES (7)..(7): Ile or Leu
FRXEIFXEF

SEQ ID NO: 20 - protein, 18 aa, Artificial Sequence
  Description of Artificial Sequence Synthetic peptide
YEMFNDKSFQRAPDDKMF

SEQ ID NO: 21 - protein, 9 aa, Artificial Sequence
  Description of Artificial Sequence Synthetic peptide
  MOD_RES (6)..(6): Selenocysteine
  MOD_RES (7)..(7): Pyrrolysine
  MOD_RES (8)..(8): Pyrrolysine
FEGRKXXXI

SEQ ID NO: 22 - protein, 14 aa, Artificial Sequence
  Description of Artificial Sequence Synthetic peptide
  MOD_RES (2)..(2): Ile or Leu
  MOD_RES (5)..(5): Pyrrolysine
  MOD_RES (7)..(7): Ile or Leu
  MOD_RES (8)..(8): Pyrrolysine
  MOD_RES (10)..(10): Ile or Leu
  MOD_RES (14)..(14): Pyrrolysine
PXFIXEXXIXGEIX

SEQ ID NO: 23 - DNA, 2941 bp, Artificial Sequence
  Description of Artificial Sequence Synthetic polynucleotide
  modified_base (623)..(802): a, c, t, g, unknown or other
  modified_base (1463)..(1687): a, c, t, g, unknown or other
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagccgccaccatggccctgcctgtgacagccctgctgctgcctctggctctgctgctgcatgccgctagacccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcggctccggagccacgaacttctctctgttaaagcaagcaggagacgtggaagaaaaccccggtcccatggcattgcctgtcacggcactccttctcccgctggccctgcttctccacgcggcgcgacccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcgcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctag

SEQ ID NO: 24 - DNA, 3220 bp, Artificial Sequence
  Description of Artificial Sequence Synthetic polynucleotide
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagccgccaccatggccctgcctgtgacagccctgctgctgcctctggctctgctgctgcatgccgctagacccgaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcaaccccccggacgctgcgaggggacaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcggctccggagccacgaacttctctctgttaaagcaagcaggagacgtggaagaaaaccccggtcccatggcattgcctgtcacggcactccttctcccgctggccctgcttctccacgcggcgcgaccccagtcggtgacccagcttggcagccacgtctctgtctctgagggagccctggttctgctgaggtgcaactactcatcgtctgttccaccatatctcttctggtatgtgcaataccccaaccaaggactccagcttctcctgaagtacacaacaggggccaccctggttaaaggcatcaacggttttgaggctgaatttaagaagagtgaaacctccttccacctgacgaaaccctcagcccatatgagcgacgcggctgagtacttctgtgctgtgaccgtcacgggcaggagagcacttacttttgggagtggaacaagactccaagtgcaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcgcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctag

SEQ ID NO: 25 - DNA, 3187 bp, Artificial Sequence
  Description of Artificial Sequence Synthetic polynucleotide
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagccgccaccatggccctgcctgtgacagccctgctgctgcctctggctctgctgctgcatgccgctagacccggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagttaccgggagtacaacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcggctccggagccacgaacttctctctgttaaagcaagcaggagacgtggaagaaaaccccggtcccatggcattgcctgtcacggcactccttctcccgctggccctgcttctccacgcggcgcgaccccaaaagatagaacagaattccgaggccctgaacattcaggagggtaaaacggccaccctgacctgcaactatacaaactattctccagcatacttacagtggtaccgacaagatccaggaagaggccctgttttcttgctactcatacgtgaaaatgagaaagaaaaaaggaaagaaagactgaaggtcacctttgataccacccttaaacagagtttgtttcatatcacagcctcccagcctgcagactcagctacctacctctgtgctctaaatgccagactcatgtttggagatggaactcagctggtggtgaagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcgcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctag

(19) United States
(12) Patent Application Publication
(10) Pub. No.: US 2022/0154281 A1
(43) Pub. Date: May 19, 2022

(54) IDENTIFICATION OF NEOANTIGENS WITH MHC CLASS II MODEL

(71) Applicant: Gritstone bio, Inc., Emeryville, CA (US)

(72) Inventors: Thomas Francis Boucher, Boston, MA (US); Brendan Bulik-Sullivan, Cambridge, MA (US); Jennifer Busby, Burlington, MA (US); Minh Duc Cao, Belmont, MA (US); Matthew Joseph Davis, Scituate, MA (US); Lauren Elizabeth Young, Boston, MA (US); Joshua Michael Francis, Weymouth, MA (US); Christine Denise Palmer, Cambridge, MA (US); Mojca Skoberne, Cambridge, MA (US); Roman Yelensky, Newton, MA (US)

(21) Appl. No.: 17/436,367

(22) PCT Filed: Mar. 6, 2020
(86) PCT No.: PCT/US2020/021508

§ 371 Date: Sep. 3, 2021

Related U.S. Application Data
(60) Provisional application No. 62/814,801, filed on Mar. 6, 2019.
(60) Provisional application No. 62/826,822, filed on Mar. 29, 2019.

(51) Int. Cl. C12Q 1/6886; G01N 33/569; G01N 33/574; G16B 30/00; G16B 20/00; G16B 40/20

(52) U.S. Cl. CPC C12Q 1/6886; G01N 33/56977; G16B 40/20; G16B 30/00; G16B 20/00; G01N 33/57484

(57) ABSTRACT

A method for identifying T-cells that are antigen-specific for at least one neoantigen that is likely to be presented by class II MHC alleles on surfaces of tumor cells of a subject. Peptide sequences of tumor neoantigens are obtained by sequencing the tumor cells of the subject. The peptide sequences are input into a machine-learned presentation model to generate presentation likelihoods for the tumor neoantigens, each presentation likelihood representing the likelihood that a neoantigen is presented by the class II MHC alleles on the surfaces of the tumor cells of the subject. A subset of the neoantigens is selected based on the presentation likelihoods. T-cells that are antigen-specific for at least one of the neoantigens in the subset are identified. These T-cells can be expanded for use in T-cell therapy. TCRs of these identified T-cells can also be sequenced and cloned into new T-cells for use in T-cell therapy.

### BACKGROUND

Therapeutic vaccines and T-cell therapy based on tumor-specific neoantigens hold great promise as a next-generation of personalized cancer immunotherapy.¹⁻³ Cancers with a high mutational burden, such as non-small cell lung cancer (NSCLC) and melanoma, are particularly attractive targets of such therapy given the relatively greater likelihood of neoantigen generation.^{4,5} Early evidence shows that neoantigen-based vaccination can elicit T-cell responses⁶ and that neoantigen targeted T-cell therapy can cause tumor regression under certain circumstances in selected patients.⁷

In particular, identification of MHC class II-presented neoantigens for use in neoantigen-based vaccination and neoantigen targeted T-cell therapy is a promising treatment because up to 50% of neoantigen reactive TIL comprises CD4 cells, which respond to neoantigens presented by MHC class II alleles. These CD4 cells have been shown to assist CD8 cells in anti-tumor response, and in some instances to directly attack tumor cells. Despite this promising potential of MHC class II-presented neoantigens for use in cancer treatment, positive predictive values (PPV) for MHC class II-presented neoantigens are lower than PPVs for MHC class I-presented neoantigens that are recognized by CD8 cells.

These relatively worse presentation prediction outcomes for MHC class II-presented neoantigens may be due in part to the structure of MHC class I molecules relative to MHC class I molecules. Specifically, MHC class II molecules tend to have more open peptide binding grooves relative to MHC class I molecules. As a result of this difference in structure, MHC class I molecules tend to bind peptides of 8-11 amino acids in length, while MHC class II molecules bind peptides of more variable lengths (FIG. 14F). Due to the variability in the length of peptides presented by MHC class II molecules, the peptides presented by MHC class II molecules may be more difficult to predict relative to the peptides presented by MHC class I molecules.

Thus, identification of MHC class II-presented neoantigens and neoantigen-recognizing T-cells has become a central challenge in assessing tumor responses^{77,110}, examining tumor evolution¹¹¹ and designing the next generation of personalized therapies¹¹². Current neoantigen identification techniques are either time-consuming and laborious^{84,96}, or insufficiently precise^{87,91-93}. Furthermore, although it has recently been demonstrated that neoantigen-recognizing T-cells are a major component of TIL^{84,96,113,114} and circulate in the peripheral blood of cancer patients¹⁰⁷, current methods for identifying neoantigen-reactive T-cells have some combination of the following three limitations: (1) they rely on difficult-to-obtain clinical specimens such as TIL^{98,98} or leukaphereses¹⁰⁷ (2) they require screening impractically large libraries of peptides⁹⁵ or (3) they rely on MHC multimers, which may practically be available for only a small number of MHC alleles.

Furthermore, initial methods have been proposed incorporating mutation-based analysis using next-generation sequencing, RNA gene expression, and prediction of MHC binding affinity of candidate neoantigen peptides⁸. However, these proposed methods can fail to model the entirety of the epitope generation process, which contains many steps (e.g., TAP transport, proteasomal cleavage, MHC binding, transport of the peptide-MHC complex to the cell surface, and/or TCR recognition for MHC; endocytosis or autophagy, cleavage via extracellular or lysosomal proteases (e.g., cathepsins), and/or competition with the CLIP peptide for HLA-DM-catalyzed HLA binding) in addition to gene expression and MHC binding⁹. Consequently, existing methods are likely to suffer from reduced low positive predictive value (PPV). (FIG. 1A)

Indeed, analyses of peptides presented by tumor cells performed by multiple groups have shown that <5% of peptides that are predicted to be presented using gene expression and MHC binding affinity can be found on the tumor surface MHC^{10,11} (FIG. 1B). This low correlation between binding prediction and MHC presentation was further reinforced by recent observations of the lack of predictive accuracy improvement of binding-restricted neoantigens for checkpoint inhibitor response over the number of mutations alone.¹² These presentation prediction failings are particularly true in the case of neoantigens presented by MHC class II alleles.

This low positive predictive value (PPV) of existing methods for predicting presentation presents a problem for neoantigen-based vaccine design and for neoantigen-based T-cell therapy. If vaccines are designed using predictions with a low PPV, most patients are unlikely to receive a therapeutic neoantigen and fewer still are likely to receive more than one (even assuming all presented peptides are immunogenic). Similarly, if therapeutic T-cells are designed based on predictions with a low PPV, most patients are unlikely to receive T-cells that are reactive to tumor neoantigens and the time and physical resource cost of identifying predictive neoantigens using downstream laboratory techniques post-prediction may be unduly high. Thus, neoantigen vaccination and T-cell therapy with current methods is unlikely to succeed in a substantial number of subjects having tumors. (FIG. 1C)

Additionally, previous approaches generated candidate neoantigens using only cis-acting mutations, and largely neglected to consider additional sources of neo-ORFs, including mutations in splicing factors, which occur in multiple tumor types and lead to aberrant splicing of many genes¹³, and mutations that create or remove protease cleavage sites.

Finally, standard approaches to tumor genome and transcriptome analysis can miss somatic mutations that give rise to candidate neoantigens due to suboptimal conditions in library construction, exome and transcriptome capture, sequencing, or data analysis. Likewise, standard tumor analysis approaches can inadvertently promote sequence artifacts or germline polymorphisms as neoantigens, leading to inefficient use of vaccine capacity or auto-immunity risk, respectively.

### SUMMARY

Disclosed herein is an optimized approach for identifying and selecting neoantigens presented by MHC class II alleles for personalized cancer vaccines, for T-cell therapy, or both. First, optimized tumor exome and transcriptome analysis approaches for neoantigen candidate identification using next-generation sequencing (NGS) are addressed. These methods build on standard approaches for NGS tumor analysis to ensure that the highest sensitivity and specificity neoantigen candidates are advanced, across all classes of genomic alteration. Second, novel approaches for high-PPV MHC class II allele-presented neoantigen selection are presented to overcome the specificity problem and ensure that MHC class II allele-presented neoantigens advanced for vaccine inclusion and/or as targets for T-cell therapy are more likely to elicit anti-tumor immunity. These approaches include, depending on the embodiment, trained statistic regression or nonlinear deep learning MHC class II models that jointly model peptide-MHC class II allele mappings as well as the per-MHC class II allele motifs for peptides of multiple lengths, sharing statistical strength across peptides of different lengths. The nonlinear MHC class II deep learning models particularly can be designed and trained to treat different MHC alleles in the same cell as independent, thereby addressing problems with linear models that would have them interfere with each other. Finally, additional considerations for personalized vaccine design and manufacturing based on MHC class II allele-presented neoantigens, and for production of personalized MHC class II allele-presented neoantigen-specific T-cells for T-cell therapy, are addressed.

The model disclosed herein outperforms state-of-the-art predictors trained on binding affinity and early predictors based on MS peptide data by up to an order of magnitude. By more reliably predicting presentation of peptides by MHC class II alleles, the model enables more time- and cost-effective identification of MHC class II allele-presented neoantigen-specific or tumor antigen-specific T-cells for personalized therapy using a clinically practical process that uses limited volumes of patient peripheral blood, screens few peptides per patient, and does not necessarily rely on MHC multimers. However, in another embodiment, the model disclosed herein can be used to enable more time- and cost-effective identification of MHC class II allele-presented tumor antigen-specific T cells using MHC multimers, by decreasing the number of peptides bound to MHC multimers that need to be screened in order to identify MHC class II allele-presented neoantigen- or tumor antigen-specific T cells.

The predictive performance of the MHC class II model disclosed herein on the TIL neoepitope dataset and the prospective neoantigen-reactive T-cell identification task demonstrate that it is now possible to obtain therapeutically-useful MHC class II allele-presented neoepitope predictions by modeling MHC class II allele processing and presentation. In summary, this work offers practical in silico MHC class II allele-presented antigen identification for MHC class II allele-presented antigen-targeted immunotherapy, thereby accelerating progress towards cures for patients.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A shows current clinical approaches to neoantigen identification.

FIG. 1B shows that <5% of predicted bound peptides are presented on tumor cells.

FIG. 1C shows the impact of the neoantigen prediction specificity problem.

FIG. 1D shows that binding prediction is not sufficient for neoantigen identification.

FIG. 1E shows probability of MHC-I presentation as a function of peptide length.

FIG. 1F shows an example peptide spectrum generated from Promega's dynamic range standard.

FIG. 1G shows how the addition of features increases the model positive predictive value.

FIG. 2A is an overview of an environment for identifying likelihoods of peptide presentation in patients, in accordance with an embodiment.

FIGS. 2B and 2C illustrate a method of obtaining presentation information, in accordance with an embodiment.

FIG. 3 is a high-level block diagram illustrating the computer logic components of the presentation identification system, according to one embodiment.

FIG. 4 illustrates an example set of training data, according to one embodiment.

FIG. 5 illustrates an example network model in association with an MHC allele.

FIG. 6A illustrates an example network model NN_{H}(⋅) shared by MHC alleles, according to one embodiment.

FIG. 6B illustrates an example network model NN_{H}(⋅) shared by MHC alleles, according to another embodiment.

FIG. 7 illustrates generating a presentation likelihood for a peptide in association with an MHC allele using an example network model.

FIG. 8 illustrates generating a presentation likelihood for a peptide in association with a MHC allele using example network models.

FIG. 9 illustrates generating a presentation likelihood for a peptide in association with MHC alleles using example network models.

FIG. 10 illustrates generating a presentation likelihood for a peptide in association with MHC alleles using example network models.

FIG. 11 illustrates generating a presentation likelihood for a peptide in association with MHC alleles using example network models.

FIG. 12 illustrates generating a presentation likelihood for a peptide in association with MHC alleles using example network models.

FIG. 13A illustrates a sample frequency distribution of mutation burden in NSCLC patients.

FIG. 13B illustrates the number of presented neoantigens in simulated vaccines for patients selected based on an inclusion criteria of whether the patients satisfy a minimum mutation burden, in accordance with an embodiment.

FIG. 13C compares the number of presented neoantigens in simulated vaccines between selected patients associated with vaccines including treatment subsets identified based on presentation models and selected patients associated with vaccines including treatment subsets identified through current state-of-the-art models, in accordance with an embodiment.

FIG. 13D compares the number of presented neoantigens in simulated vaccines between selected patients associated with vaccines including treatment subsets identified based on a single per-allele presentation model for HLA-A*02:01 and selected patients associated with vaccines including treatment subsets identified based on both per-allele presentation models for HLA-A*02:01 and HLA-B*07:02. The vaccine capacity is set as v=20 epitopes, in accordance with an embodiment.

FIG. 13E compares the number of presented neoantigens in simulated vaccines between patients selected based on mutation burden and patients selected by expectation utility score, in accordance with an embodiment.

FIG. 14A is a histogram of lengths of peptides eluted from class II MHC alleles on human tumor cells and tumor infiltrating lymphocytes (TIL) using mass spectrometry.

FIG. 14B illustrates the dependency between mRNA quantification and presented peptides per residue for two example datasets.

FIG. 14C compares performance results for example presentation models trained and tested using two example datasets.

FIG. 14D is a histogram that depicts the quantity of peptides sequenced using mass spectrometry for each sample of a total of 73 samples comprising HLA class II molecules.

FIG. 14E is a histogram that depicts the quantity of samples in which a particular MHC class II molecule allele was identified.

FIG. 14F is a histogram that depicts the proportion of peptides presented by the MHC class II molecules in the 73 total samples, for each peptide length of a range of peptide lengths.

FIG. 14G is a line graph that depicts the relationship between gene expression and prevalence of presentation of the gene expression product by a MHC class II molecule, for genes present in the 73 samples.

FIG. 14H is a line graph that compares the performance of identical models with varying inputs, at predicting the likelihood that peptides in a testing dataset of peptides will be presented by a MHC class II molecule.

FIG. 14I is a line graph that compares the performance of three different models at predicting the likelihood that peptides in a testing dataset of peptides will be presented by a MHC class II molecule.

FIG. 14J depicts an exemplar embodiment of the Bi-LSTM model of FIG. 14I, configured to predict peptide presentation by HLA-DRB (a MHC class II gene).

FIG. 14K is a line graph that depicts full precision-recall curves for the Bi-LSTM, the MLP, the RNN, and the Binding Affinity models of FIG. 14I.

FIG. 14L is a line graph that compares the performance of a best-in-class prior art model using two different criteria and the presentation model disclosed herein with two different inputs, at predicting the likelihood that peptides in a testing dataset of peptides will be presented by a MHC class II molecule.

FIG. 14M is a histogram that depicts the quantity of peptides sequenced using mass spectrometry at a q-value of less than 0.1 for each sample of a total of 230 samples comprising human tumors (NSCLC, lymphoma, and ovarian cancer) and cell lines (EBV) including HLA class II molecules.

FIG. 14N is a histogram that depicts the quantity of samples in which a particular MHC class II molecule allele was identified.

FIG. 14O depicts a peptide bound to a MHC class I molecule and peptide bound to a MHC class II molecule.

FIG. 14P depicts an exemplar embodiment of an Inception neural network of the Inception model of FIG. 14Q, configured to predict peptide presentation by MHC class II molecules.

FIG. 14Q is a line graph that compares the performance of the “Bi-LSTM” and the “Inception” presentation models at predicting the likelihood that peptides in a testing dataset of peptides will be presented by at least one of the MHC class II molecules present in the testing dataset.

FIG. 15 compares the predictive performance of the “MS Model,” “NetMHCIIpan rank”: NetMHCIIpan 3.1⁷⁷, taking the lowest NetMHCIIpan percentile rank across HLA-DRB1*15:01 and HLA-DRB5*01:01, and “NetMHCIIpan nM”: NetMHCIIpan 3.1, taking the strongest affinity in nM units across HLA-DRB1*15:01 and HLA-DRB5*01:01, at ranking the peptides in the HLA-DRB1*15:01/HLA-DRB5*01:01 test dataset.

FIG. 16 depicts exemplary embodiments of TCR constructs for introducing a TCR into recipient cells.

FIG. 17 depicts an exemplary P526 construct backbone nucleotide sequence for cloning TCRs into expression systems for therapy development.

FIG. 18 depicts an exemplary construct sequence for cloning patient neoantigen-specific TCR, clonotype 1 TCR into expression systems for therapy development.

FIG. 19 depicts an exemplary construct sequence for cloning patient neoantigen-specific TCR, clonotype 3 into expression systems for therapy development.

FIG. 20 is a flow chart of a method for providing a customized, neoantigen-specific treatment to a patient, in accordance with an embodiment.

FIG. 21 illustrates an example computer for implementing the entities shown in FIGS. 1 and 3.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response.

As used herein the term “neoantigen” is an antigen that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358.

As used herein the term “tumor neoantigen” is a neoantigen present in a subject's tumor cell or tissue but not in the subject's corresponding normal cell or tissue.

As used herein the term “neoantigen-based vaccine” is a vaccine construct based on one or more neoantigens, e.g., a plurality of neoantigens.

As used herein the term “candidate neoantigen” is a mutation or other aberration giving rise to a new sequence that may represent a neoantigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T-cell receptor.

As used herein the term “immunogenic” is the ability to elicit an immune response. e.g., via T-cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “truncal mutation” is a mutation originating early in the development of a tumor and present in a substantial portion of the tumor's cells.

As used herein the term “subclonal mutation” is a mutation originating later in the development of a tumor and present in only a subset of the tumor's cells.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the tumor peptidome, meaning the union of the peptidomes of all cells that comprise the tumor).

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “MHC multimers” is a peptide-MHC complex comprising multiple peptide-MHC monomer units.

As used herein the term “MHC tetramers” is a peptide-MHC complex comprising four peptide-MHC monomer units.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T-cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include tumor type, tumor sub-type, and smoking history.

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Methods of Identifying Neoantigens

Disclosed herein are methods for identifying T-cells that are antigen-specific for neoantigens from tumor cells of a subject that are likely to be presented by class II MHC alleles on a surface of the tumor cells. The method includes obtaining exome, transcriptome, and/or whole genome nucleotide sequencing data from the tumor cells as well as normal cells of the subject. This nucleotide sequencing data is used to obtain a peptide sequence of each neoantigen in a set of neoantigens. The set of neoantigens is identified by comparing the nucleotide sequencing data from the tumor cells and the nucleotide sequencing data from the normal cells. Specifically, the peptide sequence of each neoantigen in the set of neoantigens comprises at least one alteration that makes it distinct from the corresponding wild-type peptide sequence identified from the normal cells of the subject. The method further includes encoding the peptide sequence of each neoantigen in the set of neoantigens into a corresponding numerical vector. Each numerical vector includes information describing the amino acids that make up the peptide sequence and the positions of the amino acids in the peptide sequence. The method further comprises inputting the numerical vectors into a machine-learned presentation model to generate a presentation likelihood for each neoantigen in the set of neoantigens. Each presentation likelihood represents the likelihood that the corresponding neoantigen is presented by the class II MHC alleles on the surface of the tumor cells of the subject. The machine-learned presentation model comprises a plurality of parameters and a function. The plurality of parameters are identified based on a training data set. The training data set comprises, for each sample in a plurality of samples, a label obtained by mass spectrometry measuring presence of peptides bound to at least one class II MHC allele in a set of class II MHC alleles identified as present in the sample, and training peptide sequences encoded as numerical vectors that include information describing the amino acids that make up the peptides and the positions of the amino acids in the peptides. The function represents a relation between the numerical vector received as input by the machine-learned presentation model and the presentation likelihood generated as output by the machine-learned presentation model based on the numerical vector and the plurality of parameters. The method further includes selecting a subset of the set of neoantigens, based on the presentation likelihoods, to generate a set of selected neoantigens. The method further comprises identifying T-cells that are antigen-specific for at least one of the neoantigens in the subset, and returning these identified T-cells.

In some embodiments, inputting the numerical vector into the machine-learned presentation model comprises applying the machine-learned presentation model to the peptide sequence of the neoantigen to generate a dependency score for each of the class II MHC alleles. The dependency score for an class II MHC allele indicates whether the class II MHC allele will present the neoantigen, based on the particular amino acids at the particular positions of the peptide sequence. In further embodiments, inputting the numerical vector into the machine-learned presentation model further comprises transforming the dependency scores to generate a corresponding per-allele likelihood for each class II MHC allele indicating a likelihood that the corresponding class II MHC allele will present the corresponding neoantigen, and combining the per-allele likelihoods to generate the presentation likelihood of the neoantigen. In some embodiments, transforming the dependency scores models the presentation of the neoantigen as mutually exclusive across the class II MHC alleles. In alternative embodiments, inputting the numerical vector into the machine-learned presentation model further comprises transforming a combination of the dependency scores to generate the presentation likelihood. In such embodiments, transforming the combination of the dependency scores models the presentation of the neoantigen as interfering between the class II MHC alleles.

In some embodiments, the set of presentation likelihoods are further identified by one or more allele noninteracting features. In such embodiments, the method further comprises applying the machine-learned presentation model to the allele noninteracting features to generate a dependency score for the allele noninteracting features. The dependency score indicates whether the peptide sequence of the corresponding neoantigen will be presented based on the allele noninteracting features. In some embodiments, the method further comprises combining the dependency score for each class II MHC allele with the dependency score for the allele noninteracting features, transforming the combined dependency score for each class II MHC allele to generate a per-allele likelihood for each class II MHC allele, and combining the per-allele likelihoods to generate the presentation likelihood. The per-allele likelihood for a class II MHC allele indicates a likelihood that the class II MHC allele will present the corresponding neoantigen. In alternative embodiments, the method further comprises combining the dependency scores for the class II MHC alleles and the dependency score for the allele noninteracting features, and transforming the combined dependency scores to generate the presentation likelihood.

In some embodiments, the class II MHC alleles include two or more different class II MHC alleles.

In some embodiments, the at least one class II MHC allele in the set of class II MHC alleles identified as present in the sample of the training data set includes two or more different types of class II MHC alleles.

In some embodiments, the peptide sequences comprise peptide sequences having lengths other than 9 amino acids.

In some embodiments, encoding the peptide sequence comprises encoding the peptide sequence using a one-hot encoding scheme.

In some embodiments, the plurality of samples comprise at least one of cell lines engineered to express a single class II MHC allele, cell lines engineered to express a plurality of class II MHC alleles, human cell lines obtained or derived from a plurality of patients, fresh or frozen tumor samples obtained from a plurality of patients, and fresh or frozen tissue samples obtained from a plurality of patients.

In some embodiments, the training data set further comprises at least one of data associated with peptide-MHC binding affinity measurements for at least one of the peptides, and data associated with peptide-MHC binding stability measurements for at least one of the peptides.

In some embodiments, the set of presentation likelihoods are further identified by expression levels of the class II MHC alleles in the subject, as measured by RNA-seq or mass spectrometry.

In some embodiments, the set of presentation likelihoods are further identified by features comprising at least one of predicted affinity between a neoantigen in the set of neoantigens and the class II MHC alleles, and predicted stability of the neoantigen encoded peptide-MHC complex.

In some embodiments, the set of numerical likelihoods are further identified by features comprising at least one of the C-terminal sequences flanking the neoantigen encoded peptide sequence within its source protein sequence, and the N-terminal sequences flanking the neoantigen encoded peptide sequence within its source protein sequence.

In some embodiments, selecting the set of selected neoantigens comprises selecting neoantigens that have an increased likelihood of being presented on the tumor cell surface relative to unselected neoantigens, based on the machine-learned presentation model.

In some embodiments, selecting the set of selected neoantigens comprises selecting neoantigens that have an increased likelihood of being capable of inducing a tumor-specific immune response in the subject relative to unselected neoantigens based on the machine-learned presentation model.

In some embodiments, selecting the set of selected neoantigens comprises selecting neoantigens that have an increased likelihood of being capable of being presented to naïve T-cells by professional antigen presenting cells (APCs) relative to unselected neoantigens, based on the presentation model. In such embodiments, the APC is optionally a dendritic cell (DC).

In some embodiments, selecting the set of selected neoantigens comprises selecting neoantigens that have a decreased likelihood of being subject to inhibition via central or peripheral tolerance relative to unselected neoantigens, based on the machine-learned presentation model.

In some embodiments, selecting the set of selected neoantigens comprises selecting neoantigens that have a decreased likelihood of being capable of inducing an autoimmune response to normal tissue in the subject relative to unselected neoantigens, based on the machine-learned presentation model.

In some embodiments, the one or more tumor cells are selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T-cell lymphocytic leukemia, non-small cell lung cancer, and small cell lung cancer.

In some embodiments, the method further comprises generating an output for constructing a personalized cancer vaccine from the set of selected neoantigens. In such embodiments, the output for the personalized cancer vaccine may comprise at least one peptide sequence or at least one nucleotide sequence encoding the set of selected neoantigens.

In some embodiments, the machine-learned presentation model is a neural network model. In such embodiments, the neural network model may include a plurality of network models for the class II MHC alleles, each network model assigned to a corresponding class II MHC allele of the class II MHC alleles and including a series of nodes arranged in one or more layers. In such embodiments, the neural network model may be trained by updating the parameters of the neural network model, the parameters of at least two network models being jointly updated for at least one training iteration.

In such embodiments, each network model can further include one or more convolutional neural networks, each of the one or more convolutional neural networks including a series of nodes arranged in one or more layers and having a filter of a different size. The filter of each of the one or more convolutional neural networks can be sized to identify the positions of the amino acids in the peptide sequence of each neoantigen that comprise a binding core or a binding anchor of the peptide sequence.

In some embodiments, the machine-learned presentation model may be a deep learning model that includes one or more layers of nodes.

In some embodiments, identifying the T-cells comprises co-culturing the T-cells with one or more of the neoantigens in the subset under conditions that expand the T-cells.

In some embodiments, identifying the T-cells comprises contacting the T-cells with an MHC multimer comprising one or more of the neoantigens in the subset under conditions that allow binding between the T-cells and the MHC multimer.

In some embodiments, the method further comprises identifying T-cell receptors (TCR) of the identified T-cells. In such embodiments, identifying the T-cell receptors may comprise sequencing the T-cell receptor sequences of the identified T-cells. In such embodiments, the method may further comprise genetically engineering T-cells to express at least one of the one or more identified T-cell receptors, culturing the T-cells under conditions that expand the T-cells, and infusing the expanded T-cells into the subject. In such embodiments, genetically engineering the T-cells to express at least one of the identified T-cell receptors may comprise cloning the T-cell receptor sequences of the identified T-cells into an expression vector, and transfecting each of the T-cells with the expression vector.

In some embodiments, the method further comprises culturing the identified T-cells under conditions that expand the identified T-cells, and infusing the expanded T-cells into the subject.

Disclosed herein is also an isolated T-cell that is antigen-specific for at least one selected neoantigen in the subset of neoantigens described above.

International Patent Publication No. WO 2018/195357 and International Patent Publication No. WO 2019/050994 are hereby incorporated by reference in their entireties. International Patent Publication No. WO 2018/195357 describes methods for predicting antigen presentation by MHC class II molecules. International Patent Publication No. WO 2019/050994 describes methods for identification of T-cells that are antigen-specific to antigens presented by MHC molecules. While these publications are referred to in this section of the application, the disclosures provided in International Patent Publication Nos. WO 2018/195357 and WO 2019/050994 are hereby incorporated by reference in their entireties in every section of this application.

### III. Identification of Tumor Specific Mutations in Neoantigens

Also disclosed herein are methods for the identification of certain mutations (e.g., the variants or alleles that are present in cancer cells). In particular, these mutations can be present in the genome, transcriptome, proteome, or exome of cancer cells of a subject having cancer but not in normal tissue from the subject.

Genetic mutations in tumors can be considered useful for the immunological targeting of tumors if they lead to changes in the amino acid sequence of a protein exclusively in the tumor. Useful mutations include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence. Mutations can also include one or more of nonframeshift indel, missense or nonsense substitution, splice site alteration, genomic rearrangement or gene fusion, or any genomic or expression alteration giving rise to a neoORF.

Peptides with mutations or mutated polypeptides arising from for example, splice-site, frameshift, readthrough, or gene fusion mutations in tumor cells can be identified by sequencing DNA, RNA or protein in tumor versus normal cells.

Also mutations can include previously identified tumor specific mutations. Known tumor mutations can be found at the Catalogue of Somatic Mutations in Cancer (COSMIC) database.

A variety of methods are available for detecting the presence of a particular mutation or allele in an individual's DNA or RNA. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling-circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet. P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet. P. et al. can be a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they utilize incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

A number of initiatives obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to identify mutations. As described above, four major sequencing-by-synthesis platforms are currently available: the Genome Sequencers from Roche/454 Life Sciences, the IG Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., US Patent Application No. 2006/0252077) can be linked to each fragment to be captured on a surface coated with a respective second member of that coupling pair.

Subsequent to the capture, the sequence can be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies.

Any cell type or tissue can be utilized to obtain nucleic acid samples for use in methods described herein. For example, a DNA or RNA sample can be obtained from a tumor or a bodily fluid, e.g., blood, obtained by known techniques (e.g. venipuncture) or saliva. Alternatively, nucleic acid tests can be performed on dry samples (e.g. hair or skin). In addition, a sample can be obtained for sequencing from a tumor and another sample can be obtained from normal tissue for sequencing where the normal tissue is of the same tissue type as the tumor. A sample can be obtained for sequencing from a tumor and another sample can be obtained from normal tissue for sequencing where the normal tissue is of a distinct tissue type relative to the tumor.

Tumors can include one or more of lung cancer, melanoma, breast cancer, ovarian cancer, prostate cancer, kidney cancer, gastric cancer, colon cancer, testicular cancer, head and neck cancer, pancreatic cancer, brain cancer. B-cell lymphoma, acute myelogenous leukemia, chronic myelogenous leukemia, chronic lymphocytic leukemia, and T-cell lymphocytic leukemia, non-small cell lung cancer, and small cell lung cancer.

Alternatively, protein mass spectrometry can be used to identify or validate the presence of mutated peptides bound to MHC proteins on tumor cells. Peptides can be acid-eluted from tumor cells or from HLA molecules that are immunoprecipitated from tumor, and then identified using mass spectrometry.

### IV. Neoantigens

Neoantigens can include nucleotides or polypeptides. For example, a neoantigen can be an RNA sequence that encodes for a polypeptide sequence. Neoantigens useful in vaccines can therefore include nucleotide sequences or polypeptide sequences.

Disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

One or more polypeptides encoded by a neoantigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more neoantigens can be presented on the surface of a tumor.

One or more neoantigens can be is immunogenic in a subject having a tumor, e.g., capable of eliciting a T-cell response or a B cell response in the subject.

One or more neoantigens that induce an autoimmune response in a subject can be excluded from consideration in the context of vaccine generation for a subject having a tumor.

The size of at least one neoantigenic peptide molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the neoantigenic peptide molecules are equal to or less than 50 amino acids.

Neoantigenic peptides and polypeptides can be: for MHC Class I 15 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would consist of: (3) the entire stretch of novel tumor-specific amino acids-thus bypassing the need for computational or in vitro test-based selection of the strongest HLA-presented shorter peptide. In both cases, use of a longer peptide allows endogenous processing by patient-cells and may lead to more effective antigen presentation and induction of T-cell responses.

Neoantigenic peptides and polypeptides can be presented on an HLA protein. In some aspects neoantigenic peptides and polypeptides are presented on an HLA protein with greater affinity than a wild-type peptide. In some aspects, a neoantigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, neoantigenic peptides and polypeptides do not induce an autoimmune response and/or invoke immunological tolerance when administered to a subject.

Also provided are compositions comprising at least two or more neoantigenic peptides. In some embodiments the composition contains at least two distinct peptides. At least two distinct peptides can be derived from the same polypeptide. By distinct polypeptides is meant that the peptide vary by length, amino acid sequence, or both. The peptides are derived from any polypeptide known to or have been found to contain a tumor specific mutation. Suitable polypeptides from which the neoantigenic peptides can be derived can be found for example in the COSMIC database. COSMIC curates comprehensive information on somatic mutations in human cancer. The peptide contains the tumor specific mutation. In some aspects the tumor specific mutation is a driver mutation for a particular cancer type.

Neoantigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T-cell. For instance, neoantigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn. Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to induce CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

A neoantigenic peptide can be linked to the T helper peptide either directly or via a spacer either at the amino or carboxy terminus of the peptide. The amino terminus of either the neoantigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect a neoantigen includes a nucleic acid (e.g. polynucleotide) that encodes a neoantigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothiate backbone, or combinations thereof and it may or may not contain introns. A still further aspect provides an expression vector capable of expressing a polypeptide or portion thereof. Expression vectors for different-cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning. A Laboratory Manual, Cold Spring Harbor Laboratory, Cold Spring Harbor, N.Y.

### IV. Vaccine Compositions

Also disclosed herein is an immunogenic composition, e.g., a vaccine composition, capable of raising a specific immune response. e.g., a tumor-specific immune response. Vaccine compositions typically comprise a plurality of neoantigens, e.g., selected using a method described herein. Vaccine compositions can also be referred to as vaccines.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 neoantigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different neoantigen sequences, 6, 7, 8, 9, 10 11, 12, 13, or 14 different neoantigen sequences, or 12, 13 or 14 different neoantigen sequences.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or MHC class II molecules. Hence, vaccine compositions can comprise different fragments capable of associating with at least 2 preferred, at least 3 preferred, or at least 4 preferred MHC class I molecules and/or MHC class II molecules.

The vaccine composition can be capable of raising a specific cytotoxic T-cells response and/or a specific helper T-cell response.

A vaccine composition can further comprise an adjuvant and/or a carrier. Examples of useful adjuvants and carriers are given herein below. A composition can be associated with a carrier such as e.g. a protein or an antigen-presenting cell such as e.g. a dendritic cell (DC) capable of presenting the peptide to a T-cell.

Adjuvants are any substance whose admixture into a vaccine composition increases or otherwise modifies the immune response to a neoantigen. Carriers can be scaffold structures, for example a polypeptide or a polysaccharide, to which a neoantigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A. Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M. et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diptheria toxoid are suitable carriers. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Neoantigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-29), or lentivirus, including but not limited to second, third or hybrid second/third generation lentivirus and recombinant lentivirus of any generation designed to target specific cell types or receptors (See. e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more neoantigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T-cell receptor repertoires, Science. (2016) 352 (6291):1337-41, Lu et al., Efficient identification of mutated cancer antigens recognized by T-cells associated with durable tumor regressions, Clin Cancer Res. (2014) 20(13):3401-10). Upon introduction into a host, infected cells express the neoantigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of neoantigens, e.g., Salmonella typhi vectors, and the like will be apparent to those skilled in the art from the description herein.

### IV.A. Additional Considerations for Vaccine Design and Manufacture

### IV.A.1. Determination of a Set of Peptides that Cover all Tumor Subclones

Truncal peptides, meaning those presented by all or most tumor subclones, will be prioritized for inclusion into the vaccine.⁵³ Optionally, if there are no truncal peptides predicted to be presented and immunogenic with high probability, or if the number of truncal peptides predicted to be presented and immunogenic with high probability is small enough that additional non-truncal peptides can be included in the vaccine, then further peptides can be prioritized by estimating the number and identity of tumor subclones and choosing peptides so as to maximize the number of tumor subclones covered by the vaccine.⁵⁴

### IV.A.2. Neoantigen Prioritization

After all of the above neoantigen filters are applied, more candidate neoantigens may still be available for vaccine inclusion than the vaccine technology can support. Additionally, uncertainty about various aspects of the neoantigen analysis may remain and tradeoffs may exist between different properties of candidate vaccine neoantigens. Thus, in place of predetermined filters at each step of the selection process, an integrated multi-dimensional model can be considered that places candidate neoantigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of neoantigens may lower the probability that a tumor will escape immune attack via downregulation or mutation of HLA molecules)
7. Coverage of HLA classes (covering both HLA-I and HLA-II may increase the probability of therapeutic response and decrease the probability of tumor escape)

Additionally, optionally, neoantigens can be deprioritized (e.g., excluded) from the vaccination if they are predicted to be presented by HLA alleles lost or inactivated in either all or part of the patient's tumor. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017. Van Loo et al., 2010).

### V. Therapeutic and Manufacturing Methods

Also provided is a method of inducing a tumor specific immune response in a subject, vaccinating against a tumor, treating and or alleviating a symptom of cancer in a subject by administering to the subject one or more neoantigens such as a plurality of neoantigens identified using methods disclosed herein.

In some aspects, a subject has been diagnosed with cancer or is at risk of developing cancer. A subject can be a human, dog, cat, horse or any animal in which a tumor specific immune response is desired. A tumor can be any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T-cell lymphocytic leukemia, and B cell lymphomas.

A neoantigen can be administered in an amount sufficient to induce a CTL response.

A neoantigen can be administered alone or in combination with other therapeutic agents. The therapeutic agent is for example, a chemotherapeutic agent, radiation, or immunotherapy. Any suitable therapeutic treatment for a particular cancer can be administered.

In addition, a subject can be further administered an anti-immunosuppressive/immunostimulatory agent such as a checkpoint inhibitor. For example, the subject can be further administered an anti-CTLA antibody or anti-PD-1 or anti-PD-L1. Blockade of CTLA4 or PD-L1 by antibodies can enhance the immune response to cancerous cells in the patient. In particular, CTLA-4 blockade has been shown effective when following a vaccination protocol.

The optimum amount of each neoantigen to be included in a vaccine composition and the optimum dosing regimen can be determined. For example, a neoantigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of neoantigens present in the composition is/are tissue, cancer, and/or patient-specific. For instance, the exact selection of peptides can be guided by expression patterns of the parent proteins in a given tissue. The selection can be dependent on the specific type of cancer, the status of the disease, earlier treatment regimens, the immune status of the patient, and, of course, the HLA-haplotype of the patient. Furthermore, a vaccine can contain individualized components, according to personal needs of the particular patient. Examples include varying the selection of neoantigens according to the expression of the neoantigen in the particular patient or adjustments for secondary treatments following a first round or scheme of treatment.

For a composition to be used as a vaccine for cancer, neoantigens with similar normal self-peptides that are expressed in high amounts in normal tissues can be avoided or be present in low amounts in a composition described herein. On the other hand, if it is known that the tumor of a patient expresses high amounts of a certain neoantigen, the respective pharmaceutical composition for treatment of this cancer can be present in high amounts and/or more than one neoantigen specific for this particularly neoantigen or pathway of this neoantigen can be included.

Compositions comprising a neoantigen can be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when the cancer has metastasized. In such cases, in view of the minimization of extraneous substances and the relative nontoxic nature of a neoantigen, it is possible and can be felt desirable by the treating physician to administer substantial excesses of these compositions.

For therapeutic use, administration can begin at the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered at the site of surgical excision to induce a local immune response to the tumor. Disclosed herein are compositions for parenteral administration which comprise a solution of the neoantigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Neoantigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the neoantigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired neoantigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Felgner et al., Proc. Natl. Acad. Sci. USA 84: 7413-7414 (1987).

Neoantigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-29), or lentivirus, including but not limited to second, third or hybrid second/third generation lentivirus and recombinant lentivirus of any generation designed to target specific cell types or receptors (See. e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J Virol. (1998) 72 (12): 9873-9880). Dependent on the packaging capacity of the above mentioned viral vector-based vaccine platforms, this approach can deliver one or more nucleotide sequences that encode one or more neoantigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T-cell receptor repertoires, Science. (2016) 352 (6291):1337-41, Lu et al., Efficient identification of mutated cancer antigens recognized by T-cells associated with durable tumor regressions, Clin Cancer Res. (2014) 20(13):3401-10). Upon introduction into a host, infected cells express the neoantigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of neoantigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a tumor vaccine, comprising performing the steps of a method disclosed herein; and producing a tumor vaccine comprising a plurality of neoantigens or a subset of the plurality of neoantigens.

Neoantigens disclosed herein can be manufactured using methods known in the art. For example, a method of producing a neoantigen or a vector (e.g., a vector including at least one sequence encoding one or more neoantigens) disclosed herein can include culturing a host cell under conditions suitable for expressing the neoantigen or vector wherein the host cell comprises at least one polynucleotide encoding the neoantigen or vector, and purifying the neoantigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes a neoantigen or vector disclosed herein, optionally wherein the isolated polynucleotide further comprises a promoter sequence operably linked to the at least one nucleic acid sequence that encodes the neoantigen or vector. In certain embodiments the isolated polynucleotide can be cDNA.

### VI. Neoantigen Identification

### VI.A. Neoantigen Candidate Identification

Research methods for NGS analysis of tumor and normal exome and transcriptomes have been described and applied in the neoantigen identification space.^{6,14,15} The example below considers certain optimizations for greater sensitivity and specificity for neoantigen identification in the clinical setting. These optimizations can be grouped into two areas, those related to laboratory processes and those related to the NGS data analysis.

### VI.A.1. Laboratory Process Optimizations

The process improvements presented here address challenges in high-accuracy neoantigen discovery from clinical specimens with low tumor content and small volumes by extending concepts developed for reliable cancer driver gene assessment in targeted cancer panels¹⁶ to the whole-exome and -transcriptome setting necessary for neoantigen identification. Specifically, these improvements include:
1. Targeting deep (>500×) unique average coverage across the tumor exome to detect mutations present at low mutant allele frequency due to either low tumor content or subclonal state.
2. Targeting uniform coverage across the tumor exome, with <5% of bases covered at <100×, so that the fewest possible neoantigens are missed, by, for instance:
   a. Employing DNA-based capture probes with individual probe QC¹⁷
   b. Including additional baits for poorly covered regions
3. Targeting uniform coverage across the normal exome, where <5% of bases are covered at <20× so that the fewest neoantigens possible remain unclassified for somatic/germline status (and thus not usable as TSNAs)
4. To minimize the total amount of sequencing required, sequence capture probes will be designed for coding regions of genes only, as non-coding RNA cannot give rise to neoantigens. Additional optimizations include:
   a. supplementary probes for HLA genes, which are GC-rich and poorly captured by standard exome sequencing¹⁸
   b. exclusion of genes predicted to generate few or no candidate neoantigens, due to factors such as insufficient expression, suboptimal digestion by the proteasome, or unusual sequence features.
5. Tumor RNA will likewise be sequenced at high depth (>100M reads) in order to enable variant detection, quantification of gene and splice-variant (“isoform”) expression, and fusion detection. RNA from FFPE samples will be extracted using probe-based enrichment¹⁹, with the same or similar probes used to capture exomes in DNA.

### VI.A.2. NGS Data Analysis Optimizations

Improvements in analysis methods address the suboptimal sensitivity and specificity of common research mutation calling approaches, and specifically consider customizations relevant for neoantigen identification in the clinical setting. These include:
1. Using the HG38 reference human genome or a later version for alignment, as it contains multiple MHC regions assemblies better reflective of population polymorphism, in contrast to previous genome releases.
2. Overcoming the limitations of single variant callers²⁰ by merging results from different programs'
   a. Single-nucleotide variants and indels will be detected from tumor DNA, tumor RNA and normal DNA with a suite of tools including: programs based on comparisons of tumor and normal DNA, such as Strelka²¹ and Mutect²²; and programs that incorporate tumor DNA, tumor RNA and normal DNA, such as UNCeqR, which is particularly advantageous in low-purity samples²³.
   b. Indels will be determined with programs that perform local re-assembly, such as Strelka and ABRA²⁴.
   c. Structural rearrangements will be determined using dedicated tools such as Pindel²⁵ or Breakseq²⁶.
3. In order to detect and prevent sample swaps, variant calls from samples for the same patient will be compared at a chosen number of polymorphic sites.
4. Extensive filtering of artefactual calls will be performed, for instance, by:
   a. Removal of variants found in normal DNA, potentially with relaxed detection parameters in cases of low coverage, and with a permissive proximity criterion in case of indels
   b. Removal of variants due to low mapping quality or low base quality²⁷.
   c. Removal of variants stemming from recurrent sequencing artifacts, even if not observed in the corresponding normal²⁷. Examples include variants primarily detected on one strand.
   d. Removal of variants detected in an unrelated set of controls²⁷
5. Accurate HLA calling from normal exome using one of seq2HLA²⁸, ATHLATES²⁹ or Optitype and also combining exome and RNA sequencing data²⁸. Additional potential optimizations include the adoption of a dedicated assay for HLA typing such as long-read DNA sequencing³⁰, or the adaptation of a method for joining RNA fragments to retain continuity³¹
6. Robust detection of neo-ORFs arising from tumor-specific splice variants will be performed by assembling transcripts from RNA-seq data using CLASS³², Bayesembler³³, StringTie³⁴ or a similar program in its reference-guided mode (i.e., using known transcript structures rather than attempting to recreate transcripts in their entirety from each experiment). While Cufflinks “is commonly used for this purpose, it frequently produces implausibly large numbers of splice variants, many of them far shorter than the full-length gene, and can fail to recover simple positive controls. Coding sequences and nonsense-mediated decay potential will be determined with tools such as SpliceR³⁶ and MAMBA³⁷, with mutant sequences re-introduced. Gene expression will be determined with a tool such as Cufflinks” or Express (Roberts and Pachter, 2013). Wild-type and mutant-specific expression counts and/or relative levels will be determined with tools developed for these purposes, such as ASE³⁸ or HTSeq³⁹. Potential filtering steps include:
   a. Removal of candidate neo-ORFs deemed to be insufficiently expressed.
   b. Removal of candidate neo-ORFs predicted to trigger non-sense mediated decay (NMD).
7. Candidate neoantigens observed only in RNA (e.g., neoORFs) that cannot directly be verified as tumor-specific will be categorized as likely tumor-specific according to additional parameters, for instance by considering:
   a. Presence of supporting tumor DNA-only cis-acting frameshift or splice-site mutations
   b. Presence of corroborating tumor DNA-only trans-acting mutation in a splicing factor. For instance, in three independently published experiments with R625-mutant SF3B1, the genes exhibiting the most differentially splicing were concordant even though one experiment examined uveal melanoma patients⁴⁰, the second a uveal melanoma cell line⁴¹, and the third breast cancer patients⁴².
   c. For novel splicing isoforms, presence of corroborating “novel” splice-junction reads in the RNASeq data.
   d. For novel re-arrangements, presence of corroborating juxta-exon reads in tumor DNA that are absent from normal DNA
   e. Absence from gene expression compendium such as GTEx⁴³ (i.e. making germline origin less likely)
8. Complementing the reference genome alignment-based analysis by comparing assembled DNA tumor and normal reads (or k-mers from such reads) directly to avoid alignment and annotation based errors and artifacts. (e.g. for somatic variants arising near germline variants or repeat-context indels)

In samples with poly-adenylated RNA, the presence of viral and microbial RNA in the RNA-seq data will be assessed using RNA CoMPASS⁴⁴ or a similar method, toward the identification of additional factors that may predict patient response.

### VI.B. Isolation and Detection of HLA Peptides

Isolation of HLA-peptide molecules was performed using classic immunoprecipitation (IP) methods after lysis and solubilization of the tissue sample⁵⁵⁻⁵⁸. A clarified lysate was used for HLA specific IP.

Immunoprecipitation was performed using antibodies coupled to beads where the antibody is specific for HLA molecules. For a pan-Class I HLA immunoprecipitation, a pan-Class I CR antibody is used, for Class II HLA-DR, an HLA-DR antibody is used. Antibody is covalently attached to NHS-sepharose beads during overnight incubation. After covalent attachment, the beads were washed and aliquoted for IP.⁵⁹⁻⁶⁰ Immunoprecipitations can also be performed with antibodies that are not covalently attached to beads. Typically this is done using sepharose or magnetic beads coated with Protein A and/or Protein G to hold the antibody to the column. Some antibodies that can be used to selectively enrich MHC/peptide complex are listed below.

| Antibody Name | Specificity |
|---|---|
| W6/32 | Class I HLA-A, B, C |
| L243 | Class II-HLA-DR |
| Tu36 | Class II-HLA-DR |
| LN3 | Class II-HLA-DR |
| Tu39 | Class II-HLA-DR, DP, DQ |

The clarified tissue lysate is added to the antibody beads for the immunoprecipitation. After immunoprecipitation, the beads are removed from the lysate and the lysate stored for additional experiments, including additional IPs. The IP beads are washed to remove non-specific binding and the HLA/peptide complex is eluted from the beads using standard techniques. The protein components are removed from the peptides using a molecular weight spin column or C18 fractionation. The resultant peptides are taken to dryness by SpeedVac evaporation and in some instances are stored at −20 C prior to MS analysis.

Dried peptides are reconstituted in an HPLC buffer suitable for reverse phase chromatography and loaded onto a C-18 microcapillary HPLC column for gradient elution in a Fusion Lumos mass spectrometer (Thermo). MS1 spectra of peptide mass/charge (m/z) were collected in the Orbitrap detector at high resolution followed by MS2 low resolution scans collected in the ion trap detector after HCD fragmentation of the selected ion. Additionally, MS2 spectra can be obtained using either CID or ETD fragmentation methods or any combination of the three techniques to attain greater amino acid coverage of the peptide. MS2 spectra can also be measured with high resolution mass accuracy in the Orbitrap detector.

MS2 spectra from each analysis are searched against a protein database using Comet^{61,62} and the peptide identification are scored using Percolator⁶³⁻⁶⁵. Additional sequencing is performed using PEAKS studio (Bioinformatics Solutions Inc.) and other search engines or sequencing methods can be used including spectral matching and de novo sequencing⁷⁵.

### VI.B.1. MS Limit of Detection Studies in Support of Comprehensive HLA Peptide Sequencing

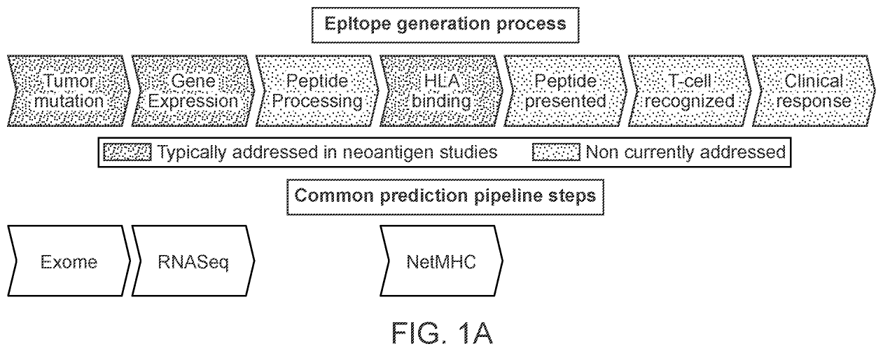

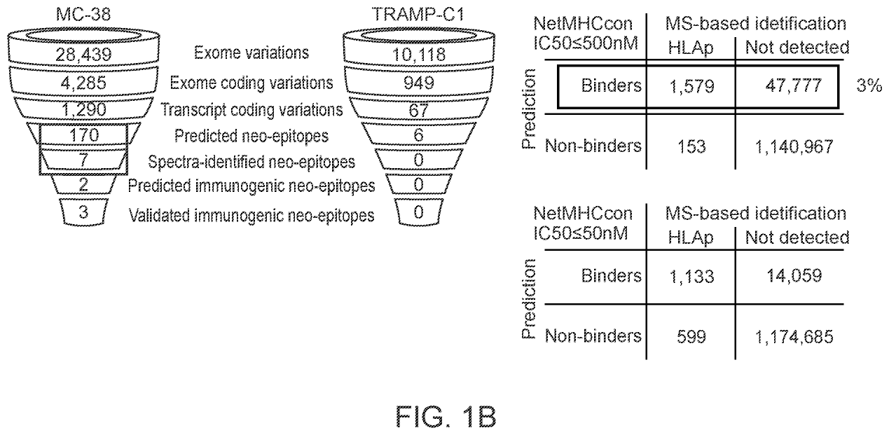

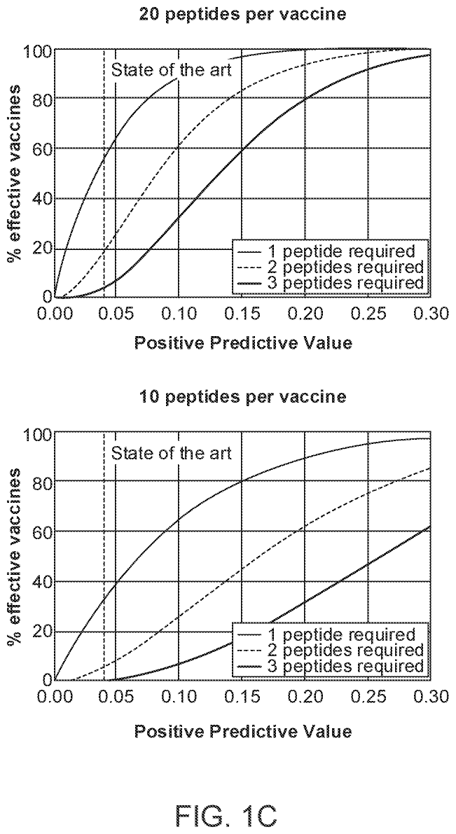

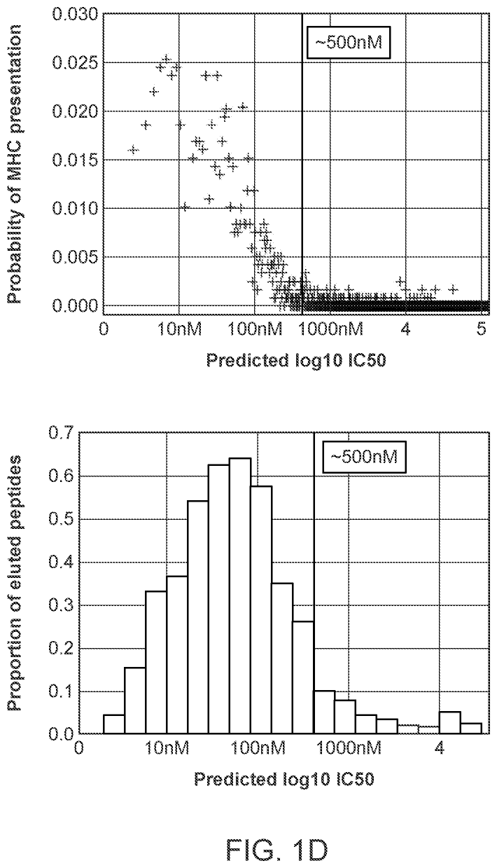

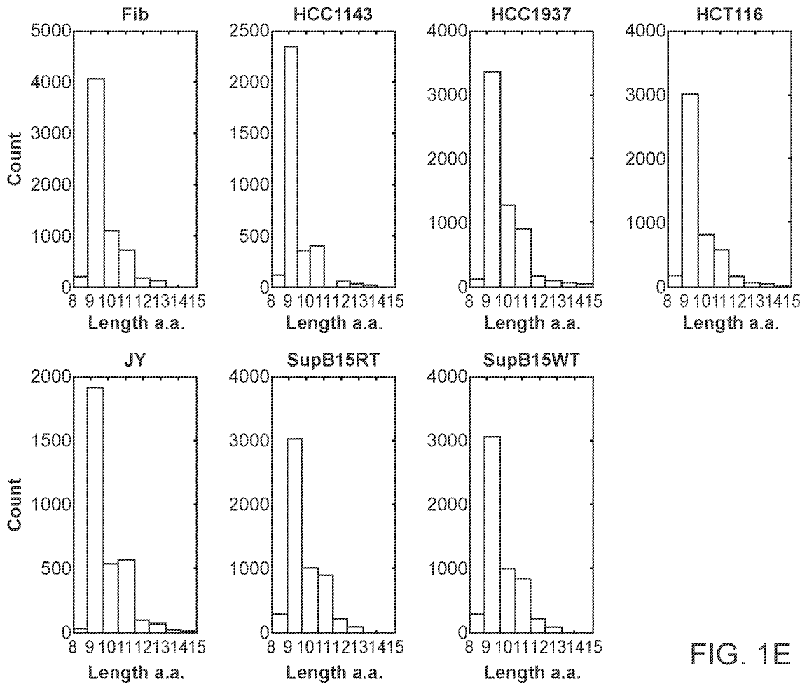

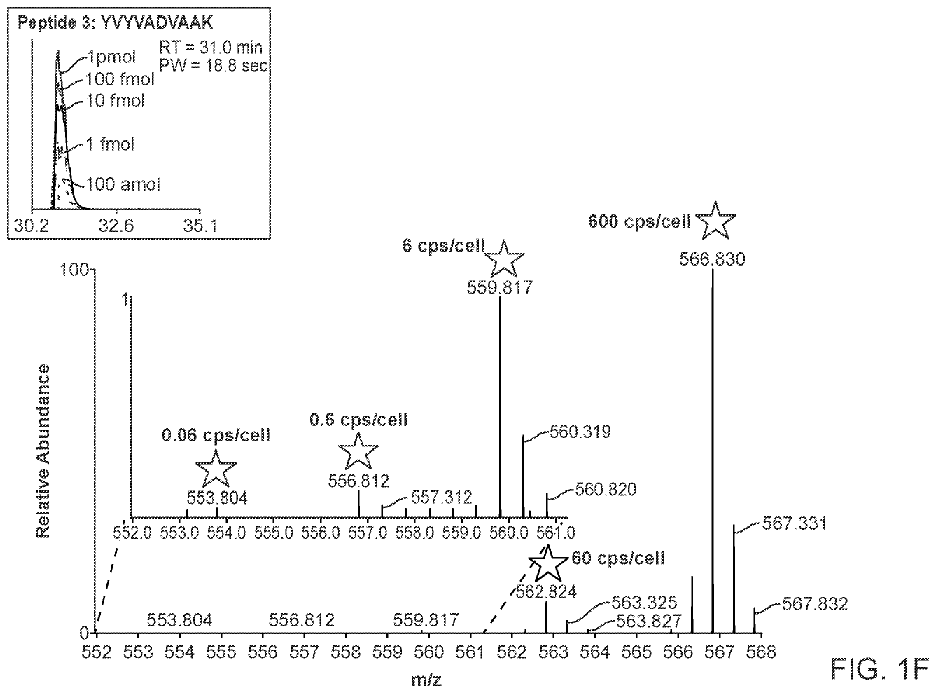

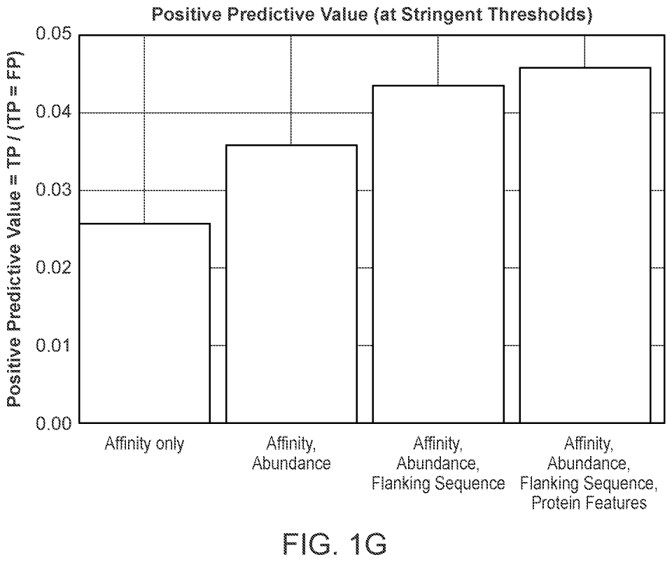

Using the peptide YVYVADVAAK it was determined what the limits of detection are using different amounts of peptide loaded onto the LC column. The amounts of peptide tested were 1 pmol, 100 fmol, 10 fmol, 1 fmol, and 100 amol. (Table 1) The results are shown in FIG. 1F. These results indicate that the lowest limit of detection (LoD) is in the attomol range (10⁻¹⁸), that the dynamic range spans five orders of magnitude, and that the signal to noise appears sufficient for sequencing at low femtomol ranges (10⁻¹⁵).

| Peptide m/z | Loaded on Column | | Copies/Cell in 1e9cells |
|---|---|---|---|
| 566.830 | 1 | pmol | 600 |
| 562.823 | 100 | fmol | 60 |
| 559.816 | 10 | fmol | 6 |
| 556.810 | 1 | fmol | 0.6 |
| 553.802 | 100 | amol | 0.06 |

### VII. Presentation Model

### VII.A. System Overview

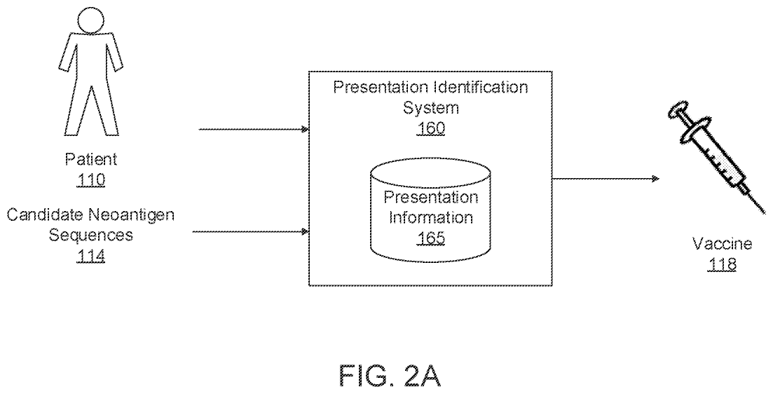

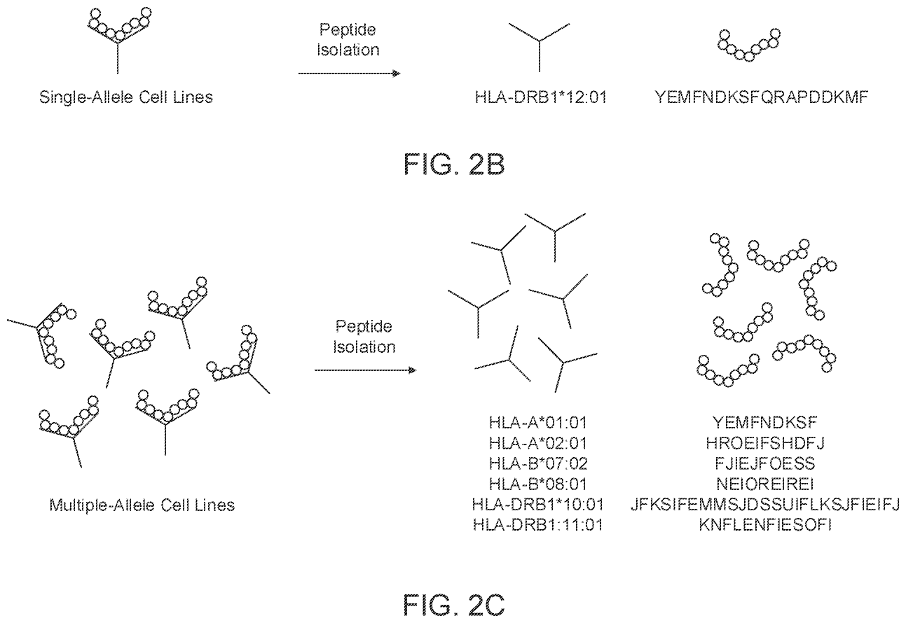

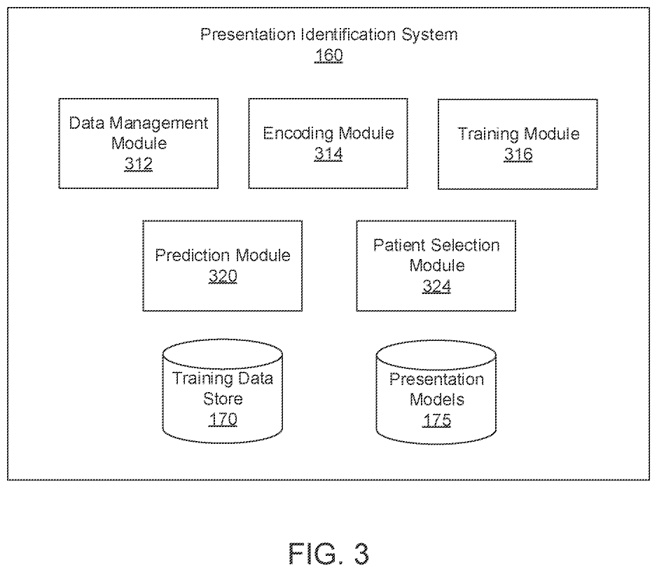

FIG. 2A is an overview of an environment 100 for identifying likelihoods of peptide presentation in patients, in accordance with an embodiment. The environment 100 provides context in order to introduce a presentation identification system 160, itself including a presentation information store 165.

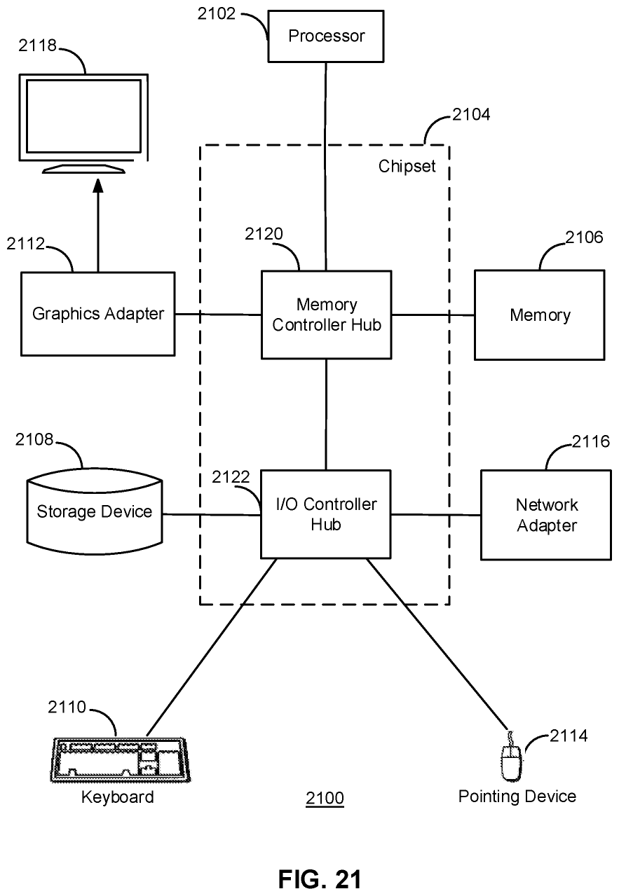

The presentation identification system 160 is one or computer models, embodied in a computing system as discussed below with respect to FIG. 21, that receives peptide sequences associated with a set of MHC alleles and determines likelihoods that the peptide sequences will be presented by one or more of the set of associated MHC alleles. The presentation identification system 160 may be applied to both class I and class II MHC alleles. This is useful in a variety of contexts. One specific use case for the presentation identification system 160 is that it is able to receive nucleotide sequences of candidate neoantigens associated with a set of MHC alleles from tumor cells of a patient 110 and determine likelihoods that the candidate neoantigens will be presented by one or more of the associated MHC alleles of the tumor and/or induce immunogenic responses in the immune system of the patient 110. Those candidate neoantigens with high likelihoods as determined by system 160 can be selected for inclusion in a vaccine 118, such an anti-tumor immune response can be elicited from the immune system of the patient 110 providing the tumor cells. Additionally, T-cells with TCRs that are responsive to candidate neoantigens with high presentation likelihoods can be produced for use in T-cell therapy, thereby also eliciting an anti-tumor immune response from the immune system of the patient 110.

The presentation identification system 160 determines presentation likelihoods through one or more presentation models. Specifically, the presentation models generate likelihoods of whether given peptide sequences will be presented for a set of associated MHC alleles, and are generated based on presentation information stored in store 165. For example, the presentation models may generate likelihoods of whether a peptide sequence “YVYVADVAAK” will be presented for the set of alleles HLA-A*02:01, HLA-A*03:01, HLA-B*07:02. HLA-B*08:03, HLA-C*01:04 on the cell surface of the sample. The presentation information 165 contains information on whether peptides bind to different types of MHC alleles such that those peptides are presented by MHC alleles, which in the models is determined depending on positions of amino acids in the peptide sequences. The presentation model can predict whether an unrecognized peptide sequence will be presented in association with an associated set of MHC alleles based on the presentation information 165. As previously mentioned, the presentation models may be applied to both class I and class II MHC alleles.

### VII.B. Presentation Information

FIG. 2 illustrates a method of obtaining presentation information, in accordance with an embodiment. The presentation information 165 includes two general categories of information: allele-interacting information and allele-noninteracting information. Allele-interacting information includes information that influence presentation of peptide sequences that are dependent on the type of MHC allele. Allele-noninteracting information includes information that influence presentation of peptide sequences that are independent on the type of MHC allele.

### VII.B.1. Allele-Interacting Information

Allele-interacting information primarily includes identified peptide sequences that are known to have been presented by one or more identified MHC molecules from humans, mice, etc. Notably, this may or may not include data obtained from tumor samples. The presented peptide sequences may be identified from cells that express a single MHC allele. In this case the presented peptide sequences are generally collected from single-allele cell lines that are engineered to express a predetermined MHC allele and that are subsequently exposed to synthetic protein. Peptides presented on the MHC allele are isolated by techniques such as acid-elution and identified through mass spectrometry. FIG. 2B shows an example of this, where the example peptide YEMFNDKSQRAPDDKMF, presented on the predetermined MHC allele HLA-DRB1*12:01, is isolated and identified through mass spectrometry. Since in this situation peptides are identified through cells engineered to express a single predetermined MHC protein, the direct association between a presented peptide and the MHC protein to which it was bound to is definitively known.

The presented peptide sequences may also be collected from cells that express multiple MHC alleles. Typically in humans, 6 different types of MHC-I and up to 12 different types of MHC-II molecules are expressed for a cell. Such presented peptide sequences may be identified from multiple-allele cell lines that are engineered to express multiple predetermined MHC alleles. Such presented peptide sequences may also be identified from tissue samples, either from normal tissue samples or tumor tissue samples. In this case particularly, the MHC molecules can be immunoprecipitated from normal or tumor tissue. Peptides presented on the multiple MHC alleles can similarly be isolated by techniques such as acid-elution and identified through mass spectrometry. FIG. 2C shows an example of this, where the six example peptides, YEMFNDKSF, HROEIFSHDFJ, FJIEJFOESS, NEIOREIREI, JFKSIFEMMSJDSSUIFLKSJFIEIFJ, and KNFLENFIESOFI, are presented on identified class I MHC alleles HLA-A*01:01, HLA-A*02:01, HLA-B*07:02, HLA-B*08:01, and class II MHC alleles HLA-DRB1*10:01, HLA-DRB1:11:01 and are isolated and identified through mass spectrometry. In contrast to single-allele cell lines, the direct association between a presented peptide and the MHC protein to which it was bound to may be unknown since the bound peptides are isolated from the MHC molecules before being identified.

Allele-interacting information can also include mass spectrometry ion current which depends on both the concentration of peptide-MHC molecule complexes, and the ionization efficiency of peptides. The ionization efficiency varies from peptide to peptide in a sequence-dependent manner. Generally, ionization efficiency varies from peptide to peptide over approximately two orders of magnitude, while the concentration of peptide-MHC complexes varies over a larger range than that.

Allele-interacting information can also include measurements or predictions of binding affinity between a given MHC allele and a given peptide. (72, 73, 74) One or more affinity models can generate such predictions. For example, going back to the example shown in FIG. 1D, presentation information 165 may include a binding affinity prediction of 1000 nM between the peptide YEMFNDKSF and the class I allele HLA-A*01:01. Few peptides with IC50>1000 nm are presented by the MHC, and lower IC50 values increase the probability of presentation. Presentation information 165 may include a binding affinity prediction between the peptide KNFLENFIESOFI and the class II allele HLA-DRB1:11:01.

Allele-interacting information can also include measurements or predictions of stability of the MHC complex. One or more stability models that can generate such predictions. More stable peptide-MHC complexes (i.e., complexes with longer half-lives) are more likely to be presented at high copy number on tumor cells and on antigen-presenting cells that encounter vaccine antigen. For example, going back to the example shown in FIG. 2C, presentation information 165 may include a stability prediction of a half-life of 1 h for the class I molecule HLA-A*01:01. Presentation information 165 may also include a stability prediction of a half-life for the class II molecule HLA-DRB11:11:01.

Allele-interacting information can also include the measured or predicted rate of the formation reaction for the peptide-MHC complex. Complexes that form at a higher rate are more likely to be presented on the cell surface at high concentration.

Allele-interacting information can also include the sequence and length of the peptide. MHC class I molecules typically prefer to present peptides with lengths between 8 and 15 peptides. 60-80% of presented peptides have length 9. MHC class II molecules typically prefer to present peptides with lengths between 6-30 peptides.

Allele-interacting information can also include the presence of kinase sequence motifs on the neoantigen encoded peptide, and the absence or presence of specific post-translational modifications on the neoantigen encoded peptide. The presence of kinase motifs affects the probability of post-translational modification, which may enhance or interfere with MHC binding.

Allele-interacting information can also include the expression or activity levels of proteins involved in the process of post-translational modification, e.g., kinases (as measured or predicted from RNA seq, mass spectrometry, or other methods).

Allele-interacting information can also include the probability of presentation of peptides with similar sequence in cells from other individuals expressing the particular MHC allele as assessed by mass-spectrometry proteomics or other means.

Allele-interacting information can also include the expression levels of the particular MHC allele in the individual in question (e.g. as measured by RNA-seq or mass spectrometry). Peptides that bind most strongly to an MHC allele that is expressed at high levels are more likely to be presented than peptides that bind most strongly to an MHC allele that is expressed at a low level.

Allele-interacting information can also include the overall neoantigen encoded peptide-sequence-independent probability of presentation by the particular MHC allele in other individuals who express the particular MHC allele.

Allele-interacting information can also include the overall peptide-sequence-independent probability of presentation by MHC alleles in the same family of molecules (e.g., HLA-A, HLA-B, HLA-C, HLA-DQ, HLA-DR, HLA-DP) in other individuals. For example, HLA-C molecules are typically expressed at lower levels than HLA-A or HLA-B molecules, and consequently, presentation of a peptide by HLA-C is a priori less probable than presentation by HLA-A or HLA-B. For another example, HLA-DP is typically expressed at lower levels than HLA-DR or HLA-DQ; consequently, presentation of a peptide by HLA-DP is a prior less probable than presentation by HLA-DR or HLA-DQ.

Allele-interacting information can also include the protein sequence of the particular MHC allele.

Any MHC allele-noninteracting information listed in the below section can also be modeled as an MHC allele-interacting information.

### VII.B.2. Allele-Noninteracting Information

Allele-noninteracting information can include C-terminal sequences flanking the neoantigen encoded peptide within its source protein sequence. For MHC-I, C-terminal flanking sequences may impact proteasomal processing of peptides. However, the C-terminal flanking sequence is cleaved from the peptide by the proteasome before the peptide is transported to the endoplasmic reticulum and encounters MHC alleles on the surfaces of cells. Consequently, MHC molecules receive no information about the C-terminal flanking sequence, and thus, the effect of the C-terminal flanking sequence cannot vary depending on MHC allele type. For example, going back to the example shown in FIG. 2C, presentation information 165 may include the C-terminal flanking sequence FOEIFNDKSLDKFJI of the presented peptide FJIEJFOESS identified from the source protein of the peptide.

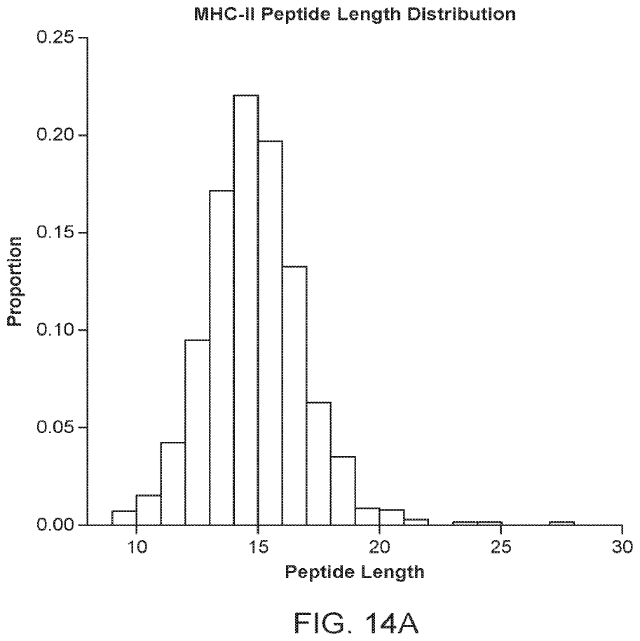

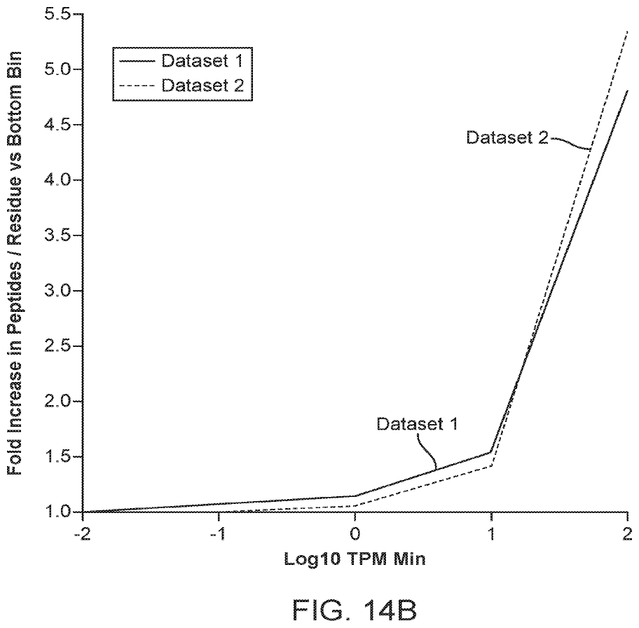

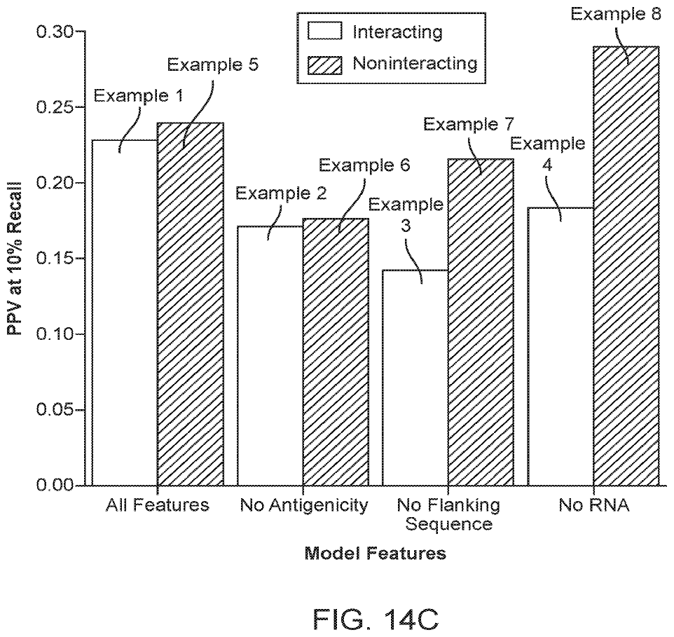

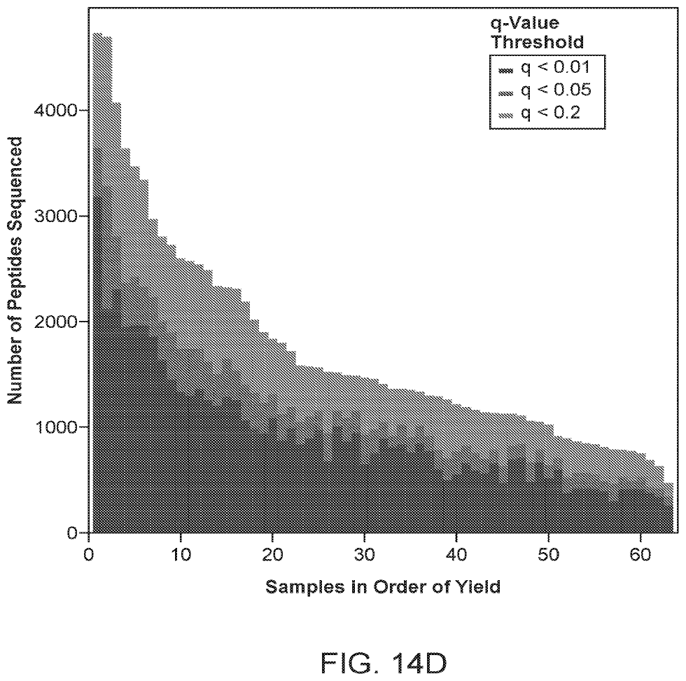

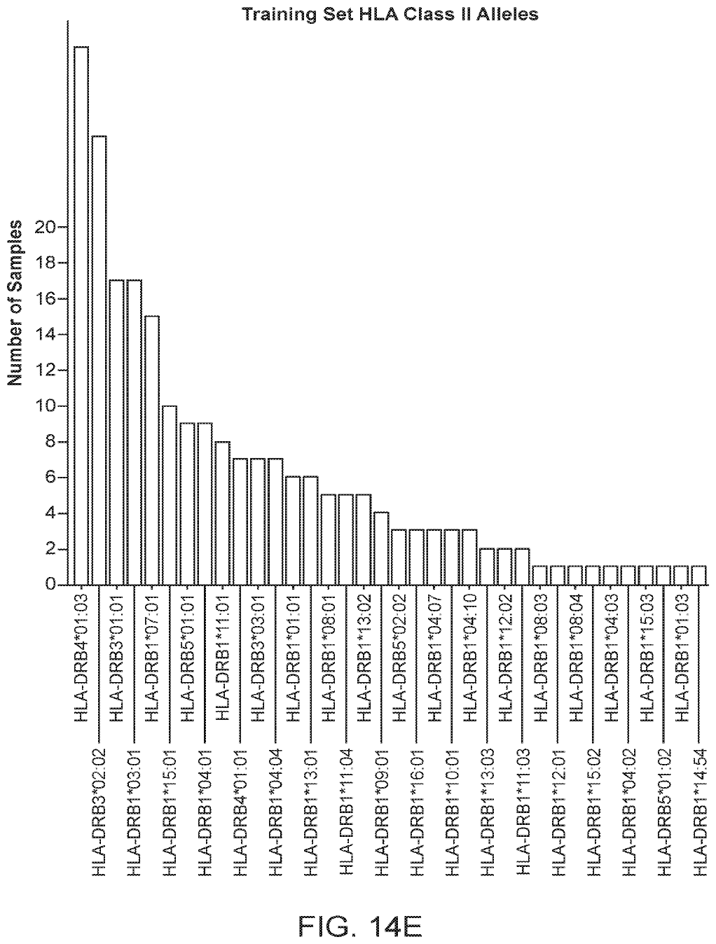

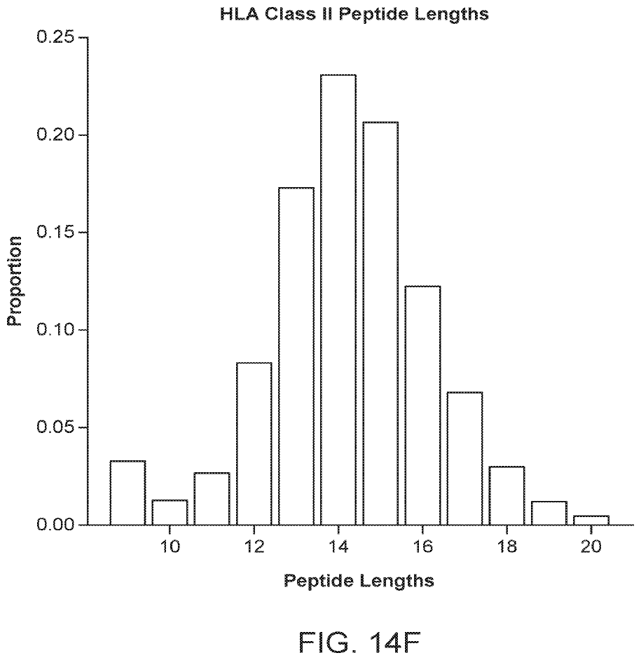

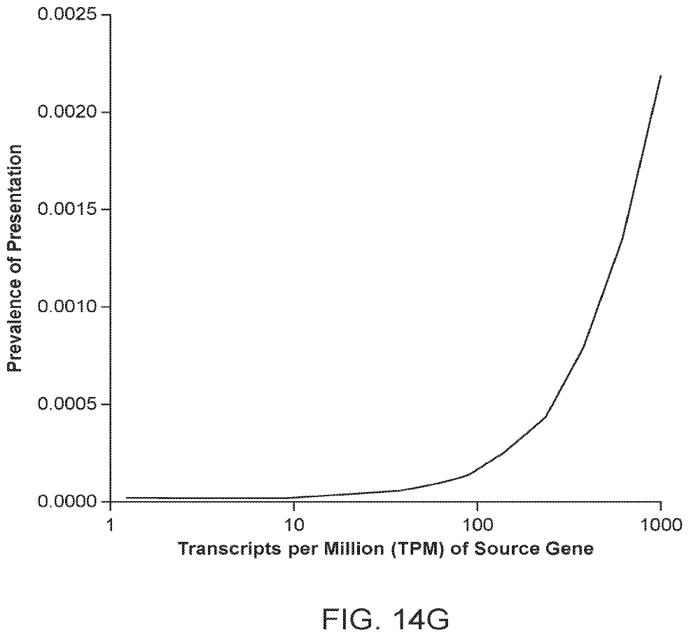

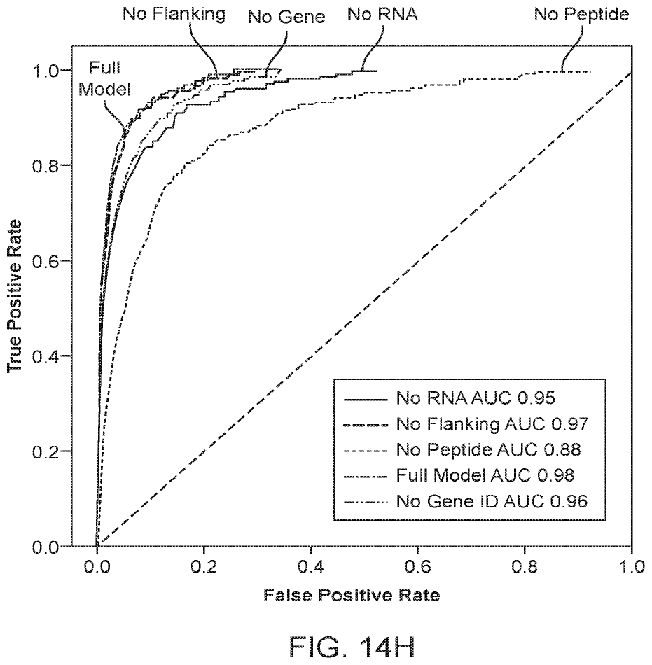

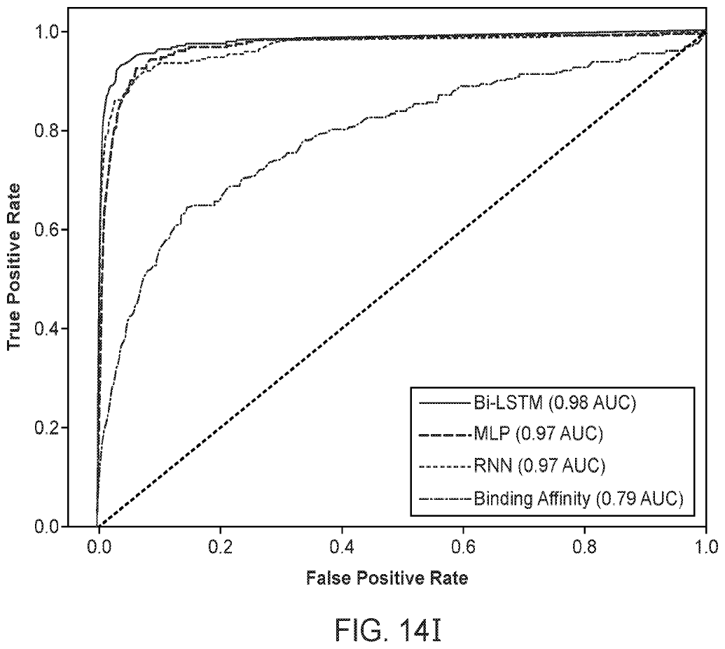

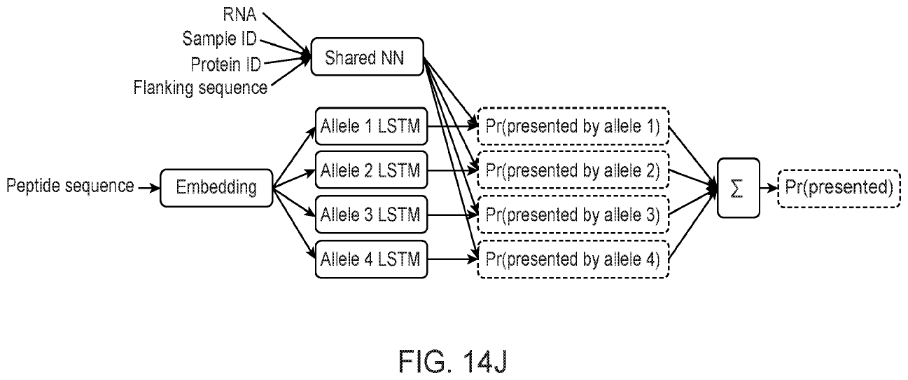

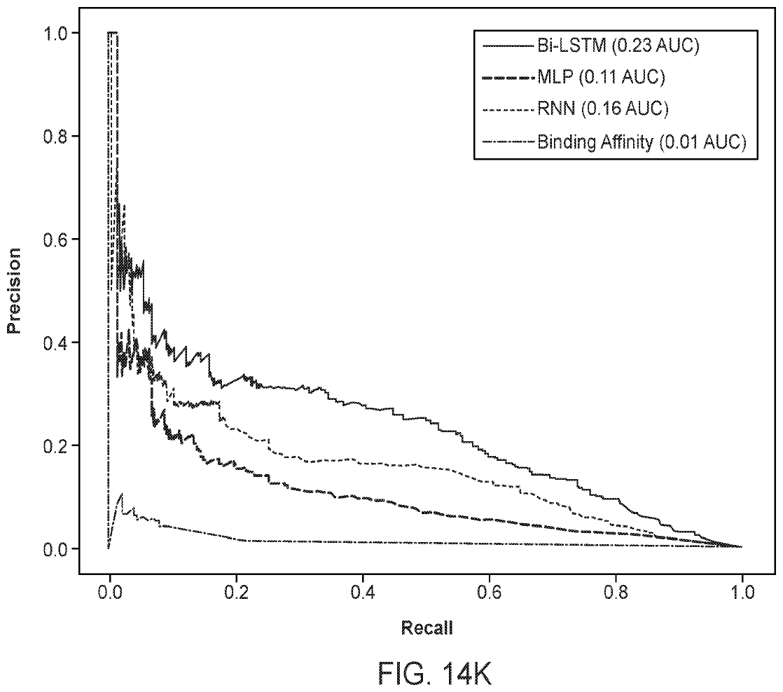

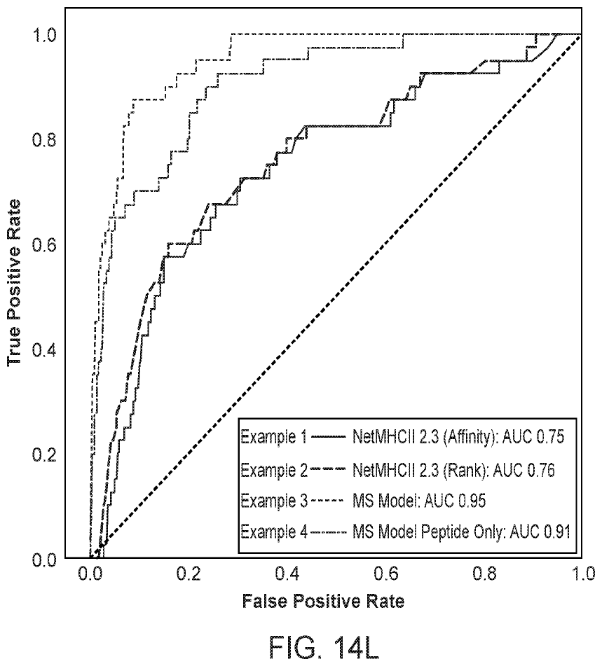

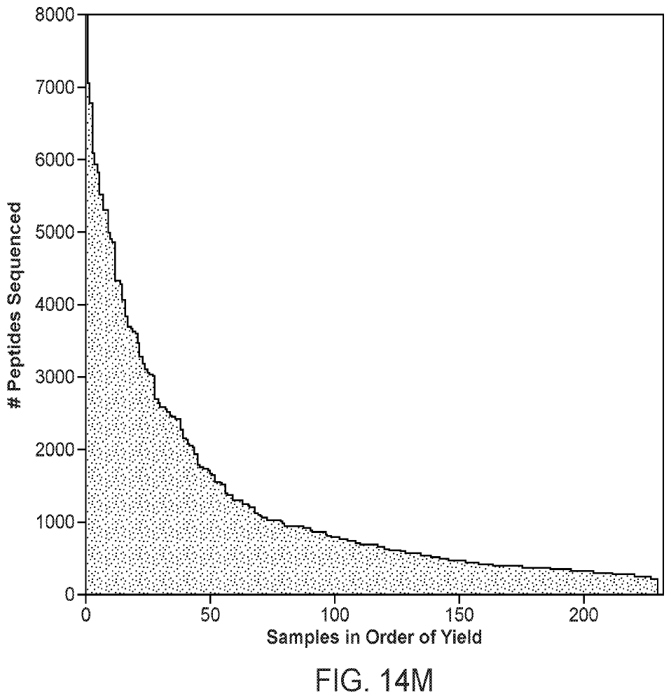

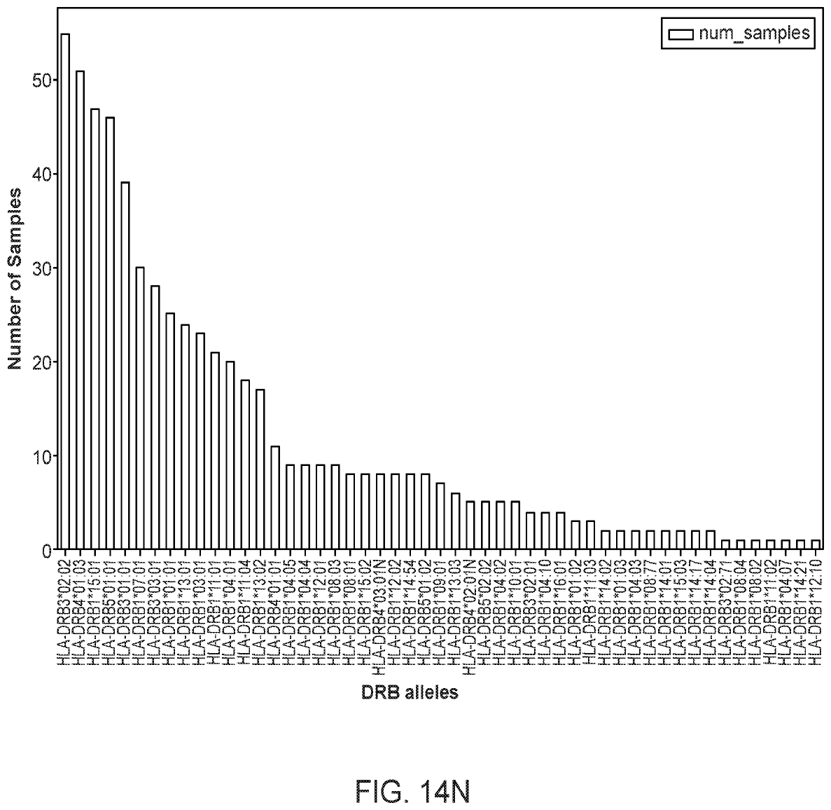

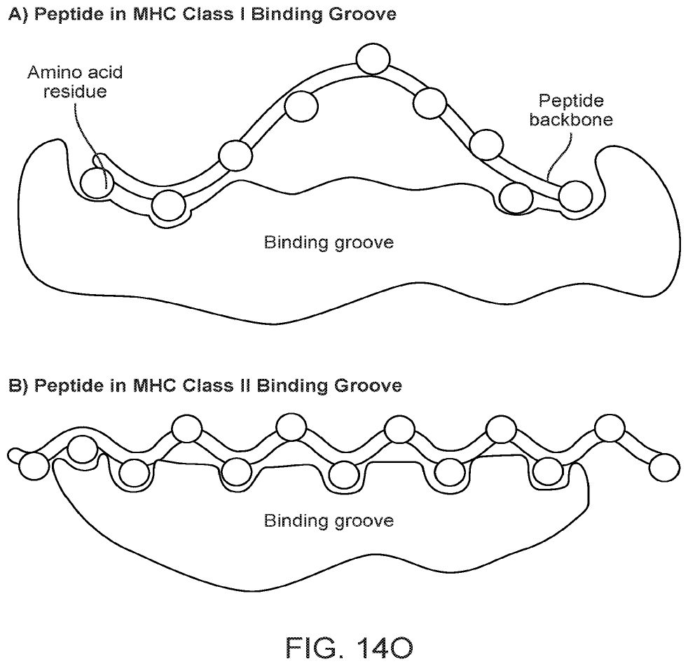

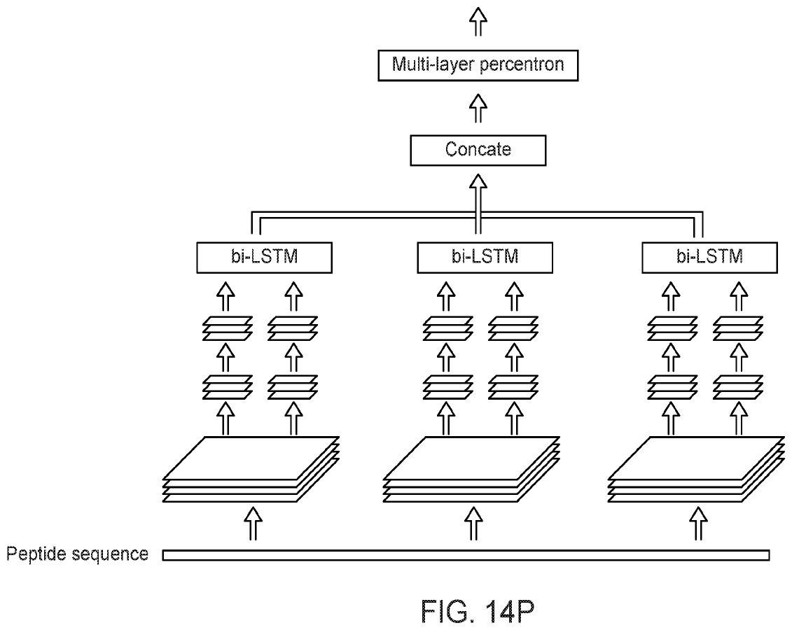

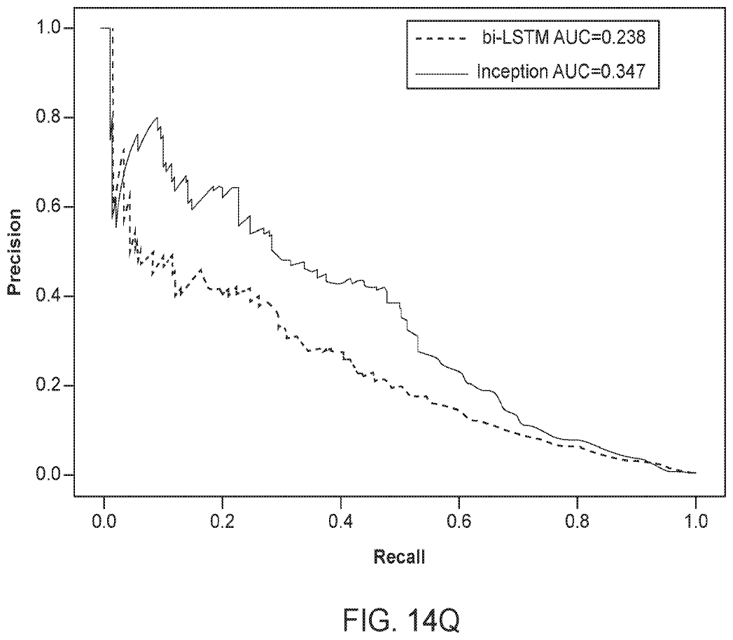

Allele-noninteracting information can also include mRNA quantification measurements. For example, mRNA quantification data can be obtained for the same samples that provide the mass spectrometry training data. As later described in reference to FIG. 14G, RNA expression was identified to be a strong predictor of peptide presentation. In one embodiment, the mRNA quantification measurements are identified from software tool RSEM. Detailed implementation of the RSEM software tool can be found at Bo Li and Colin N. Dewey. RSEM: accurate transcript quantification from RNA-Seq data with or without a reference genome. BMC Bioinformatics, 12:323, August 2011. In one embodiment, the mRNA quantification is measured in units of fragments per kilobase of transcript per Million mapped reads (FPKM).

Allele-noninteracting information can also include the N-terminal sequences flanking the peptide within its source protein sequence.

Allele-noninteracting information can also include the source gene of the peptide sequence. The source gene may be defined as the Ensembl protein family of the peptide sequence. In other examples, the source gene may be defined as the source DNA or the source RNA of the peptide sequence. The source gene can, for example, be represented as a string of nucleotides that encode for a protein, or alternatively be more categorically represented based on a named set of known DNA or RNA sequences that are known to encode specific proteins. In another example, allele-noninteracting information can also include the source transcript or isoform or set of potential source transcripts or isoforms of the peptide sequence drawn from a database such as Ensembl or RefSeq.

Allele-noninteracting information can also include the tissue type, cell type or tumor type of cells of origin of the peptide sequence.

Allele-noninteracting information can also include the presence of protease cleavage motifs in the peptide, optionally weighted according to the expression of corresponding proteases in the tumor cells (as measured by RNA-seq or mass spectrometry). Peptides that contain protease cleavage motifs are less likely to be presented, because they will be more readily degraded by proteases, and will therefore be less stable within the cell.

Allele-noninteracting information can also include the turnover rate of the source protein as measured in the appropriate cell type. Faster turnover rate (i.e., lower half-life) increases the probability of presentation; however, the predictive power of this feature is low if measured in a dissimilar cell type.

Allele-noninteracting information can also include the length of the source protein, optionally considering the specific splice variants (“isoforms”) most highly expressed in the tumor cells as measured by RNA-seq or proteome mass spectrometry, or as predicted from the annotation of germline or somatic splicing mutations detected in DNA or RNA sequence data.

Allele-noninteracting information can also include the level of expression of the proteasome, immunoproteasome, thymoproteasome, or other proteases in the tumor cells (which may be measured by RNA-seq, proteome mass spectrometry, or immunohistochemistry). Different proteasomes have different cleavage site preferences. More weight will be given to the cleavage preferences of each type of proteasome in proportion to its expression level.

Allele-noninteracting information can also include the expression of the source gene of the peptide (e.g., as measured by RNA-seq or mass spectrometry). Possible optimizations include adjusting the measured expression to account for the presence of stromal cells and tumor-infiltrating lymphocytes within the tumor sample. Peptides from more highly expressed genes are more likely to be presented. Peptides from genes with undetectable levels of expression can be excluded from consideration.

Allele-noninteracting information can also include the probability that the source mRNA of the neoantigen encoded peptide will be subject to nonsense-mediated decay as predicted by a model of nonsense-mediated decay, for example, the model from Rivas et al. Science 2015.

Allele-noninteracting information can also include the typical tissue-specific expression of the source gene of the peptide during various stages of the cell cycle. Genes that are expressed at a low level overall (as measured by RNA-seq or mass spectrometry proteomics) but that are known to be expressed at a high level during specific stages of the cell cycle are likely to produce more presented peptides than genes that are stably expressed at very low levels.

Allele-noninteracting information can also include a comprehensive catalog of features of the source protein as given in e.g. uniProt or PDB http://www.rcsb.org/pdb/home/home.do. These features may include, among others: the secondary and tertiary structures of the protein, subcellular localization 11, Gene ontology (GO) terms. Specifically, this information may contain annotations that act at the level of the protein, e.g., 5′ UTR length, and annotations that act at the level of specific residues, e.g., helix motif between residues 300 and 310. These features can also include turn motifs, sheet motifs, and disordered residues.

Allele-noninteracting information can also include features describing the properties of the domain of the source protein containing the peptide, for example: secondary or tertiary structure (e.g., alpha helix vs beta sheet): Alternative splicing.

Allele-noninteracting information can also include features describing the presence or absence of a presentation hotspot at the position of the peptide in the source protein of the peptide.

Allele-noninteracting information can also include the probability of presentation of peptides from the source protein of the peptide in question in other individuals (after adjusting for the expression level of the source protein in those individuals and the influence of the different HLA types of those individuals).

Allele-noninteracting information can also include the probability that the peptide will not be detected or over-represented by mass spectrometry due to technical biases.

The expression of various gene modules/pathways as measured by a gene expression assay such as RNASeq, microarray(s), targeted panel(s) such as Nanostring, or single/multi-gene representatives of gene modules measured by assays such as RT-PCR (which need not contain the source protein of the peptide) that are informative about the state of the tumor cells, stroma, or tumor-infiltrating lymphocytes (TILs).

Allele-noninteracting information can also include the copy number of the source gene of the peptide in the tumor cells. For example, peptides from genes that are subject to homozygous deletion in tumor cells can be assigned a probability of presentation of zero.

Allele-noninteracting information can also include the probability that the peptide binds to the TAP or the measured or predicted binding affinity of the peptide to the TAP. Peptides that are more likely to bind to the TAP, or peptides that bind the TAP with higher affinity are more likely to be presented by MHC-I.

Allele-noninteracting information can also include the expression level of TAP in the tumor cells (which may be measured by RNA-seq, proteome mass spectrometry, immunohistochemistry). For MHC-I, higher TAP expression levels increase the probability of presentation of all peptides.

Allele-noninteracting information can also include the presence or absence of tumor mutations, including, but not limited to:
i. Driver mutations in known cancer driver genes such as EGFR, KRAS, ALK, RET, ROS1, TP53, CDKN2A, CDKN2B, NTRK1, NTRK2, NTRK3
ii. In genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOBHLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome). Peptides whose presentation relies on a component of the antigen-presentation machinery that is subject to loss-of-function mutation in the tumor have reduced probability of presentation.

Presence or absence of functional germline polymorphisms, including, but not limited to:
i. In genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOBHLA-DP, HLA-DPA 1, HLA-DPB1, HLA-DQ, HLA-DQA1, HLA-DQA2. HLA-DQB1, HLA-DQB2, HLA-DR, HLA-DRA. HLA-DRB1, HLA-DRB3, HLA-DRB4, HLA-DRB5 or any of the genes coding for components of the proteasome or immunoproteasome)

Allele-noninteracting information can also include tumor type (e.g., NSCLC, melanoma).

Allele-noninteracting information can also include known functionality of HLA alleles, as reflected by, for instance HLA allele suffixes. For example, the N suffix in the allele name HLA-A*24:09N indicates a null allele that is not expressed and is therefore unlikely to present epitopes; the full HLA allele suffix nomenclature is described at https://www.ebi.ac.uk/ipd/imgt/hla/nomenclature/suffixes.html.

Allele-noninteracting information can also include clinical tumor subtype (e.g., squamous lung cancer vs. non-squamous).

Allele-noninteracting information can also include smoking history.

Allele-noninteracting information can also include history of sunburn, sun exposure, or exposure to other mutagens.

Allele-noninteracting information can also include the typical expression of the source gene of the peptide in the relevant tumor type or clinical subtype, optionally stratified by driver mutation. Genes that are typically expressed at high levels in the relevant tumor type are more likely to be presented.

Allele-noninteracting information can also include the frequency of the mutation in all tumors, or in tumors of the same type, or in tumors from individuals with at least one shared MHC allele, or in tumors of the same type in individuals with at least one shared MHC allele.

In the case of a mutated tumor-specific peptide, the list of features used to predict a probability of presentation may also include the annotation of the mutation (e.g., missense, read-through, frameshift, fusion, etc.) or whether the mutation is predicted to result in nonsense-mediated decay (NMD). For example, peptides from protein segments that are not translated in tumor cells due to homozygous early-stop mutations can be assigned a probability of presentation of zero. NMD results in decreased mRNA translation, which decreases the probability of presentation.

### VII.C. Presentation Identification System

FIG. 3 is a high-level block diagram illustrating the computer logic components of the presentation identification system 160, according to one embodiment. In this example embodiment, the presentation identification system 160 includes a data management module 312, an encoding module 314, a training module 316, and a prediction module 320. The presentation identification system 160 is also comprised of a training data store 170 and a presentation models store 175. Some embodiments of the model management system 160 have different modules than those described here. Similarly, the functions can be distributed among the modules in a different manner than is described here.

### VII.C.1. Data Management Module

The data management module 312 generates sets of training data 170 from the presentation information 165. Each set of training data contains a plurality of data instances, in which each data instance i contains a set of independent variables zⁱ that include at least a presented or non-presented peptide sequence pⁱ, one or more associated MHC alleles aⁱ associated with the peptide sequence pⁱ, and a dependent variable yⁱ that represents information that the presentation identification system 160 is interested in predicting for new values of independent variables.

In one particular implementation referred throughout the remainder of the specification, the dependent variable yⁱ is a binary label indicating whether peptide pⁱ was presented by the one or more associated MHC alleles aⁱ. However, it is appreciated that in other implementations, the dependent variable yⁱ can represent any other kind of information that the presentation identification system 160 is interested in predicting dependent on the independent variables zⁱ. For example, in another implementation, the dependent variable yⁱ may also be a numerical value indicating the mass spectrometry ion current identified for the data instance.

The peptide sequence pⁱ for data instance i is a sequence of kᵢ amino acids, in which kᵢ may vary between data instances i within a range. For example, that range may be 8-15 for MHC class I or 6-30 for MHC class II. In one specific implementation of system 160, all peptide sequences pⁱ in a training data set may have the same length, e.g. 9. The number of amino acids in a peptide sequence may vary depending on the type of MHC alleles (e.g., MHC alleles in humans, etc.). The MHC alleles aⁱ for data instance i indicate which MHC alleles were present in association with the corresponding peptide sequence pⁱ.

The data management module 312 may also include additional allele-interacting variables, such as binding affinity bⁱ and stability s predictions in conjunction with the peptide sequences pⁱ and associated MHC alleles aⁱ contained in the training data 170. For example, the training data 170 may contain binding affinity predictions bⁱ between a peptide pⁱ and each of the associated MHC molecules indicated in aⁱ. As another example, the training data 170 may contain stability predictions s for each of the MHC alleles indicated in aⁱ.

The data management module 312 may also include allele-noninteracting variables wⁱ, such as C-terminal flanking sequences and mRNA quantification measurements in conjunction with the peptide sequences pⁱ.

The data management module 312 also identifies peptide sequences that are not presented by MHC alleles to generate the training data 170. Generally, this involves identifying the “longer” sequences of source protein that include presented peptide sequences prior to presentation. When the presentation information contains engineered cell lines, the data management module 312 identifies a series of peptide sequences in the synthetic protein to which the cells were exposed to that were not presented on MHC alleles of the cells. When the presentation information contains tissue samples, the data management module 312 identifies source proteins from which presented peptide sequences originated from, and identifies a series of peptide sequences in the source protein that were not presented on MHC alleles of the tissue sample cells.

The data management module 312 may also artificially generate peptides with random sequences of amino acids and identify the generated sequences as peptides not presented on MHC alleles. This can be accomplished by randomly generating peptide sequences allows the data management module 312 to easily generate large amounts of synthetic data for peptides not presented on MHC alleles. Since in reality, a small percentage of peptide sequences are presented by MHC alleles, the synthetically generated peptide sequences are highly likely not to have been presented by MHC alleles even if they were included in proteins processed by cells.

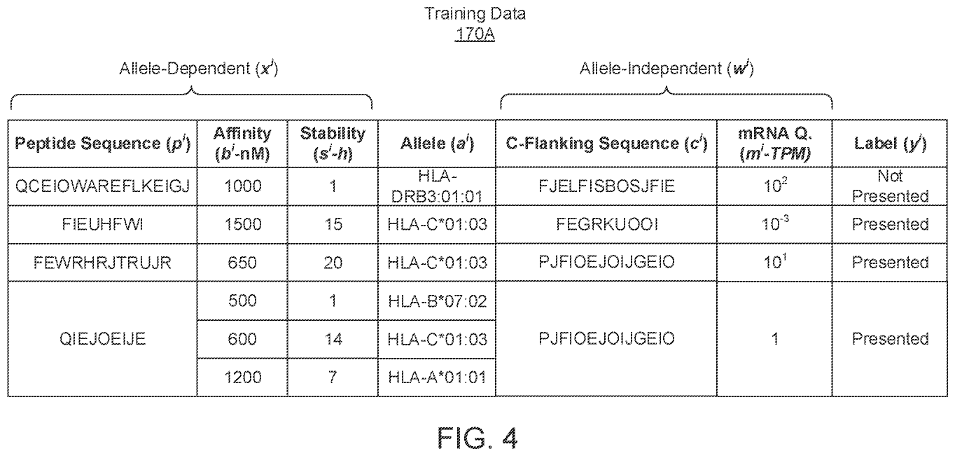

FIG. 4 illustrates an example set of training data 170A, according to one embodiment. Specifically, the first 3 data instances in the training data 170A indicate peptide presentation information from a single-allele cell line involving the allele HLA-C*01:03 and 3 peptide sequences QCEIOWAREFLKEIGJ, FIEUHFWI, and FEWRHRJTRUJR. The fourth data instance in the training data 170A indicates peptide information from a multiple-allele cell line involving the alleles HLA-B*07:02, HLA-C*01:03, HLA-A*01:01 and a peptide sequence QIEJOEIJE. The first data instance indicates that peptide sequence QCEIOWARE was not presented by the allele HLA-DRB3:01:01. As discussed in the prior two paragraphs, the negatively-labeled peptide sequences may be randomly generated by the data management module 312 or identified from source protein of presented peptides. The training data 170A also includes a binding affinity prediction of 1000 nM and a stability prediction of a half-life of 1 h for the peptide sequence-allele pair. The training data 170A also includes allele-noninteracting variables, such as the C-terminal flanking sequence of the peptide FJELFISBOSJFIE, and a mRNA quantification measurement of 10² TPM. The fourth data instance indicates that peptide sequence QIEJOEIJE was presented by one of the alleles HLA-B*07:02, HLA-C*01:03, or HLA-A*01:01. The training data 170A also includes binding affinity predictions and stability predictions for each of the alleles, as well as the C-terminal flanking sequence of the peptide and the mRNA quantification measurement for the peptide.

### VII.C.2. Encoding Module

The encoding module 314 encodes information contained in the training data 170 into a numerical representation that can be used to generate the one or more presentation models. In one implementation, the encoding module 314 one-hot encodes sequences (e.g., peptide sequences or C-terminal flanking sequences) over a predetermined 20-letter amino acid alphabet. Specifically, a peptide sequence pⁱ with kᵢ amino acids is represented as a row vector of 20·kᵢ elements, where a single element among pⁱ_{20·(j-1)+1}, pⁱ_{20·(j-1)+2}, . . . , pⁱ_{20·j} that corresponds to the alphabet of the amino acid at the j-th position of the peptide sequence has a value of 1. Otherwise, the remaining elements have a value of 0. As an example, for a given alphabet {A, C, D, E, F, G, H, I, K, L, M, N, P, Q, R, S, T, V, W, Y}, the peptide sequence EAF of 3 amino acids for data instance i may be represented by the row vector of 60 elements pⁱ=[0 0 0 1 0 0 0 0 0 0 0 0 0 0 0 0 0 0 0 0 1 0 0 0 0 0 0 0 0 0 0 0 0 0 0 0 0 0 0 0 0 0 0 0 1 0 0 0 0 0 0 0 0 0 0 0 0 0 0 0]. The C-terminal flanking sequence cⁱ can be similarly encoded as described above, as well as the protein sequence dₕ for MHC alleles, and other sequence data in the presentation information.

When the training data 170 contains sequences of differing lengths of amino acids, the encoding module 314 may further encode the peptides into equal-length vectors by adding a PAD character to extend the predetermined alphabet. For example, this may be performed by left-padding the peptide sequences with the PAD character until the length of the peptide sequence reaches the peptide sequence with the greatest length in the training data 170. Thus, when the peptide sequence with the greatest length has kₘₐₓ amino acids, the encoding module 314 numerically represents each sequence as a row vector of (20+l)·kₘₐₓ elements. As an example, for the extended alphabet {PAD, A, C, D, E, F, G, H, I, K, L, M, N, P, Q, R, S, T, V, W, Y} and a maximum amino acid length of kₘₐₓ=5, the same example peptide sequence EAF of 3 amino acids may be represented by the row vector of 105 elements pⁱ=[1 0 0 0 0 0 0 0 0 0 0 0 0 0 0 0 0 0 0 0 0 1 0 0 0 0 0 0 0 0 0 0 0 0 0 0 0 0 0 0 0 0 0 0 0 0 1 0 0 0 0 0 0 0 0 0 0 0 0 0 0 0 0 0 1 0 0 0 0 0 0 0 0 0 0 0 0 0 0 0 0 0 0 0 0 0 0 0 0 0 0 0 0 0 0 0 0 0 0 0 0 0 0 0]. The C-terminal flanking sequence cⁱ or other sequence data can be similarly encoded as described above. Thus, each independent variable or column in the peptide sequence pⁱ or cⁱ represents presence of a particular amino acid at a particular position of the sequence.

Although the above method of encoding sequence data was described in reference to sequences having amino acid sequences, the method can similarly be extended to other types of sequence data, such as DNA or RNA sequence data, and the like.

The encoding module 314 also encodes the one or more MHC alleles aⁱ for data instance i as a row vector of m elements, in which each element h=1, 2, . . . , m corresponds to a unique identified MHC allele. The elements corresponding to the MHC alleles identified for the data instance i have a value of 1. Otherwise, the remaining elements have a value of 0. As an example, the alleles HLA-B*07:02 and HLA-DRB1*10:01 for a data instance i corresponding to a multiple-allele cell line among m=4 unique identified MHC allele types {HLA-A*01:01, HLA-C*01:08, HLA-B*07:02, HLA-DRB1*10:01} may be represented by the row vector of 4 elements aⁱ=[0 0 1 1], in which a₃ⁱ=1 and a₄ⁱ=1. Although the example is described herein with 4 identified MHC allele types, the number of MHC allele types can be hundreds or thousands in practice. As previously discussed, each data instance i typically contains at most 6 different MHC class I allele types in association with the peptide sequence pᵢ and/or at most 4 different MHC class II DR allele types in association with the peptide sequence pᵢ, and/or at most 12 different MHC class II allele types in association with the peptide sequence pᵢ.

The encoding module 314 also encodes the label yᵢ for each data instance i as a binary variable having values from the set of {0, 1}, in which a value of 1 indicates that peptide xⁱ was presented by one of the associated MHC alleles aⁱ, and a value of 0 indicates that peptide xⁱ was not presented by any of the associated MHC alleles aⁱ. When the dependent variable yᵢ represents the mass spectrometry ion current, the encoding module 314 may additionally scale the values using various functions, such as the log function having a range of (−∞, ∞) for ion current values between [0, ∞).

The encoding module 314 may represent a pair of allele-interacting variables xₕⁱ for peptide pᵢ and an associated MHC allele h as a row vector in which numerical representations of allele-interacting variables are concatenated one after the other. For example, the encoding module 314 may represent xₕⁱ as a row vector equal to [pⁱ], [pⁱ bₕⁱ], [pⁱ sₕⁱ], or [pⁱ bₕⁱ sₕⁱ], where bₕⁱ is the binding affinity prediction for peptide pᵢ and associated MHC allele h, and similarly for sₕⁱ for stability. Alternatively, one or more combination of allele-interacting variables may be stored individually (e.g., as individual vectors or matrices).

In one instance, the encoding module 314 represents binding affinity information by incorporating measured or predicted values for binding affinity in the allele-interacting variables xₕⁱ.

In one instance, the encoding module 314 represents binding stability information by incorporating measured or predicted values for binding stability in the allele-interacting variables xₕⁱ.

In one instance, the encoding module 314 represents binding on-rate information by incorporating measured or predicted values for binding on-rate in the allele-interacting variables xₕⁱ.

In one instance, for peptides presented by class I MHC molecules, the encoding module 314 represents peptide length as a vector Tₖ=[ [symbol] (Lₖ=8) [symbol] (Lₖ=9) [symbol] (Lₖ=10) [symbol] (Lₖ=11) [symbol] (Lₖ=12) [symbol] (Lₖ=13) [symbol] (Lₖ=14) [symbol] (Lₖ=15)] where [symbol] is the indicator function, and Lₖ denotes the length of peptide pₖ. The vector Tₖ can be included in the allele-interacting variables xₕⁱ. In another instance, for peptides presented by class II MHC molecules, the encoding module 314 represents peptide length as a vector Tₖ=[ [symbol] (Lₖ=6) [symbol] (Lₖ=7) [symbol] (Lₖ=8) [symbol] (Lₖ=9) [symbol] (Lₖ=10) [symbol] (Lₖ=11) [symbol] (Lₖ=12) [symbol] (Lₖ=13) [symbol] (Lₖ=14) [symbol] (Lₖ=15) [symbol] (Lₖ=16) [symbol] (Lₖ=17) [symbol] (Lₖ=18) [symbol] (Lₖ=19) [symbol] (Lₖ=20) [symbol] (Lₖ=21) [symbol] (Lₖ=22) [symbol] (Lₖ=23) [symbol] (Lₖ=24) [symbol] (Lₖ=25) [symbol] (Lₖ=26) [symbol] (Lₖ=27) [symbol] (Lₖ=28) [symbol] (Lₖ=29) [symbol] (Lₖ=30)] where [symbol] is the indicator function, and Lₖ denotes the length of peptide pₖ. The vector Tₖ can be included in the allele-interacting variables xₕⁱ.

In one instance, the encoding module 314 represents RNA expression information of MHC alleles by incorporating RNA-seq based expression levels of MHC alleles in the allele-interacting variables xₕⁱ.

Similarly, the encoding module 314 may represent the allele-noninteracting variables wⁱ as a row vector in which numerical representations of allele-noninteracting variables are concatenated one after the other. For example, wⁱ may be a row vector equal to [cⁱ] or [cⁱ mⁱ wⁱ] in which wⁱ is a row vector representing any other allele-noninteracting variables in addition to the C-terminal flanking sequence of peptide pⁱ and the mRNA quantification measurement W associated with the peptide. Alternatively, one or more combination of allele-noninteracting variables may be stored individually (e.g., as individual vectors or matrices).

In one instance, the encoding module 314 represents turnover rate of source protein for a peptide sequence by incorporating the turnover rate or half-life in the allele-noninteracting variables wⁱ.

In one instance, the encoding module 314 represents length of source protein or isoform by incorporating the protein length in the allele-noninteracting variables wⁱ.

In one instance, the encoding module 314 represents activation of immunoproteasome by incorporating the mean expression of the immunoproteasome-specific proteasome subunits including the β1ᵢ, β2ᵢ, β5ᵢ subunits in the allele-noninteracting variables wⁱ.

In one instance, the encoding module 314 represents the RNA-seq abundance of the source protein of the peptide or gene or transcript of a peptide (quantified in units of FPKM, TPM by techniques such as RSEM) can be incorporating the abundance of the source protein in the allele-noninteracting variables wⁱ.

In one instance, the encoding module 314 represents the probability that the transcript of origin of a peptide will undergo nonsense-mediated decay (NMD) as estimated by the model in, for example, Rivas et. al. Science, 2015 by incorporating this probability in the allele-noninteracting variables wⁱ.

In one instance, the encoding module 314 represents the activation status of a gene module or pathway assessed via RNA-seq by, for example, quantifying expression of the genes in the pathway in units of TPM using e.g., RSEM for each of the genes in the pathway then computing a summary statistics, e.g., the mean, across genes in the pathway. The mean can be incorporated in the allele-noninteracting variables wⁱ.

In one instance, the encoding module 314 represents the copy number of the source gene by incorporating the copy number in the allele-noninteracting variables wⁱ.

In one instance, the encoding module 314 represents the TAP binding affinity by including the measured or predicted TAP binding affinity (e.g., in nanomolar units) in the allele-noninteracting variables wⁱ.

In one instance, the encoding module 314 represents TAP expression levels by including TAP expression levels measured by RNA-seq (and quantified in units of TPM by e.g., RSEM) in the allele-noninteracting variables wⁱ.

In one instance, the encoding module 314 represents tumor mutations as a vector of indicator variables (i.e., d^{k}=1 if peptide p^{k} comes from a sample with a KRAS G12D mutation and 0 otherwise) in the allele-noninteracting variables wⁱ.

In one instance, the encoding module 314 represents germline polymorphisms in antigen presentation genes as a vector of indicator variables (i.e., d^{k}=1 if peptide p^{k} comes from a sample with a specific germline polymorphism in the TAP). These indicator variables can be included in the allele-noninteracting variables wⁱ.

In one instance, the encoding module 314 represents tumor type as a length-one one-hot encoded vector over the alphabet of tumor types (e.g., NSCLC, melanoma, colorectal cancer, etc). These one-hot-encoded variables can be included in the allele-noninteracting variables wⁱ.

In one instance, the encoding module 314 represents MHC allele suffixes by treating 4-digit HLA alleles with different suffixes. For example, HLA-A*24:09N is considered a different allele from HLA-A*24:09 for the purpose of the model. Alternatively, the probability of presentation by an N-suffixed MHC allele can be set to zero for all peptides, because HLA alleles ending in the N suffix are not expressed.

In one instance, the encoding module 314 represents tumor subtype as a length-one one-hot encoded vector over the alphabet of tumor subtypes (e.g., lung adenocarcinoma, lung squamous cell carcinoma, etc). These one-hot encoded variables can be included in the allele-noninteracting variables wⁱ.

In one instance, the encoding module 314 represents smoking history as a binary indicator variable (d^{k}=1 if the patient has a smoking history, and 0 otherwise), that can be included in the allele-noninteracting variables wⁱ. Alternatively, smoking history can be encoded as a length-one one-hot encoded variable over an alphabet of smoking severity. For example, smoking status can be rated on a 1-5 scale, where 1 indicates nonsmokers, and 5 indicates current heavy smokers. Because smoking history is primarily relevant to lung tumors, when training a model on multiple tumor types, this variable can also be defined to be equal to 1 if the patient has a history of smoking and the tumor type is lung tumors and zero otherwise.

In one instance, the encoding module 314 represents sunburn history as a binary indicator variable (d^{k}=1 if the patient has a history of severe sunburn, and 0 otherwise), which can be included in the allele-noninteracting variables wⁱ. Because severe sunburn is primarily relevant to melanomas, when training a model on multiple tumor types, this variable can also be defined to be equal to 1 if the patient has a history of severe sunburn and the tumor type is melanoma and zero otherwise.

In one instance, the encoding module 314 represents distribution of expression levels of a particular gene or transcript for each gene or transcript in the human genome as summary statistics (e.g., mean, median) of distribution of expression levels by using reference databases such as TCGA. Specifically, for a peptide p^{k} in a sample with tumor type melanoma, we can include not only the measured gene or transcript expression level of the gene or transcript of origin of peptide p^{k} in the allele-noninteracting variables wⁱ, but also the mean and/or median gene or transcript expression of the gene or transcript of origin of peptide p^{k} in melanomas as measured by TCGA.

In one instance, the encoding module 314 represents mutation type as a length-one one-hot-encoded variable over the alphabet of mutation types (e.g., missense, frameshift, NMD-inducing, etc). These one hot-encoded variables can be included in the allele-noninteracting variables wⁱ.

In one instance, the encoding module 314 represents protein-level features of protein as the value of the annotation (e.g., 5′ UTR length) of the source protein in the allele-noninteracting variables wⁱ. In another instance, the encoding module 314 represents residue-level annotations of the source protein for peptide pⁱ by including an indicator variable, that is equal to 1 if peptide pⁱ overlaps with a helix motif and 0 otherwise, or that is equal to 1 if peptide pⁱ is completely contained with within a helix motif in the allele-noninteracting variables wⁱ. In another instance, a feature representing proportion of residues in peptide pⁱ that are contained within a helix motif annotation can be included in the allele-noninteracting variables wⁱ.

In one instance, the encoding module 314 represents type of proteins or isoforms in the human proteome as an indicator vector o^{k} that has a length equal to the number of proteins or isoforms in the human proteome, and the corresponding element o^{k}ᵢ is I if peptide p^{k} comes from protein i and 0 otherwise.

In one instance, the encoding module 314 represents the source gene G=gene(pⁱ) of peptide pⁱ as a categorical variable with L possible categories, where L denotes the upper limit of the number of indexed source genes 1, 2, . . . , L.

In one instance, the encoding module 314 represents the tissue type, cell type, tumor type, or tumor histology type T=tissue(pⁱ) of peptide pⁱ as a categorical variable with A possible categories, where M denotes the upper limit of the number of indexed types 1, 2, . . . , M. Types of tissue can include, for example, lung tissue, cardiac tissue, intestine tissue, nerve tissue, and the like. Types of cells can include dendritic cells, macrophages, CD4 T cells, and the like. Types of tumors can include lung adenocarcinoma, lung squamous cell carcinoma, melanoma, non-Hodgkin lymphoma, and the like.

The encoding module 314 may also represent the overall set of variables zⁱ for peptide pⁱ and an associated MHC allele h as a row vector in which numerical representations of the allele-interacting variables xⁱ and the allele-noninteracting variables wⁱ are concatenated one after the other. For example, the encoding module 314 may represent zₕⁱ as a row vector equal to [xₕⁱ wⁱ] or [wᵢxₕⁱ].

### VIII. Training Module

The training module 316 constructs one or more presentation models that generate likelihoods of whether peptide sequences will be presented by MHC alleles associated with the peptide sequences. Specifically, given a peptide sequence p^{k} and a set of MHC alleles a^{k} associated with the peptide sequence p^{k}, each presentation model generates an estimate uₖ indicating a likelihood that the peptide sequence p^{k} will be presented by one or more of the associated MHC alleles a^{k}.

### VIII.A. Overview

The training module 316 constructs the one more presentation models based on the training data sets stored in store 170 generated from the presentation information stored in 165. Generally, regardless of the specific type of presentation model, all of the presentation models capture the dependence between independent variables and dependent variables in the training data 170 such that a loss function is minimized. Specifically, the loss function [symbol] (y_{i∈S}, u_{i∈S}; θ) represents discrepancies between values of dependent variables y_{i∈S} for one or more data instances S in the training data 170 and the estimated likelihoods u_{i∈S} for the data instances S generated by the presentation model. In one particular implementation referred throughout the remainder of the specification, the loss function (y_{i∈S}; u_{i∈S}; θ) is the negative log likelihood function given by equation (1a) as follows:

$$ℓ ( {y}_{i ∈ S} , {u}_{i ∈ S} ; θ ) = {\sum }_{i ∈ S}^{ } ( {y}_{i} log {u}_{i} + ( 1 - {y}_{i} ) log ( 1 - {u}_{i} ) ) . \qquad ( 1 a )$$

However, in practice, another loss function may be used. For example, when predictions are made for the mass spectrometry ion current, the loss function is the mean squared loss given by equation 1b as follows:

$$ℓ ( {y}_{i ∈ S} , {u}_{i ∈ S} ; θ ) = {\sum }_{i ∈ S}^{ } ( {‖ {y}_{i} - {u}_{i} ‖}_{2}^{2} ) . \qquad ( 1 b )$$

The presentation model may be a parametric model in which one or more parameters θ mathematically specify the dependence between the independent variables and dependent variables. Typically, various parameters of parametric-type presentation models that minimize the loss function (y_{i∈S}; u_{i∈S}; θ) are determined through gradient-based numerical optimization algorithms, such as batch gradient algorithms, stochastic gradient algorithms, and the like. Alternatively, the presentation model may be a non-parametric model in which the model structure is determined from the training data 170 and is not strictly based on a fixed set of parameters.

### VIII.B. Per-Allele Models

The training module 316 may construct the presentation models to predict presentation likelihoods of peptides on a per-allele basis. In this case, the training module 316 may train the presentation models based on data instances S in the training data 170 generated from cells expressing single MHC alleles.

In one implementation, the training module 316 models the estimated presentation likelihood uₖ for peptide p for a specific allele h by:

uₖ^{h}=Pr(p^{k} presented; MHC allele h)=ƒ(gₕ(xₕ^{k};θₕ)), (2)

where peptide sequence xₕ^{k} denotes the encoded allele-interacting variables for peptide p^{k} and corresponding MHC allele h,ƒ(⋅) is any function, and is herein throughout is referred to as a transformation function for convenience of description. Further, gₕ(⋅) is any function, is herein throughout referred to as a dependency function for convenience of description, and generates dependency scores for the allele-interacting variables xₕ^{k} based on a set of parameters θₕ determined for MHC allele h. The values for the set of parameters θₕ for each MHC allele h can be determined by minimizing the loss function with respect to θₕ, where i is each instance in the subset S of training data 170 generated from cells expressing the single MHC allele h.

The output of the dependency function gₕ(xₕ^{k};θₕ) represents a dependency score for the MHC allele h indicating whether the MHC allele h will present the corresponding neoantigen based on at least the allele interacting features xₕ^{k}, and in particular, based on positions of amino acids of the peptide sequence of peptide p^{k}. For example, the dependency score for the MHC allele h may have a high value if the MHC allele h is likely to present the peptide p^{k}, and may have a low value if presentation is not likely. The transformation function ƒ(⋅) transforms the input, and more specifically, transforms the dependency score generated by gₕ(xₕ^{k};θₕ) in this case, to an appropriate value to indicate the likelihood that the peptide p^{k} will be presented by an MHC allele.

In one particular implementation referred throughout the remainder of the specification, ƒ(⋅) is a function having the range within [0, 1] for an appropriate domain range. In one example, ƒ(⋅) is the expit function given by:

$$f ( z ) = \frac{exp ( z )}{1 + exp ( z )} . \qquad ( 4 )$$

As another example, ƒ(⋅) can also be the hyperbolic tangent function given by:

ƒ(z)=tan h(z) (5)

when the values for the domain z is equal to or greater than 0. Alternatively, when predictions are made for the mass spectrometry ion current that have values outside the range [0, 1], ƒ(⋅) can be any function such as the identity function, the exponential function, the log function, and the like.

Thus, the per-allele likelihood that a peptide sequence p^{k} will be presented by a MHC allele h can be generated by applying the dependency function gₕ(⋅) for the MHC allele h to the encoded version of the peptide sequence p^{k} to generate the corresponding dependency score. The dependency score may be transformed by the transformation function ƒ(⋅) to generate a per-allele likelihood that the peptide sequence p^{k} will be presented by the MHC allele h.

### VIII.B.1 Dependency Functions for Allele Interacting Variables

In one particular implementation referred throughout the specification, the dependency function gₕ(⋅) is an affine function given by:

gₕ(xₕⁱ;θₕ)=xᵢ^{h}·θₕ. (6)

that linearly combines each allele-interacting variable in xₕ^{k} with a corresponding parameter in the set of parameters θₕ determined for the associated MHC allele h.

In another particular implementation referred throughout the specification, the dependency function gₕ(⋅) is a network function given by:

gₕ(xₕⁱ;θₕ)=NNₕ(xₕⁱ;θh). (7)

represented by a network model NNₕ(⋅) having a series of nodes arranged in one or more layers. A node may be connected to other nodes through connections each having an associated parameter in the set of parameters θₕ. A value at one particular node may be represented as a sum of the values of nodes connected to the particular node weighted by the associated parameter mapped by an activation function associated with the particular node. In contrast to the affine function, network models are advantageous because the presentation model can incorporate non-linearity and process data having different lengths of amino acid sequences. Specifically, through non-linear modeling, network models can capture interaction between amino acids at different positions in a peptide sequence and how this interaction affects peptide presentation.

In general, network models NNₕ(⋅) may be structured as feed-forward networks, such as artificial neural networks (ANN), convolutional neural networks (CNN), deep neural networks (DNN), and/or recurrent neural networks (RNN), such as long short-term memory networks (LSTM), bi-directional LSTM networks, bi-directional recurrent networks, deep bi-directional recurrent networks, multi-layer perceptron networks (MLP), and the like.

In one instance referred throughout the remainder of the specification, each MHC allele in h=1, 2, . . . , m is associated with a separate network model, and NNₕ(⋅) denotes the output(s) from a network model associated with MHC allele h.

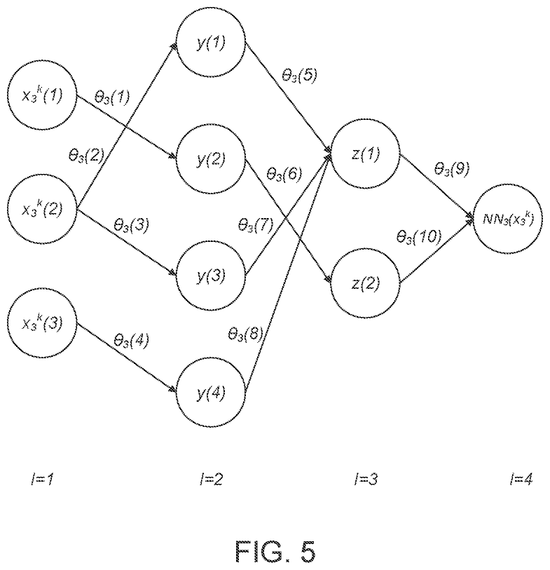

FIG. 5 illustrates an example network model NN₃(⋅) in association with an arbitrary MHC allele h=3. As shown in FIG. 5, the network model NN₃(⋅) for MHC allele h=3 includes three input nodes at layer l=1, four nodes at layer l=2, two nodes at layer l=3, and one output node at layer l=4. The network model NN₃(⋅) is associated with a set of ten parameters θ₃(1), θ₃(2), . . . , θ₃(10). The network model NN₃(⋅) receives input values (individual data instances including encoded polypeptide sequence data and any other training data used) for three allele-interacting variables x₃^{k}(1), x₃^{k}(2), and x₃^{k}(3) for MHC allele h=3 and outputs the value NN₃(x₃^{k}). The network function may also include one or more network models each taking different allele interacting variables as input.

In another instance, the identified MHC alleles h=1, 2, . . . , m are associated with a single network model NN_{H}(⋅), and NNₕ(⋅) denotes one or more outputs of the single network model associated with MHC allele h. In such an instance, the set of parameters A may correspond to a set of parameters for the single network model, and thus, the set of parameters θₕ may be shared by all MHC alleles.

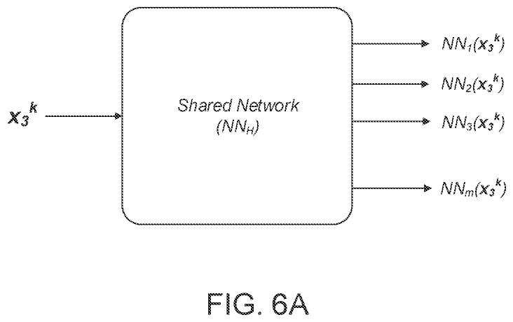

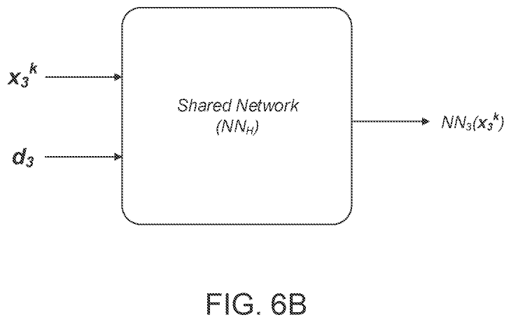

FIG. 6A illustrates an example network model NN_{H}(⋅) shared by MHC alleles h=1, 2, . . . , m. As shown in FIG. 6A, the network model NN_{H}(⋅) includes m output nodes each corresponding to an MHC allele. The network model NN₃(⋅) receives the allele-interacting variables x₃^{k} for MHC allele h=3 and outputs m values including the value NN₃(x₃^{k}) corresponding to the MHC allele h=3.

In yet another instance, the single network model NN_{H}(⋅) may be a network model that outputs a dependency score given the allele interacting variables xₕ^{k} and the encoded protein sequence dₕ of an MHC allele h. In such an instance, the set of parameters θₕ may again correspond to a set of parameters for the single network model, and thus, the set of parameters θₕ may be shared by all MHC alleles. Thus, in such an instance, NNₕ(⋅) may denote the output of the single network model NN_{H}(⋅) given inputs [xₕ^{k} dₕ] to the single network model. Such a network model is advantageous because peptide presentation probabilities for MHC alleles that were unknown in the training data can be predicted just by identification of their protein sequence.

FIG. 6B illustrates an example network model NN_{H}(⋅) shared by MHC alleles. As shown in FIG. 6B, the network model NN_{H}(⋅) receives the allele interacting variables and protein sequence of MHC allele h=3 as input, and outputs a dependency score NN₃(x₃^{k}) corresponding to the MHC allele h=3.

In yet another instance, the dependency function gₕ(⋅) can be expressed as:

gₕ(xₕ^{k};θₕ)=g′ₕ(xₕ^{k};θ′ₕ)+θₕ⁰

where g′ₕ(xₕ^{k};θ′ₕ) is the affine function with a set of parameters θ′ₕ, the network function, or the like, with a bias parameter θₕ⁰ in the set of parameters for allele interacting variables for the MHC allele that represents a baseline probability of presentation for the MHC allele h.

In another implementation, the bias parameter θₕ⁰ may be shared according to the gene family of the MHC allele h. That is, the bias parameter θₕ⁰ for MHC allele h may be equal to θ_{gene(h)}⁰, where gene(h) is the gene family of MHC allele h. For example, class I MHC alleles HLA-A*02:01, HLA-A*02:02, and HLA-A*02:03 may be assigned to the gene family of “HLA-A,” and the bias parameter θₕ⁰ for each of these MHC alleles may be shared. As another example, class II MHC alleles HLA-DRB1:10:01, HLA-DRB1:11:01, and HLA-DRB3:01:01 may be assigned to the gene family of “HLA-DRB,” and the bias parameter θₕ⁰ for each of these MHC alleles may be shared.

Returning to equation (2), as an example, the likelihood that peptide p^{k} will be presented by MHC allele h=3, among m=4 different identified MHC alleles using the affine dependency function gₕ(⋅), can be generated by:

uₖ³=ƒ(x₃^{k}˜θ₃),

where x₃^{k} are the identified allele-interacting variables for MHC allele h=3, and θ₃ are the set of parameters determined for MHC allele h=3 through loss function minimization.

As another example, the likelihood that peptide p^{k} will be presented by MHC allele h=3, among m=4 different identified MHC alleles using separate network transformation functions gₕ(⋅), can be generated by:

uₖ³=ƒ(NN₃(xₖ³;θ₃)),

where xₖ³ are the identified allele-interacting variables for MHC allele h=3, and θ₃ are the set of parameters determined for the network model NN₃(⋅) associated with MHC allele h=3.

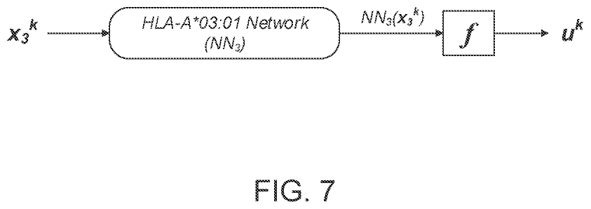

FIG. 7 illustrates generating a presentation likelihood for peptide p^{k} in association with MHC allele h=3 using an example network model NN₃(⋅). As shown in FIG. 7, the network model NN₃(⋅) receives the allele-interacting variables xₖ³ for MHC allele h=3 and generates the output NN₃(xₖ³). The output is mapped by function ƒ(⋅) to generate the estimated presentation likelihood uₖ.

### VIII.B.2. Per-Allele with Allele-Noninteracting Variables

In one implementation, the training module 316 incorporates allele-noninteracting variables and models the estimated presentation likelihood uₖ for peptide p^{k} by:

uₖ^{h}=Pr(p^{k} presented)=ƒ(g_{w}(w^{k};θ_{w})+gₕ(xₕⁱ;θₕ)), (8)

where w^{k} denotes the encoded allele-noninteracting variables for peptide p^{k}, g_{w}(⋅) is a function for the allele-noninteracting variables w^{k} based on a set of parameters θ_{w} determined for the allele-noninteracting variables. Specifically, the values for the set of parameters θₕ for each MHC allele h and the set of parameters θ_{w} for allele-noninteracting variables can be determined by minimizing the loss function with respect to θₕ and θ_{w}, where i is each instance in the subset S of training data 170 generated from cells expressing single MHC alleles.

The output of the dependency function g_{w}(w^{k};θ_{w}) represents a dependency score for the allele noninteracting variables indicating whether the peptide p^{k} will be presented by one or more MHC alleles based on the impact of allele noninteracting variables. For example, the dependency score for the allele noninteracting variables may have a high value if the peptide p^{k} is associated with a C-terminal flanking sequence that is known to positively impact presentation of the peptide p^{k}, and may have a low value if the peptide p is associated with a C-terminal flanking sequence that is known to negatively impact presentation of the peptide p^{k}.

According to equation (8), the per-allele likelihood that a peptide sequence p^{k} will be presented by a MHC allele h can be generated by applying the function gₕ(⋅) for the MHC allele h to the encoded version of the peptide sequence p^{k} to generate the corresponding dependency score for allele interacting variables. The function g_{w}(⋅) for the allele noninteracting variables are also applied to the encoded version of the allele noninteracting variables to generate the dependency score for the allele noninteracting variables. Both scores are combined, and the combined score is transformed by the transformation function ƒ(⋅) to generate a per-allele likelihood that the peptide sequence p^{k} will be presented by the MHC allele h.

Alternatively, the training module 316 may include allele-noninteracting variables w^{k} in the prediction by adding the allele-noninteracting variables w^{k} to the allele-interacting variables xₕ^{k} in equation (2). Thus, the presentation likelihood can be given by:

uₖ^{h}=Pr(p^{k} presented;allele h)=ƒ(gₕ([xₕ^{k}w^{k}];θₕ)). (9)

### VIII.B.3 Dependency Functions for Allele-Noninteracting Variables

Similarly to the dependency function gₕ(⋅) for allele-interacting variables, the dependency function g_{w}(⋅) for allele noninteracting variables may be an affine function or a network function in which a separate network model is associated with allele-noninteracting variables w^{k}.

Specifically, the dependency function g_{w}(⋅) is an affine function given by:

g_{w}(w^{k};θ_{w})=w^{k}·θ_{w}.

that linearly combines the allele-noninteracting variables in w^{k} with a corresponding parameter in the set of parameters θ_{w}.

The dependency function g_{w}(⋅) may also be a network function given by:

g_{w}(w^{k};θ_{w})=NN_{w}(w^{k};θ_{w}).

represented by a network model NN_{w}(⋅) having an associated parameter in the set of parameters θ_{w}. The network function may also include one or more network models each taking different allele noninteracting variables as input.

In another instance, the dependency function g_{w}(⋅) for the allele-noninteracting variables can be given by:

g_{w}(w^{k};θ_{w})=g′_{w}(w^{k};θ′_{w})+h(m^{k};θ_{w}^{m}), (10)

where g′_{w}(w^{k};θ′_{w}) is the affine function, the network function with the set of allele noninteracting parameters θ′_{w}, or the like, m^{k} is the mRNA quantification measurement for peptide p^{k}, h(⋅) is a function transforming the quantification measurement, and θ_{w}^{m} is a parameter in the set of parameters for allele noninteracting variables that is combined with the mRNA quantification measurement to generate a dependency score for the mRNA quantification measurement. In one particular embodiment referred throughout the remainder of the specification, h(⋅) is the log function, however in practice h(⋅) may be any one of a variety of different functions.

In yet another instance, the dependency function g_{w}(⋅) for the allele-noninteracting variables can be given by:

g_{w}(w^{k};θ_{w})=g′_{w}(w^{k};θ′w)+θ_{w}^{o}·o^{k}, (11)

where g′_{w}(w^{k};θ′_{w}) is the affine function, the network function with the set of allele noninteracting parameters θ′_{w}, or the like, o^{k} is the indicator vector described in Section VII.C.2 representing proteins and isoforms in the human proteome for peptide p^{k}, and θ_{w}^{o} is a set of parameters in the set of parameters for allele noninteracting variables that is combined with the indicator vector. In one variation, when the dimensionality of o^{k} and the set of parameters θ_{w}^{o} are significantly high, a parameter regularization term, such as λ·∥θ_{w}^{o}∥, where ∥⋅∥ represents L1 norm, L2 norm, a combination, or the like, can be added to the loss function when determining the value of the parameters. The optimal value of the hyperparameter k can be determined through appropriate methods.

In yet another instance, the dependency function g_{w}(⋅) for the allele-noninteracting variables can be given by:

$${g}_{w} ( {w}^{k} ; {θ}_{w} ) = {g}_{w}^{′} ( {w}^{k} ; {θ}_{w}^{′} ) + {\sum }_{l = 1}^{L} ( gene ( {p}^{k} = l ) ) \cdot {θ}_{w}^{l} , \qquad ( 12 )$$

where g′_{w}(w^{k};θ′_{w}) is the affine function, the network function with the set of allele noninteracting parameters θ′_{w}, or the like, [symbol] (gene(p^{k}=1)) is the indicator function that equals to 1 if peptide p^{k} is from source gene l as described above in reference to allele noninteracting variables, and θ_{w}^{l} is a parameter indicating “antigenicity” of source gene l. In one variation, when L is significantly high, and thus, the number of parameters θ_{w}^{l=1, 2, . . . , L} are significantly high, a parameter regularization term, such as λ·∥θ_{w}^{l}∥, where ∥⋅∥ represents L1 norm, L2 norm, a combination, or the like, can be added to the loss function when determining the value of the parameters. The optimal value of the hyperparameter λ can be determined through appropriate methods.

In yet another instance, the dependency function g_{w}(⋅) for the allele-noninteracting variables can be given by:

$${g}_{w} ( {w}^{k} ; {θ}_{w} ) = {g}_{w}^{′} ( {w}^{k} ; {θ}_{w}^{′} ) + {\sum }_{m = 1}^{M} {\sum }_{l = 1}^{L} ( gene ( {p}^{k} ) = l , tissue ( {p}^{k} ) = m ) \cdot {θ}_{w}^{lm} , \qquad ( 12 b )$$

where g′_{w}(w^{k}; θ′_{w}) is the affine function, the network function with the set of allele noninteracting parameters θ′_{w}, or the like, [symbol] (gene(p^{k})=l, tissue(p^{k})=m) is the indicator function that equals to 1 if peptide p^{k} is from source gene l and if peptide p^{k} is from tissue type m as described above in reference to allele noninteracting variables, and θ_{w}^{lm} is a parameter indicating antigenicity of the combination of source gene l and tissue type m. Specifically, the antigenicity of gene l for tissue type m may denote the residual propensity for cells of tissue type m to present peptides from gene l after controlling for RNA expression and peptide sequence context.

In one variation, when L or A is significantly high, and thus, the number of parameters θ_{w}^{lm=1, 2, . . . , LM} are significantly high, a parameter regularization term, such as as λ·∥θ_{w}^{lm}∥, where ∥⋅∥ represents L1 norm, L2 norm, a combination, or the like, can be added to the loss function when determining the value of the parameters. The optimal value of the hyperparameter λ can be determined through appropriate methods. In another variation, a parameter regularization term can be added to the loss function when determining the value of the parameters, such that the coefficients for the same source gene do not significantly differ between tissue types. For example, a penalization term such as:

$$λ \cdot {\sum }_{l = 1}^{L} \sqrt{{\sum }_{m = 1}^{M} {( {θ}_{w}^{l m} - \overline{{θ}_{w}^{l}} )}^{2}}$$

where θ̅_̅{̅w̅}̅^̅{̅l̅}̅ is the average antigenicity across tissue types for source gene l, may penalize the standard deviation of antigenicity across different tissue types in the loss function.

In practice, the additional terms of any of equations (10), (11), (12a) and (12b) may be combined to generate the dependency function g_{w}(⋅) for allele noninteracting variables. For example, the term h(⋅) indicating mRNA quantification measurement in equation (10) and the term indicating source gene antigenicity in equation (12) may be summed together along with any other affine or network function to generate the dependency function for allele noninteracting variables.

Returning to equation (8), as an example, the likelihood that peptide p^{k} will be presented by MHC allele h=3, among m=4 different identified MHC alleles using the affine transformation functions gₕ(⋅),g_{w}(⋅), can be generated by:

uₖ³=ƒ(w^{k}·0_{w}+xₖ³·θ₃),

where wₖ are the identified allele-noninteracting variables for peptide p^{k} and θ_{w} are the set of parameters determined for the allele-noninteracting variables.

As another example, the likelihood that peptide p^{k} will be presented by MHC allele h=3, among m=4 different identified MHC alleles using the network transformation functions gₕ(⋅), g_{w}(⋅), can be generated by:

uₖ³=ƒ(NN_{w}(w^{k};θ_{w})+NN₃(xₖ³;θ₃))

where w^{k} are the identified allele-interacting variables for peptide p^{k}, and θ_{w} are the set of parameters determined for allele-noninteracting variables.

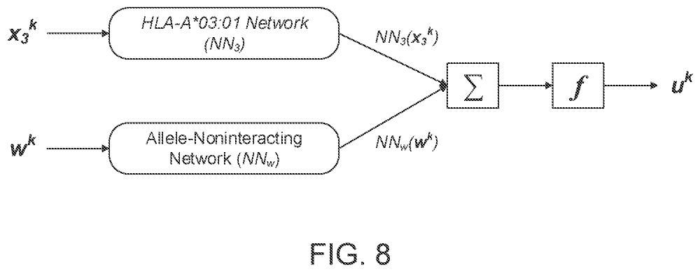

FIG. 8 illustrates generating a presentation likelihood for peptide p^{k} in association with MHC allele h=3 using example network models NN₃(⋅) and NN_{w}(⋅). As shown in FIG. 8, the network model NN₃(⋅) receives the allele-interacting variables xₖ³ for MHC allele h=3 and generates the output NN₃(x₃^{k}). The network model NN_{w}(⋅) receives the allele-noninteracting variables w^{k} for peptide p^{k} and generates the output NN_{w}(w^{k}). The outputs are combined and mapped by function ƒ(⋅) to generate the estimated presentation likelihood uₖ.

### VII.C. Multiple-Allele Models

The training module 316 may also construct the presentation models to predict presentation likelihoods of peptides in a multiple-allele setting where two or more MHC alleles are present. In this case, the training module 316 may train the presentation models based on data instances S in the training data 170 generated from cells expressing single MHC alleles, cells expressing multiple MHC alleles, or a combination thereof.

### VIII.C.1. Example 1: Maximum of Per-Allele Models

In one implementation, the training module 316 models the estimated presentation likelihood uₖ for peptide p^{k} in association with a set of multiple MHC alleles H as a function of the presentation likelihoods uₖ^{h}^{_{∈H}}H determined for each of the MHC alleles h in the set H determined based on cells expressing single-alleles, as described above in conjunction with equations (2)-(11). Specifically, the presentation likelihood Ilk can be any function of uₖ^{h}^{_{∈H}}. In one implementation, as shown in equation (12), the function is the maximum function, and the presentation likelihood uₖ can be determined as the maximum of the presentation likelihoods for each MHC allele h in the set H.

uₖ=Pr(p^{k} presented;alleles H)=max(uₖ^{h}^{_{∈H}})

### VIII.C.2. Example 2.1: Function-of-Sums Models

In one implementation, the training module 316 models the estimated presentation likelihood uₖ for peptide p^{k} by:

$${u}_{k} = Pr ( {p}^{k} presented ) = f ( {\sum }_{h = 1}^{m} {a}_{h}^{k} \cdot {g}_{h} ( {x}_{h}^{k} ; {θ}_{h} ) ) , \qquad ( 13 )$$

where elements aₕ^{k} are 1 for the multiple MHC alleles H associated with peptide sequence p^{k} and xₕ^{k} denotes the encoded allele-interacting variables for peptide p^{k} and the corresponding MHC alleles. The values for the set of parameters θₕ for each MHC allele h can be determined by minimizing the loss function with respect to θₕ, where i is each instance in the subset S of training data 170 generated from cells expressing single MHC alleles and/or cells expressing multiple MHC alleles. The dependency function gi may be in the form of any of the dependency functions gₕ introduced above in sections VIII.B.1.

According to equation (13), the presentation likelihood that a peptide sequence p^{k} will be presented by one or more MHC alleles h can be generated by applying the dependency function gₕ(⋅) to the encoded version of the peptide sequence p^{k} for each of the MHC alleles H to generate the corresponding score for the allele interacting variables. The scores for each MHC allele h are combined, and transformed by the transformation function ƒ(⋅) to generate the presentation likelihood that peptide sequence p^{k} will be presented by the set of MHC alleles H.

The presentation model of equation (13) is different from the per-allele model of equation (2), in that the number of associated alleles for each peptide p^{k} can be greater than 1. In other words, more than one element in aₕ^{k} can have values of 1 for the multiple MHC alleles H associated with peptide sequence p^{k}.

As an example, the likelihood that peptide p^{k} will be presented by MHC alleles h=2, h=3, among m=4 different identified MHC alleles using the affine transformation functions gₕ(⋅), can be generated by:

uₖ=ƒ(x₂^{k}·θ₂+x₃^{k}·θ₃),

where x₂^{k}, x₃^{k} are the identified allele-interacting variables for MHC alleles h=2, h=3, and θ₂, θ₃ are the set of parameters determined for MHC alleles h=2, h=3.

As another example, the likelihood that peptide p^{k} will be presented by MHC alleles h=2, h=3, among m=4 different identified MHC alleles using the network transformation functions gₕ(⋅), g_{w}(⋅), can be generated by:

uₖ=ƒ(NN₂(x₂^{k};θ₂)+NN₃(x₃^{k};θ₃)),

where NN₂(⋅), NN₃(⋅) are the identified network models for MHC alleles h=2, h=3, and θ₂,θ₃ are the set of parameters determined for MHC alleles h=2, h=3.

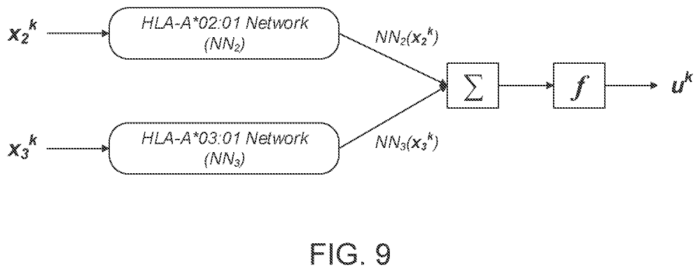

FIG. 9 illustrates generating a presentation likelihood for peptide p^{k} in association with MHC alleles h=2, h=3 using example network models NN₂(⋅) and NN₃(⋅). As shown in FIG. 9, the network model NN₂(⋅) receives the allele-interacting variables x₂^{k} for MHC allele h=2 and generates the output NN₂(x₂^{k}) and the network model NN₃(⋅) receives the allele-interacting variables x₃^{k} for MHC allele h=3 and generates the output NN₃(x₃^{k}). The outputs are combined and mapped by function ƒ(⋅) to generate the estimated presentation likelihood uₖ.

### VIII.C.3. Example 2.2: Function-of-Sums Models with Allele-Noninteracting Variables

In one implementation, the training module 316 incorporates allele-noninteracting variables and models the estimated presentation likelihood uₖ for peptide p^{k} by:

$${u}_{k} = Pr ( {p}^{k} presented ) = f ( {g}_{w} ( {w}^{k} ; {θ}_{w} ) + {\sum }_{h = 1}^{m} {a}_{h}^{k} \cdot {g}_{h} ( {x}_{h}^{k} ; {θ}_{h} ) ) , \qquad ( 14 )$$

where w^{k} denotes the encoded allele-noninteracting variables for peptide p^{k}. Specifically, the values for the set of parameters θₕ for each MHC allele h and the set of parameters θ_{w} for allele-noninteracting variables can be determined by minimizing the loss function with respect to θₕ and θ_{w}, where i is each instance in the subset S of training data 170 generated from cells expressing single MHC alleles and/or cells expressing multiple MHC alleles. The dependency function g_{w} may be in the form of any of the dependency functions g_{w} introduced above in sections VIII.B.3.

Thus, according to equation (14), the presentation likelihood that a peptide sequence p^{k} will be presented by one or more MHC alleles H can be generated by applying the function gₕ(⋅) to the encoded version of the peptide sequence p^{k} for each of the MHC alleles H to generate the corresponding dependency score for allele interacting variables for each MHC allele h. The function g_{w}(⋅) for the allele noninteracting variables is also applied to the encoded version of the allele noninteracting variables to generate the dependency score for the allele noninteracting variables. The scores are combined, and the combined score is transformed by the transformation function ƒ(⋅) to generate the presentation likelihood that peptide sequence pₖ will be presented by the MHC alleles H.

In the presentation model of equation (14), the number of associated alleles for each peptide p^{k} can be greater than 1. In other words, more than one element in aₕ^{k} can have values of 1 for the multiple MHC alleles H associated with peptide sequence p^{k}.

As an example, the likelihood that peptide p^{k} will be presented by MHC alleles h=2, h=3, among m=4 different identified MHC alleles using the affine transformation functions gₕ(⋅), g_{w}(⋅), can be generated by:

uₖ=ƒ(NN_{w}(w^{k};θ_{w})+NN₂(x₂^{k};θ₂)NN₃(x₃^{k};θ₃))

where w^{k} are the identified allele-noninteracting variables for peptide p^{k}, and θ_{w} are the set of parameters determined for the allele-noninteracting variables.

As another example, the likelihood that peptide p^{k} will be presented by MHC alleles h=2, h=3, among m=4 different identified MHC alleles using the network transformation functions gₕ(⋅), g_{w}(⋅), can be generated by:

uₖ=ƒ(NN_{w}(w^{k};θ_{w})+NN₂(x₂^{k};θ₂)+NN₃(x₃^{k};θ₃))

where w^{k} are the identified allele-interacting variables for peptide p^{k}, and θ_{w} are the set of parameters determined for allele-noninteracting variables.

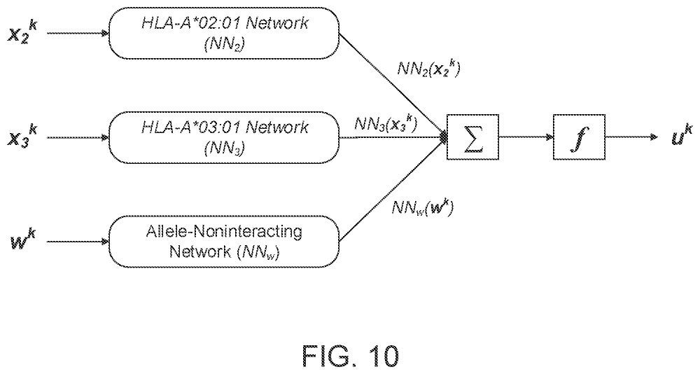

FIG. 10 illustrates generating a presentation likelihood for peptide p^{k} in association with MHC alleles h=2, h=3 using example network models NN₂(⋅), NN₃(⋅), and NN_{w}(⋅). As shown in FIG. 10, the network model NN₂(⋅) receives the allele-interacting variables x₂^{k} for MHC allele h=2 and generates the output NN₂(x₂^{k}). The network model NN₃(⋅) receives the allele-interacting variables x₃^{k} for MHC allele h=3 and generates the output NN₃(x₃^{k}). The network model NN_{w}(⋅) receives the allele-noninteracting variables w^{k} for peptide p^{k} and generates the output NN_{w}(w^{k}). The outputs are combined and mapped by function ƒ(⋅) to generate the estimated presentation likelihood uₖ.

Alternatively, the training module 316 may include allele-noninteracting variables w^{k} in the prediction by adding the allele-noninteracting variables w^{k} to the allele-interacting variables xₕ^{k} in equation (15). Thus, the presentation likelihood can be given by:

$${u}_{k} = Pr ( {p}^{k} presented ) = f ( {\sum }_{h = 1}^{m} {a}_{h}^{k} \cdot {g}_{h} ( [ {x}_{h}^{k} {w}^{k} ] ; {θ}_{h} ) ) . \qquad ( 15 )$$

### VIII.C.4. Example 3.1: Models Using Implicit Per-Allele Likelihoods

In another implementation, the training module 316 models the estimated presentation likelihood uₖ for peptide p^{k} by:

uₖ=Pr(p^{k} presented)=r(s(v=[a₁^{k}·u′ₖ¹(θ) . . . aₘ^{k}·u′ₖ^{m}(θ)])), (16)

where elements aₕ^{k} are 1 for the multiple MHC alleles h∈H associated with peptide sequence p^{k}, u′ₖ^{h} is an implicit per-allele presentation likelihood for MHC allele h, vector v is a vector in which element vₕ corresponds to aₕ^{k}·u′ₖ^{h}, s(⋅) is a function mapping the elements of v, and r(⋅) is a clipping function that clips the value of the input into a given range. As described below in more detail, s(⋅) may be the summation function or the second-order function, but it is appreciated that in other embodiments, s(⋅) can be any function such as the maximum function. The values for the set of parameters θ for the implicit per-allele likelihoods can be determined by minimizing the loss function with respect to θ, where i is each instance in the subset S of training data 170 generated from cells expressing single MHC alleles and/or cells expressing multiple MHC alleles.

The presentation likelihood in the presentation model of equation (17) is modeled as a function of implicit per-allele presentation likelihoods u′k^{h} that each correspond to the likelihood peptide p^{k} will be presented by an individual MHC allele h. The implicit per-allele likelihood is distinct from the per-allele presentation likelihood of section VIII.B in that the parameters for implicit per-allele likelihoods can be learned from multiple allele settings, in which direct association between a presented peptide and the corresponding MHC allele is unknown, in addition to single-allele settings. Thus, in a multiple-allele setting, the presentation model can estimate not only whether peptide p^{k} will be presented by a set of MHC alleles H as a whole, but can also provide individual likelihoods u′ₖ^{h}^{_{∈H}} that indicate which MHC allele h most likely presented peptide p^{k}. An advantage of this is that the presentation model can generate the implicit likelihoods without training data for cells expressing single MHC alleles.

In one particular implementation referred throughout the remainder of the specification, r(⋅) is a function having the range [0, 1]. For example, r(⋅) may be the clip function:

r(z)=min(max(z,0),1),

where the minimum value between z and 1 is chosen as the presentation likelihood uₖ. In another implementation, r(⋅) is the hyperbolic tangent function given by:

r(z)=tan h(z)

when the values for the domain z is equal to or greater than 0.

### VIII.C.5. Example 3.2: Sum-of-Functions Model

In one particular implementation, s(⋅) is a summation function, and the presentation likelihood is given by summing the implicit per-allele presentation likelihoods:

$${u}_{k} = Pr ( {p}^{k} presented ) = r ( {\sum }_{h = 1}^{m} {a}_{h}^{k} \cdot {u}_{k}^{′ h} ( θ ) ) . \qquad ( 17 )$$

In one implementation, the implicit per-allele presentation likelihood for MHC allele h is generated by:

u′ₖ^{h}=ƒ(gₕ(xₕ^{k};θₕ)), (18)

such that the presentation likelihood is estimated by:

$${u}_{k} = Pr ( {p}^{k} presented ) = r ( {\sum }_{h = 1}^{m} {a}_{h}^{k} \cdot f ( {g}_{h} ( {x}_{h}^{k} ; {θ}_{h} ) ) ) . \qquad ( 19 )$$

According to equation (19), the presentation likelihood that a peptide sequence p^{k} will be presented by one or more MHC alleles H can be generated by applying the function gₕ(∩) to the encoded version of the peptide sequence p^{k} for each of the MHC alleles H to generate the corresponding dependency score for allele interacting variables. Each dependency score is first transformed by the function ƒ(⋅) to generate implicit per-allele presentation likelihoods u′ₖ^{h}. The per-allele likelihoods u′ₖ^{h} are combined, and the clipping function may be applied to the combined likelihoods to clip the values into a range [0, 1] to generate the presentation likelihood that peptide sequence p^{k} will be presented by the set of MHC alleles H. The dependency function gₕ may be in the form of any of the dependency functions gₕ introduced above in sections VIII.B.1.

As an example, the likelihood that peptide p^{k} will be presented by MHC alleles h=2, h=3, among m=4 different identified MHC alleles using the affine transformation functions gₕ(⋅), can be generated by:

uₖ=r(ƒ(x₂^{k};θ₂)+ƒ(x₃^{k};θ₃)),

where x₂^{k}, x₃^{k} are the identified allele-interacting variables for MHC alleles h=2, h=3, and θ₂, θ₃ are the set of parameters determined for MHC alleles h=2, h=3.

As another example, the likelihood that peptide p^{k} will be presented by MHC alleles h=2, h=3, among m=4 different identified MHC alleles using the network transformation functions gₕ(⋅), g_{w}(⋅), can be generated by:

uₖ=r(ƒ(NN₂(x₂^{k};θ₂))+ƒ(NN₃(x₃^{k};θ₃))),

where NN₂(⋅), NN₃(⋅) are the identified network models for MHC alleles h=2, h=3, and θ₂,θ₃ are the set of parameters determined for MHC alleles h=2, h=3.

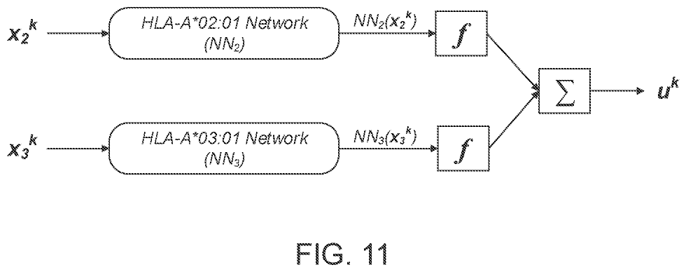

FIG. 11 illustrates generating a presentation likelihood for peptide p^{k} in association with MHC alleles h=2, h=3 using example network models NN₂(⋅) and NN₃(⋅). As shown in FIG. 9, the network model NN₂(⋅) receives the allele-interacting variables x₂^{k} for MHC allele h=2 and generates the output NN₂(x₂^{k}) and the network model NN₃(x₃^{k}) receives the allele-interacting variables x₃^{k} for MHC allele h=3 and generates the output NN₃(x₃^{k}). Each output is mapped by function ƒ(⋅) and combined to generate the estimated presentation likelihood uₖ.

In another implementation, when the predictions are made for the log of mass spectrometry ion currents, r(⋅) is the log function and ƒ(⋅) is the exponential function.

### VIII.C.6. Example 3.3: Sum-of-Functions Models with Allele-Noninteracting Variables

In one implementation, the implicit per-allele presentation likelihood for MHC allele h is generated by:

u′ₖ^{h}=ƒ(gₕ(xₕ^{k};θₕ)+g_{w}(w^{k};θ_{w})). (20)

such that the presentation likelihood is generated by:

$${u}_{k} = Pr ( {p}^{k} presented ) = r ( {\sum }_{h = 1}^{m} {a}_{h}^{k} \cdot f ( {g}_{w} ( {w}^{k} ; {θ}_{w} ) + {g}_{h} ( {x}_{h}^{k} ; {θ}_{h} ) ) ) . \qquad ( 21 )$$

to incorporate the impact of allele noninteracting variables on peptide presentation.

According to equation (21), the presentation likelihood that a peptide sequence p^{k} will be presented by one or more MHC alleles H can be generated by applying the function gₕ(⋅) to the encoded version of the peptide sequence p^{k} for each of the MHC alleles H to generate the corresponding dependency score for allele interacting variables for each MHC allele h. The function g_{w}(⋅) for the allele noninteracting variables is also applied to the encoded version of the allele noninteracting variables to generate the dependency score for the allele noninteracting variables. The score for the allele noninteracting variables are combined to each of the dependency scores for the allele interacting variables. Each of the combined scores are transformed by the function ƒ(⋅) to generate the implicit per-allele presentation likelihoods. The implicit likelihoods are combined, and the clipping function may be applied to the combined outputs to clip the values into a range [0,1] to generate the presentation likelihood that peptide sequence p^{k} will be presented by the MHC alleles H. The dependency function g_{w} may be in the form of any of the dependency functions g_{w} introduced above in sections VIII.B.3.

As an example, the likelihood that peptide p^{k} will be presented by MHC alleles h=2, h=3, among m=4 different identified MHC alleles using the affine transformation functions gₕ(⋅), g_{w}(⋅), can be generated by:

uₖ=r(ƒ(w^{k}·θ_{w}+x₂^{k}·θ₂)+ƒ(w^{k}·θ_{w}+x₃^{k}·θ₃)),

where w^{k} are the identified allele-noninteracting variables for peptide p^{k}, and θ_{w} are the set of parameters determined for the allele-noninteracting variables.

As another example, the likelihood that peptide p^{k} will be presented by MHC alleles h=2, h=3, among m=4 different identified MHC alleles using the network transformation functions gₕ(⋅), g_{w}(⋅), can be generated by:

uₖ=r(ƒ(NN_{w}(w^{k};θ_{w})+NN₂(x₂^{k};θ₂))+ƒ(NN_{w}(w^{k};θ_{w})+NN₃(x₃^{k};θ₃)))

where w^{k} are the identified allele-interacting variables for peptide p^{k}, and θ_{w} are the set of parameters determined for allele-noninteracting variables.

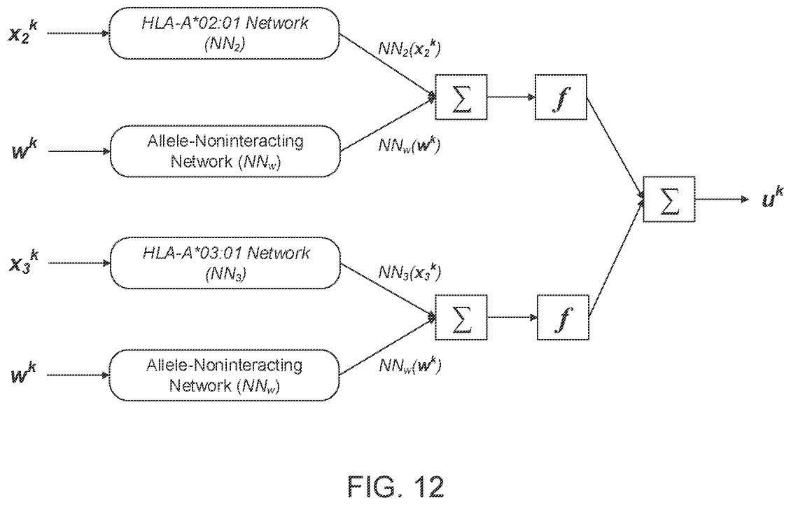

FIG. 12 illustrates generating a presentation likelihood for peptide p^{k} in association with MHC alleles h=2, h=3 using example network models NN₂(⋅), NN₃(⋅), and NN_{w}(⋅). As shown in FIG. 12, the network model NN₂(⋅) receives the allele-interacting variables x₂^{k} for MHC allele h=2 and generates the output NN₂(x₂^{k}). The network model NN_{w}(⋅) receives the allele-noninteracting variables w^{k} for peptide p^{k} and generates the output NN_{w}(w^{k}). The outputs are combined and mapped by function ƒ(⋅). The network model NN₃(⋅) receives the allele-interacting variables x₃^{k} for MHC allele h=3 and generates the output NN₃(x₃^{k}), which is again combined with the output NN_{w}(w^{k}) of the same network model NN_{w}(⋅) and mapped by function ƒ(⋅). Both outputs are combined to generate the estimated presentation likelihood uₖ.

In another implementation, the implicit per-allele presentation likelihood for MHC allele h is generated by:

u′ₖ^{h}=ƒ(gₕ([xₕ^{k} w^{k}];θₕ)). (22)

such that the presentation likelihood is generated by:

$${u}_{k} = Pr ( {p}^{k} presented ) = r ( {\sum }_{h = 1}^{m} {a}_{h}^{k} \cdot f ( {g}_{h} ( [ {x}_{h}^{k} {w}^{k} ] ; {θ}_{h} ) ) ) .$$

### VIII.C.7. Example 4: Second Order Models

In one implementation, s(⋅) is a second-order function, and the estimated presentation likelihood uₖ for peptide p^{k} is given by:

$${u}_{k} = Pr ( {p}^{k} presented ) = {\sum }_{h = 1}^{m} {a}_{h}^{k} \cdot {{u}_{k}^{′}}^{h} ( θ ) - {\sum }_{h = 1}^{m} {\sum }_{j < h}^{ } {a}_{h}^{k} \cdot {a}_{j}^{k} \cdot {u}_{k}^{′ h} ( θ ) \cdot {u}_{k}^{′ j} ( θ ) \qquad ( 23 )$$

where elements u′ₖ^{h} are the implicit per-allele presentation likelihood for MHC allele h. The values for the set of parameters θ for the implicit per-allele likelihoods can be determined by minimizing the loss function with respect to θ, where i is each instance in the subset S of training data 170 generated from cells expressing single MHC alleles and/or cells expressing multiple MHC alleles. The implicit per-allele presentation likelihoods may be in any form shown in equations (18), (20), and (22) described above.

In one aspect, the model of equation (23) may imply that there exists a possibility peptide p^{k} will be presented by two MHC alleles simultaneously, in which the presentation by two HLA alleles is statistically independent.

According to equation (23), the presentation likelihood that a peptide sequence p^{k} will be presented by one or more MHC alleles H can be generated by combining the implicit per-allele presentation likelihoods and subtracting the likelihood that each pair of MHC alleles will simultaneously present the peptide p from the summation to generate the presentation likelihood that peptide sequence p will be presented by the MHC alleles H.

As an example, the likelihood that peptide p^{k} will be presented by HLA alleles h=2, h=3, among m=4 different identified HLA alleles using the affine transformation functions gₕ(⋅), can be generated by:

uₖ=ƒ(x₂^{k}·θ₂)+ƒ(x₃^{k}·θ₃)−ƒ(x₃^{k}·θ₂)·ƒ(x₃^{k}·θ₃),

where x₂^{k}, x₃^{k} are the identified allele-interacting variables for HLA alleles h=2, h=3, and θ₂,θ₃ are the set of parameters determined for HLA alleles h=2, h=3.

As another example, the likelihood that peptide p will be presented by HLA alleles h=2, h=3, among m=4 different identified HLA alleles using the network transformation functions gₕ(⋅), g_{w}(⋅), can be generated by:

uₖ=ƒ(NN₂(x₂^{k};θ₂))+ƒ(NN₃(x₃^{k};θ₃))−ƒ(NN₂(x₂^{k};θ₂))·ƒ(NN₃(x₃^{k};θ₃)),

where NN₂(⋅), NN₃(⋅) are the identified network models for HLA alleles h=2, h=3, and θ₂,θ₃ are the set of parameters determined for HLA alleles h=2, h=3.

### IX. Example 5: Prediction Module

The prediction module 320 receives sequence data and selects candidate neoantigens in the sequence data using the presentation models. Specifically, the sequence data may be DNA sequences, RNA sequences, and/or protein sequences extracted from tumor tissue cells of patients. The prediction module 320 processes the sequence data into a plurality of peptide sequences p^{k} having 8-15 amino acids for MHC-I or 6-30 amino acids for MC-II. For example, the prediction module 320 may process the given sequence “IEFROEIFJEF into three peptide sequences having 9 amino acids “IEFROEIFJ,” “EFROEIFJE,” and “FROEIFJEF.” In one embodiment, the prediction module 320 may identify candidate neoantigens that are mutated peptide sequences by comparing sequence data extracted from normal tissue cells of a patient with the sequence data extracted from tumor tissue cells of the patient to identify portions containing one or more mutations.

The prediction module 320 applies one or more of the presentation models to the processed peptide sequences to estimate presentation likelihoods of the peptide sequences. Specifically, the prediction module 320 may select one or more candidate neoantigen peptide sequences that are likely to be presented on tumor HLA molecules by applying the presentation models to the candidate neoantigens. In one implementation, the prediction module 320 selects candidate neoantigen sequences that have estimated presentation likelihoods above a predetermined threshold. In another implementation, the presentation model selects the v candidate neoantigen sequences that have the highest estimated presentation likelihoods (where v is generally the maximum number of epitopes that can be delivered in a vaccine). A vaccine including the selected candidate neoantigens for a given patient can be injected into the patient to induce immune responses.

### X. Example 6: Patient Selection Module

The patient selection module 324 selects a subset of patients for vaccine treatment and/or T-cell therapy based on whether the patients satisfy inclusion criteria. In one embodiment, the inclusion criteria is determined based on the presentation likelihoods of patient neoantigen candidates as generated by the presentation models. By adjusting the inclusion criteria, the patient selection module 324 can adjust the number of patients that will receive the vaccine and/or T-cell therapy based on his or her presentation likelihoods of neoantigen candidates. Specifically, a stringent inclusion criteria results in a fewer number of patients that will be treated with the vaccine and/or T-cell therapy, but may result in a higher proportion of vaccine and/or T-cell therapy-treated patients that receive effective treatment (e.g., 1 or more tumor-specific neoantigens (TSNA) and/or 1 or more neoantigen-responsive T-cells). On the other hand, a lenient inclusion criteria results in a higher number of patients that will be treated with the vaccine and/or with T-cell therapy, but may result in a lower proportion of vaccine and/or T-cell therapy-treated patients that receive effective treatment. The patient selection module 324 modifies the inclusion criteria based on the desired balance between target proportion of patients that will receive treatment and proportion of patients that receive effective treatment.

In some embodiments, inclusion criteria for selection of patients to receive vaccine treatment are the same as inclusion criteria for selection of patients to receive T-cell therapy. However, in alternative embodiments, inclusion criteria for selection of patients to receive vaccine treatment may differ from inclusion criteria for selection of patients to receive T-cell therapy. The following Sections X.A and X.B discuss inclusion criteria for selection of patients to receive vaccine treatment and inclusion criteria for selection of patients to receive T-cell therapy, respectively.

### X.A. Patient Selection for Vaccine Treatment

In one embodiment, patients are associated with a corresponding treatment subset of v neoantigen candidates that can potentially be included in customized vaccines for the patients with vaccine capacity v. In one embodiment, the treatment subset for a patient are the neoantigen candidates with the highest presentation likelihoods as determined by the presentation models. For example, if a vaccine can include v=20 epitopes, the vaccine can include the treatment subset of each patient that have the highest presentation likelihoods as determined by the presentation model. However, it is appreciated that in other embodiments, the treatment subset for a patient can be determined based on other methods. For example, the treatment subset for a patient may be randomly selected from the set of neoantigen candidates for the patient, or may be determined in part based on current state-of-the-art models that model binding affinity or stability of peptide sequences, or some combination of factors that include presentation likelihoods from the presentation models and affinity or stability information regarding those peptide sequences.

In one embodiment, the patient selection module 324 determines that a patient satisfies the inclusion criteria if the tumor mutation burden of the patient is equal to or above a minimum mutation burden. The tumor mutation burden (TMB) of a patient indicates the total number of nonsynonymous mutations in the tumor exome. In one implementation, the patient selection module 324 may select a patient for vaccine treatment if the absolute number of TMB of the patient is equal to or above a predetermined threshold. In another implementation, the patient selection module 324 may select a patient for vaccine treatment if the TMB of the patient is within a threshold percentile among the TMB's determined for the set of patients.

In another embodiment, the patient selection module 324 determines that a patient satisfies the inclusion criteria if a utility score of the patient based on the treatment subset of the patient is equal to or above a minimum utility score. In one implementation, the utility score is a measure of the estimated number of presented neoantigens from the treatment subset.

The estimated number of presented neoantigens may be predicted by modeling neoantigen presentation as a random variable of one or more probability distributions. In one implementation, the utility score for patient i is the expected number of presented neoantigen candidates from the treatment subset, or some function thereof. As an example, the presentation of each neoantigen can be modeled as a Bemoulli random variable, in which the probability of presentation (success) is given by the presentation likelihood of the neoantigen candidate. Specifically, for a treatment subset Sᵢ of v neoantigen candidates pⁱ¹, pⁱ², . . . , p^{iv} each having the highest presentation likelihoods uᵢ₁, uᵢ₂, . . . , uᵢᵥ, presentation of neoantigen candidate p^{ij} is given by random variable Aᵢⱼ, in which:

P(Aᵢⱼ=1)=uᵢⱼ,P(Aᵢⱼ=0)=1−uᵢⱼ. (24)

The expected number of presented neoantigens is given by the summation of the presentation likelihoods for each neoantigen candidate. In other words, the utility score for patient i can be expressed as:

$${util}_{i} ( {S}_{i} ) = 𝔼 [ {\sum }_{j = 1}^{v} {A}_{ij} ] = {\sum }_{j = 1}^{v} {u}_{ij} . \qquad ( 25 )$$

The patient selection module 324 selects a subset of patients having utility scores equal to or above a minimum utility for vaccine treatment.

In another implementation, the utility score for patient i is the probability that at least a threshold number of neoantigens k will be presented. In one instance, the number of presented neoantigens in the treatment subset S, of neoantigen candidates is modeled as a Poisson Binomial random variable, in which the probabilities of presentation (successes) are given by the presentation likelihoods of each of the epitopes. Specifically, the number of presented neoantigens for patient i can be given by random variable Nᵢ, in which:

$${N}_{i} = {\sum }_{j = 1}^{v} {A}_{ij} ∼ PBD ( {u}_{i 1} , {u}_{i 2} , … , {u}_{iv} ) . \qquad ( 26 )$$

where PBD(⋅) denotes the Poisson Binomial distribution. The probability that at least a threshold number of neoantigens k will be presented is given by the summation of the probabilities that the number of presented neoantigens N, will be equal to or above k. In other words, the utility score for patient i can be expressed as:

$${util}_{i} ( {S}_{i} ) = ℙ [ {N}_{i} \geq k ] = {\sum }_{m = 1}^{k} ℙ [ {N}_{i} = m ] . \qquad ( 27 )$$

The patient selection module 324 selects a subset of patients having the utility score equal to or above a minimum utility for vaccine treatment.

In another implementation, the utility score for patient i is the number of neoantigens in the treatment subset Sᵢ of neoantigen candidates having binding affinity or predicted binding affinity below a fixed threshold (e.g., 500 nM) to one or more of the patient's HLA alleles. In one instance, the fixed threshold is a range from 1000 nM to 10 nM. Optionally, the utility score may count only those neoantigens detected as expressed via RNA-seq.

In another implementation, the utility score for patient i is the number of neoantigens in the treatment subset S, of neoantigen candidates having binding affinity to one or more of that patient's HLA alleles at or below a threshold percentile of binding affinities for random peptides to that HLA allele. In one instance, the threshold percentile is a range from the 10^{th} percentile to the 0.1^{th} percentile. Optionally, the utility score may count only those neoantigens detected as expressed via RNA-seq.

It is appreciated that the examples of generating utility scores illustrated with respect to equations (25) and (27) are merely illustrative, and the patient selection module 324 may use other statistics or probability distributions to generate the utility scores.

### X.B. Patient Selection for T-Cell Therapy

In another embodiment, instead of or in addition to receiving vaccine treatment, patients can receive T-cell therapy. Like vaccine treatment, in embodiments in which a patient receives T-cell therapy, the patient may be associated with a corresponding treatment subset of v neoantigen candidates as described above. This treatment subset of v neoantigen candidates can be used for in vitro identification of T cells from the patient that are responsive to one or more of the v neoantigen candidates. These identified T cells can then be expanded and infused into the patient for customized T-cell therapy.

Patients may be selected to receive T-cell therapy at two different time points. The first point is after the treatment subset of v neoantigen candidates have been predicted for a patient using the models, but before in vitro screening for T cells that are specific to the predicted treatment subset of v neoantigen candidates. The second point is after in vitro screening for T cells that are specific to the predicted treatment subset of v neoantigen candidates.

First, patients may be selected to receive T-cell therapy after the treatment subset of v neoantigen candidates have been predicted for the patient, but before in vitro identification of T-cells from the patient that are specific to the predicted subset of v neoantigen candidates. Specifically, because in vitro screening for neoantigen-specific T-cells from the patient can be expensive, it may be desirable to only select patients to screen for neoantigen-specific T-cells if the patients are likely to have neoantigen-specific T-cells. To select patients before the in vitro T-cell screening step, the same criteria that are used to select patients for vaccine treatment may be used. Specifically, in some embodiments, the patient selection module 324 may select a patient to receive T-cell therapy if the tumor mutation burden of the patient is equal to or above a minimum mutation burden as described above. In another embodiment, the patient selection module 324 may select a patient to receive T-cell therapy if a utility score of the patient based on the treatment subset of v neoantigen candidates for the patient is equal to or above a minimum utility score, as described above.

Second, in addition to or instead of selecting patients to receive T-cell therapy before in vitro identification of T-cells from the patient that are specific to the predicted subset of v neoantigen candidates, patients may also be selected to receive T-cell therapy after in vitro identification of T-cells that are specific to the predicted treatment subset of v neoantigen candidates. Specifically, a patient may be selected to receive T-cell therapy if at least a threshold quantity of neoantigen-specific TCRs are identified for the patient during the in vitro screening of the patient's T-cells for neoantigen recognition. For example, a patient may be selected to receive T-cell therapy only if at least two neoantigen-specific TCRs are identified for the patient, or only if neoantigen-specific TCRs are identified for two distinct neoantigens.

In another embodiment, a patient may be selected to receive T-cell therapy only if at least a threshold quantity of neoantigens of the treatment subset of v neoantigen candidates for the patient are recognized by the patient's TCRs. For example, a patient may be selected to receive T-cell therapy only if at least one neoantigen of the treatment subset of v neoantigen candidates for the patient are recognized by the patient's TCRs. In further embodiments, a patient may be selected to receive T-cell therapy only if at least a threshold quantity of TCRs for the patient are identified as neoantigen-specific to neoantigen peptides of a particular HLA restriction class. For example, a patient may be selected to receive T-cell therapy only if at least one TCR for the patient is identified as neoantigen-specific HLA class I restricted neoantigen peptides.

In even further embodiments, a patient may be selected to receive T-cell therapy only if at least a threshold quantity of neoantigen peptides of a particular HLA restriction class are recognized by the patient's TCRs. For example, a patient may be selected to receive T-cell therapy only if at least one HLA class I restricted neoantigen peptide is recognized by the patient's TCRs. As another example, a patient may be selected to receive T-cell therapy only if at least two HLA class II restricted neoantigen peptides are recognized by the patient's TCRs. Any combination of the above criteria may also be used for selecting patients to receive T-cell therapy after in vitro identification of T-cells that are specific to the predicted treatment subset of v neoantigen candidates for the patient.

### XI. Example 7: Experimentation Results Showing Example Patient Selection Performance

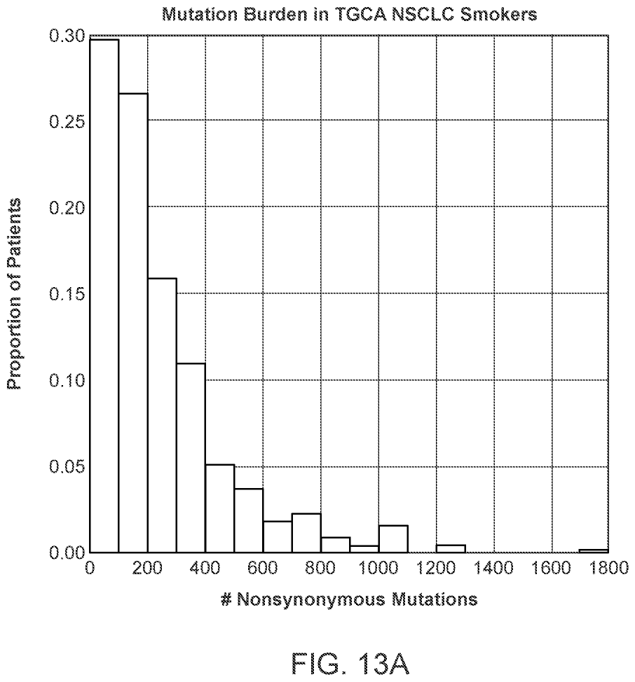

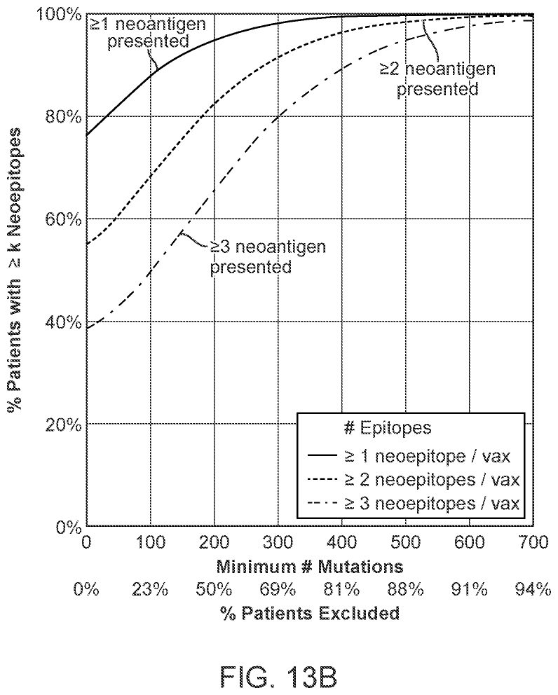

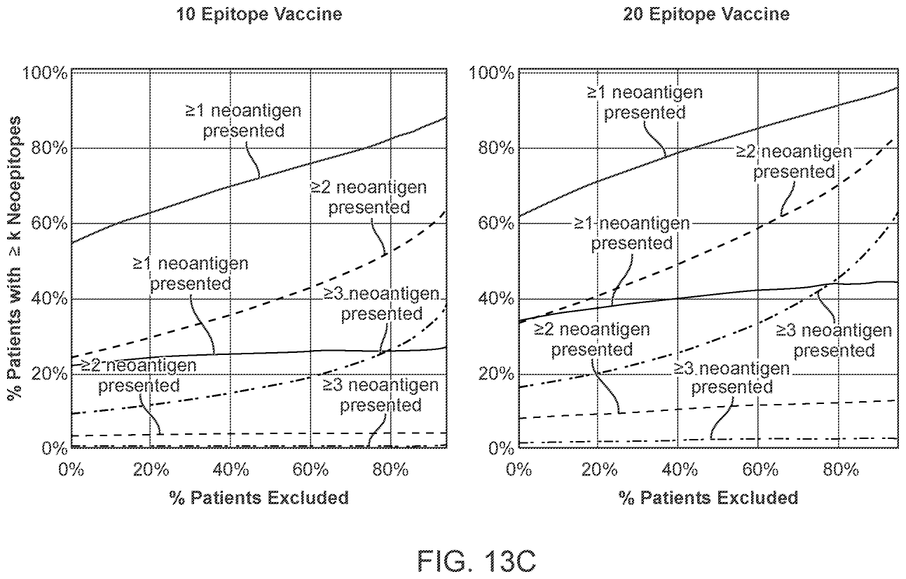

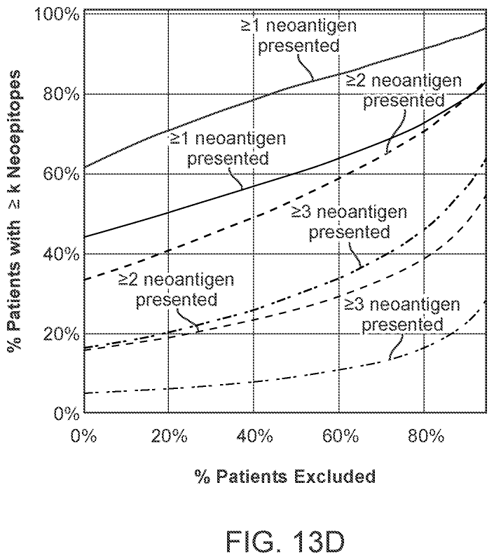

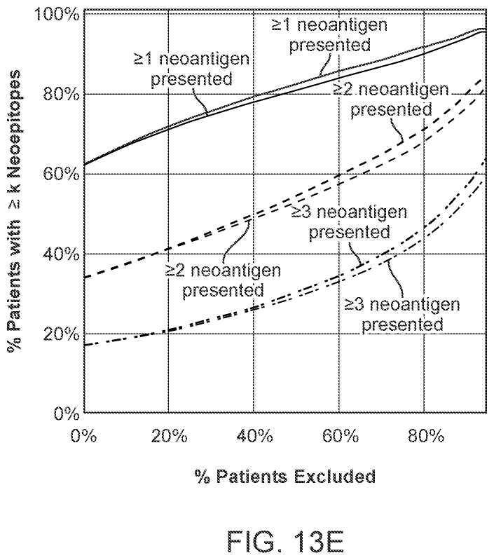

The validity of patient selection methods described in Section X are tested by performing patient selection on a set of simulated patients each associated with a test set of simulated neoantigen candidates, in which a subset of simulated neoantigens is known to be presented in mass spectrometry data. Specifically, each simulated neoantigen candidate in the test set is associated with a label indicating whether the neoantigen was presented in a multiple-allele JY cell line HLA-A*02:01 and HLA-B*07:02 mass spectrometry data set from the Bassani-Stemberg data set (data set “D1”) (data can be found at www.ebi.ac.uk/pride/archive/projects/PXD0000394). As described in more detail below in conjunction with FIG. 13A, a number of neoantigen candidates for the simulated patients are sampled from the human proteome based on the known frequency distribution of mutation burden in non-small cell lung cancer (NSCLC) patients.

Per-allele presentation models for the same HLA alleles are trained using a training set that is a subset of the single-allele HLA-A*02:01 and HLA-B*07:02 mass spectrometry data from the IEDB data set (data set “D2”) (data can be found at http://www.iedb.org/doc/mhc_licand_full.zip). Specifically, the presentation model for each allele was the per-allele model shown in equation (8) that incorporated N-terminal and C-terminal flanking sequences as allele-noninteracting variables, with network dependency functions gₕ(⋅) and g_{w}(⋅), and the expit function ƒ(⋅). The presentation model for allele HLA-A*02:01 generates a presentation likelihood that a given peptide will be presented on allele HLA-A*02:01, given the peptide sequence as an allele-interacting variable, and the N-terminal and C-terminal flanking sequences as allele-noninteracting variables. The presentation model for allele HLA-B*07:02 generates a presentation likelihood that a given peptide will be presented on allele HLA-B*07:02, given the peptide sequence as an allele-interacting variable, and the N-terminal and C-terminal flanking sequences as allele-noninteracting variables.

As laid out in the following examples and with reference to FIGS. 13A-13E, various models, such as the trained presentation models and current state-of-the-art models for peptide binding prediction, are applied to the test set of neoantigen candidates for each simulated patient to identify different treatment subsets for patients based on the predictions. Patients that satisfy inclusion criteria are selected for vaccine treatment, and are associated with customized vaccines that include epitopes in the treatment subsets of the patients. The size of the treatment subsets are varied according to different vaccine capacities. No overlap is introduced between the training set used to train the presentation model and the test set of simulated neoantigen candidates.

In the following examples, the proportion of selected patients having at least a certain number of presented neoantigens among the epitopes included in the vaccines are analyzed. This statistic indicates the effectiveness of the simulated vaccines to deliver potential neoantigens that will elicit immune responses in patients. Specifically, a simulated neoantigen in a test set is presented if the neoantigen is presented in the mass spectrometry data set D2. A high proportion of patients with presented neoantigens indicate potential for successful treatment via neoantigen vaccines by inducing immune responses.

### XI.A. Example 7A: Frequency Distribution of Mutation Burden for NSCLC Cancer Patients

FIG. 13A illustrates a sample frequency distribution of mutation burden in NSCLC patients. Mutation burden and mutations in different tumor types, including NSCLC, can be found, for example, at the cancer genome atlas (TCGA) (https://cancersgenome.nih.gov). The x-axis represents the number of non-synonymous mutations in each patient, and the y-axis represents the proportion of sample patients that have the given number of non-synonymous mutations. The sample frequency distribution in FIG. 13A shows a range of 3-1786 mutations, in which 30% of the patients have fewer than 100 mutations. Although not shown in FIG. 13A, research indicates that mutation burden is higher in smokers compared to that of non-smokers, and that mutation burden may be a strong indicator of neoantigen load in patients.

As introduced at the beginning of Section XI above, each of a number of simulated patients are associated with a test set of neoantigen candidates. The test set for each patient is generated by sampling a mutation burden mᵢ from the frequency distribution shown in FIG. 13A for each patient. For each mutation, a 21-mer peptide sequence from the human proteome is randomly selected to represent a simulated mutated sequence. A test set of neoantigen candidate sequences are generated for patient i by identifying each (8, 9, 10, 11)-mer peptide sequence spanning the mutation in the 21-mer. Each neoantigen candidate is associated with a label indicating whether the neoantigen candidate sequence was present in the mass spectrometry D1 data set. For example, neoantigen candidate sequences present in data set D1 may be associated with a label “1,” while sequences not present in data set D1 may be associated with a label “0.” As described in more detail below, FIGS. 13B through 13E illustrate experimental results on patient selection based on presented neoantigens of the patients in the test set.

### XI.B. Example 7B: Proportion of Selected Patients with Neoantigen Presentation Based on Mutation Burden Inclusion Criteria

FIG. 13B illustrates the number of presented neoantigens in simulated vaccines for patients selected based on an inclusion criteria of whether the patients satisfy a minimum mutation burden. The proportion of selected patients that have at least a certain number of presented neoantigens in the corresponding test is identified.

In FIG. 13B, the x-axis indicates the proportion of patients excluded from vaccine treatment based on the minimum mutation burden, as indicated by the label “minimum # of mutations.” For example, a data point at 200 “minimum # of mutations” indicates that the patient selection module 324 selected only the subset of simulated patients having a mutation burden of at least 200 mutations. As another example, a data point at 300 “minimum # of mutations” indicates that the patient selection module 324 selected a lower proportion of simulated patients having at least 300 mutations. The y-axis indicates the proportion of selected patients that are associated with at least a certain number of presented neoantigens in the test set without any vaccine capacity v. Specifically, the top plot shows the proportion of selected patients that present at least 1 neoantigen, the middle plot shows the proportion of selected patients that present at least 2 neoantigens, and the bottom plot shows the proportion of selected patients that present at least 3 neoantigens.

As indicated in FIG. 13B, the proportion of selected patients with presented neoantigens increases significantly with higher mutation burden. This indicates that mutation burden as an inclusion criteria can be effective in selecting patients for whom neoantigen vaccines are more likely to induce successful immune responses.

### XI.C. Example 7C: Comparison of Neoantigen Presentation for Vaccines Identified by Presentation Models Vs. State-of-the-Art Models

FIG. 13C compares the number of presented neoantigens in simulated vaccines between selected patients associated with vaccines including treatment subsets identified based on presentation models and selected patients associated with vaccines including treatment subsets identified through current state-of-the-art models. The left plot assumes limited vaccine capacity v=10, and the right plot assumes limited vaccine capacity v=20. The patients are selected based on utility scores indicating expected number of presented neoantigens.

In FIG. 13C, the solid lines indicate patients associated with vaccines including treatment subsets identified based on presentation models for alleles HLA-A*02:01 and HLA-B*07:02. The treatment subset for each patient is identified by applying each of the presentation models to the sequences in the test set, and identifying the v neoantigen candidates that have the highest presentation likelihoods. The dotted lines indicate patients associated with vaccines including treatment subsets identified based on current state-of-the-art models NETMHCpan for the single allele HLA-A*02:01. Implementation details for NETMHCpan is provided in detail at http://www.cbs.dtu.dk/services/NetMHCpan. The treatment subset for each patient is identified by applying the NETMHCpan model to the sequences in the test set, and identifying the v neoantigen candidates that have the highest estimated binding affinities. The x-axis of both plots indicates the proportion of patients excluded from vaccine treatment based on expectation utility scores indicating the expected number of presented neoantigens in treatment subsets identified based on presentation models. The expectation utility score is determined as described in reference to equation (25) in Section X. The y-axis indicates the proportion of selected patients that present at least a certain number of neoantigens (1, 2, or 3 neoantigens) included in the vaccine.

As indicated in FIG. 13C, patients associated with vaccines including treatment subsets based on presentation models receive vaccines containing presented neoantigens at a significantly higher rate than patients associated with vaccines including treatment subsets based on state-of-the-art models. For example, as shown in the right plot, 80% of selected patients associated with vaccines based on presentation models receive at least one presented neoantigen in the vaccine, compared to only 40% of selected patients associated with vaccines based on current state-of-the-art models. The results indicate that presentation models as described herein are effective for selecting neoantigen candidates for vaccines that are likely to elicit immune responses for treating tumors.

### XI.D. Example 7D: Effect of HLA Coverage on Neoantigen Presentation for Vaccines Identified Through Presentation Models

FIG. 13D compares the number of presented neoantigens in simulated vaccines between selected patients associated with vaccines including treatment subsets identified based on a single per-allele presentation model for HLA-A*02:01 and selected patients associated with vaccines including treatment subsets identified based on both per-allele presentation models for HLA-A*02:01 and HLA-B*07:02. The vaccine capacity is set as v=20 epitopes. For each experiment, the patients are selected based on expectation utility scores determined based on the different treatment subsets.

In FIG. 13D, the solid lines indicate patients associated with vaccines including treatment subsets based on both presentation models for HLA alleles HLA-A*02:01 and HLA-B*07:02. The treatment subset for each patient is identified by applying each of the presentation models to the sequences in the test set, and identifying the v neoantigen candidates that have the highest presentation likelihoods. The dotted lines indicate patients associated with vaccines including treatment subsets based on a single presentation model for HLA allele HLA-A*02:01. The treatment subset for each patient is identified by applying the presentation model for only the single HLA allele to the sequences in the test set, and identifying the v neoantigen candidates that have the highest presentation likelihoods. For solid line plots, the x-axis indicates the proportion of patients excluded from vaccine treatment based on expectation utility scores for treatment subsets identified by both presentation models. For dotted line plots, the x-axis indicates the proportion of patients excluded from vaccine treatment based on expectation utility scores for treatment subsets identified by the single presentation model. The y-axis indicates the proportion of selected patients that present at least a certain number of neoantigens (1, 2, or 3 neoantigens).

As indicated in FIG. 13D, patients associated with vaccines including treatment subsets identified by presentation models for both HLA alleles present neoantigens at a significantly higher rate than patients associated with vaccines including treatment subsets identified by a single presentation model. The results indicate the importance of establishing presentation models with high HLA allele coverage.

### XI.E. Example 7E: Comparison of Neoantigen Presentation for Patients Selected by Mutation Burden Vs. Expected Number of Presented Neoantigens

FIG. 13E compares the number of presented neoantigens in simulated vaccines between patients selected based on mutation burden and patients selected by expectation utility score. The expectation utility scores are determined based on treatment subsets identified by presentation models having a size of v=20 epitopes.

In FIG. 13E, the solid lines indicate patients selected based on expectation utility score associated with vaccines including treatment subsets identified by presentation models. The treatment subset for each patient is identified by applying the presentation models to sequences in the test set, and identifying the v=20 neoantigen candidates that have the highest presentation likelihoods. The expectation utility score is determined based on the presentation likelihoods of the identified treatment subset based on equation (25) in section X. The dotted lines indicate patients selected based on mutation burden associated with vaccines also including treatment subsets identified by presentation models. The x-axis indicates the proportion of patients excluded from vaccine treatment based on expectation utility scores for solid line plots, and proportion of patients excluded based on mutation burden for dotted line plots. The y-axis indicates the proportion of selected patients who receive a vaccine containing at least a certain number of presented neoantigens (1, 2, or 3 neoantigens).

As indicated in FIG. 13E, patients selected based on expectation utility scores receive a vaccine containing presented neoantigens at a higher rate than patients selected based on mutation burden. However, patients selected based on mutation burden receive a vaccine containing presented neoantigens at a higher rate than unselected patients. Thus, mutation burden is an effective patient selection criteria for successful neoantigen vaccine treatment, though expectation utility scores are more effective.

### XII. Example 8: Evaluation of Mass Spectrometry-Trained MHC Class II Presentation Model on Held-Out MHC Class II Mass-Spectrometry Data

The validity of the various presentation models described above were tested on test data T that were subsets of training data 170 that were not used to train the presentation models or a separate dataset from the training data 170 that have similar variables and data structures as the training data 170.

A relevant metric indicative of the performance of a presentation models is:

$$Positive Predictive Value ( P P V ) = P ( {y}_{i ∈ T} = 1 \vert {u}_{i ∈ T} \geq t ) = \frac{{\sum }_{i ∈ T} ( {y}_{i} = 1 , {u}_{i} \geq t )}{{\sum }_{i ∈ T} ( {u}_{i} \geq t )}$$

that indicates the ratio of the number of peptide instances that were correctly predicted to be presented on associated HLA alleles to the number of peptide instances that were predicted to be presented on the HLA alleles. In one implementation, a peptide pⁱ in the test data T was predicted to be presented on one or more associated HLA alleles if the corresponding likelihood estimate uᵢ is greater or equal to a given threshold value t. Another relevant metric indicative of the performance of presentation models is:

$$Recall = P ( {u}_{i ∈ T} \geq t \vert {y}_{i ∈ T} = 1 ) = \frac{{\sum }_{i ∈ T} ( {y}_{i} = 1 , {u}_{i} \geq t )}{{\sum }_{i ∈ T} ( {y}_{i} = 1 )}$$

that indicates the ratio of the number of peptide instances that were correctly predicted to be presented on associated HLA alleles to the number of peptide instances that were known to be presented on the HLA alleles. Another relevant metric indicative of the performance of presentation models is the area-under-curve (AUC) of the receiver operating characteristic (ROC). The ROC lots the recall against the false positive rate FPR, which is given by:

$$FPR = P ( {u}_{i ∈ T} \geq t \vert {y}_{i ∈ T} = 0 ) = \frac{{\sum }_{i ∈ T} ( {y}_{i} = 0 , {u}_{i} \geq t )}{{\sum }_{i ∈ T} ( {y}_{i} = 0 )} .$$

### XII.A. Presentation Model Performance on MHC Class II Mass-Spectrometry Data

### XII.A.1. Example 1

FIG. 14A is a histogram of lengths of peptides eluted from class II MHC alleles on human tumor cells and tumor infiltrating lymphocytes (TIL) using mass spectrometry. Specifically, mass spectrometry peptidomics was performed on HLA-DRB1*12:01 homozygote alleles (“Dataset 1”) and HLA-DRB1*12:01, HLA-DRB1*10:01 multi-allele samples (“Dataset 2”). Results show that lengths of peptides eluted from class II MHC alleles range from 6-30 amino acids. The frequency distribution shown in FIG. 14A is similar to that of lengths of peptides eluted from class II MHC alleles using state-of-the-art mass spectrometry techniques, as shown in FIG. 1C of reference 69.

FIG. 14B illustrates the dependency between mRNA quantification and presented peptides per residue for Dataset 1 and Dataset 2. Results show that there is a strong dependency between mRNA expression and peptide presentation for class II MHC alleles.

Specifically, the horizontal axis in FIG. 14B indicates mRNA expression in terms of log₁₀ transcripts per million (TPM) bins. The vertical axis in FIG. 14B indicates peptide presentation per residue as a multiple of that of the lowest bin corresponding to mRNA expression between 10⁻²<log₁₀TPM<10⁻¹. One solid line is a plot relating mRNA quantification and peptide presentation for Dataset 1, and another is for Dataset 2. As shown in FIG. 14B, there is a strong positive correlation between mRNA expression, and peptide presentation per residue in the corresponding gene. Specifically, peptides from genes in the range of 10¹<log₁₀TPM<10² of RNA expression are more than 5 times likely to be presented than the bottom bin.

The results indicate that the performance of the presentation model can be greatly improved by incorporating mRNA quantification measurements, as these measurements are strongly predictive of peptide presentation.

FIG. 14C compares performance results for example presentation models trained and tested using Dataset 1 and Dataset 2. For each set of model features of the example presentation models, FIG. 14C depicts a PPV value at 10% recall when the features in the set of model features are classified as allele interacting features, and alternatively when the features in the set of model features are classified as allele non-interacting features variables. As seen in FIG. 14C, for each set of model features of the example presentation models, a PPV value at 10% recall that was identified when the features in the set of model features were classified as allele interacting features is shown on the left side, and a PPV value at 10% recall that was identified when the features in the set of model features were classified as allele non-interacting features is shown on the right side. Note that the feature of peptide sequence was always classified as an allele interacting feature for the purposes of FIG. 14C. Results showed that the presentation models achieved a PPV value at 10% recall varying from 14% up to 29%, which are significantly (approximately 500-fold) higher than PPV for a random prediction.

Peptide sequences of lengths 9-20 were considered for this experiment. The data was split into training, validation, and testing sets. Blocks of peptides of 50 residue blocks from both Dataset 1 and Dataset 2 were assigned to training and testing sets. Peptides that were duplicated anywhere in the proteome were removed, ensuring that no peptide sequence appeared both in the training and testing set. The prevalence of peptide presentation in the training and testing set was increased by 50 times by removing non-presented peptides. This is because Dataset 1 and Dataset 2 are from human tumor samples in which only a fraction of the cells are class II HLA alleles, resulting in peptide yields that were roughly 10 times lower than in pure samples of class II HLA alleles, which is still an underestimate due to imperfect mass spectrometry sensitivity. The training set contained 1,064 presented and 3,810,070 non-presented peptides. The test set contained 314 presented and 807,400 non-presented peptides.

Example model 1 was the sum-of-functions model in equation (22) using a network dependency function gₕ(⋅), the expit function f(⋅), and the identity function r(⋅). The network dependency function gₕ(⋅) was structured as a multi-layer perceptron (MLP) with 256 hidden nodes and rectified linear unit (ReLU) activations. In addition to the peptide sequence, the allele interacting variables w contained the one-hot encoded C-terminal and N-terminal flanking sequence, a categorical variable indicating index of source gene G=gene(pᵢ) of peptide pᵢ, and a variable indicating mRNA quantification measurement. Example model 2 was identical to example model 1, except that the C-terminal and N-terminal flanking sequence was omitted from the allele interacting variables. Example model 3 was identical to example model 1, except that the index of source gene was omitted from the allele interacting variables. Example model 4 was identical to example model 1, except that the mRNA quantification measurement was omitted from the allele interacting variables.

Example model 5 was the sum-of-functions model in equation (20) with a network dependency function gₕ(⋅), the expit function f(⋅),the identity function r(⋅), and the dependency function g_{w}(⋅) of equation (12). The dependency function g_{w}(⋅) also included a network model taking mRNA quantification measurement as input, structured as a MLP with 16 hidden nodes and ReLU activations, and a network model taking C-flanking sequence as input, structured as a MLP with 32 hidden nodes and ReLU activations. The network dependency function gₕ(⋅) was structured as a multi-layer perceptron with 256 hidden nodes and rectified linear unit (ReLU) activations. Example model 6 was identical to example model 5, except that the network model for C-terminal and N-terminal flanking sequence was omitted. Example model 7 was identical to example model 5, except that the index of source gene was omitted from the allele noninteracting variables. Example model 8 was identical to example model 5, except that the network model for mRNA quantification measurement was omitted.

The prevalence of presented peptides in the test set was approximately 1/2400, and therefore, the PPV of a random prediction would also be approximately 1/2400=0.00042. As shown in FIG. 14C, the best-performing presentation model achieved a PPV value of approximately 29%, which is roughly 500 times better than the PPV value of a random prediction.

### XII.A.2. Example 2

FIG. 14D is a histogram that depicts the quantity of peptides sequenced using mass spectrometry for each sample of a total of 73 samples comprising human tumors (NSCLC, lymphoma, and ovarian cancer) and cell lines (EBV) including HLA class II molecules. As shown in FIG. 14D, an average of 900 peptides were sequenced for each sample. Furthermore, for each sample of the plurality of samples, the histogram shown in FIG. 14D depicts the quantity of peptides sequenced using mass spectrometry at different q-value thresholds. Specifically, for each sample of the plurality of samples, FIG. 14D depicts the quantity of peptides sequenced using mass spectrometry with a q-value of less than 0.01, with a q-value of less than 0.05, and with a q-value of less than 0.2.

As noted above, each sample of the 73 samples of FIG. 14D comprised HLA class II molecules. More specifically, each sample of the 73 samples of FIG. 14D comprised HLA-DR molecules. The HLA-DR molecule is one type of HLA class II molecule. Even more specifically, each sample of the 73 samples of FIG. 14D comprised HLA-DRB1 molecules, HLA-DRB3 molecules, HLA-DRB4 molecules, and/or HLA-DRB5 molecules. The HLA-DRB1 molecule, the HLA-DRB3 molecule, the HLA-DRB4 molecule, and the HLA-DRB5 molecule are types of the HLA-DR molecule.

While this particular experiment was performed using samples comprising HLA-DR molecules, and particularly HLA-DRB1 molecules, HLA-DRB3 molecules, HLA-DRB4 molecules, and HLA-DRB5 molecules, in alternative embodiments, this experiment can be performed using samples comprising one or more of any type(s) of HLA class II molecules. For example, in alterative embodiments, identical experiments can be performed using samples comprising HLA-DP and/or HLA-DQ molecules. This ability to model any type(s) of MHC class II molecules using the same techniques, and still achieve reliable results, is well known by those skilled in the art. For instance, Jensen, Kamilla Kjaergaard, et al.⁷⁶ is one example of a recent scientific paper that uses identical methods for modeling binding affinity for HLA-DR molecules as well as for HLA-DQ and HLA-DP molecules. Therefore, one skilled in the art would understand that the experiments and models described herein can be used to separately or simultaneously model not only HLA-DR molecules, but any other MHC class II molecule, while still producing reliable results.

To sequence the peptides of each sample of the 73 total samples, mass spectrometry was performed for each sample. The resulting mass spectrum for the sample was then searched with Comet and scored with Percolator to sequence the peptides. Then, the quantity of peptides sequenced in the sample was identified for a plurality of different Percolator q-value thresholds. Specifically, for the sample, the quantity of peptides sequenced with a Percolator q-value of less than 0.01, with a Percolator q-value of less than 0.05, and with a Percolator q-value of less than 0.2 were determined.

For each sample of the 73 samples, the quantity of peptides sequenced at each of the different Percolator q-value thresholds is depicted in FIG. 14D. For example, as seen in FIG. 14D, for the first sample, approximately 4700 peptides with a q-value of less than 0.2 were sequenced using mass spectrometry, approximately 3600 peptides with a q-value of less than 0.05 were sequenced using mass spectrometry, and approximately 3200 peptides with a q-value of less than 0.01 were sequenced using mass spectrometry.

Overall, FIG. 14D demonstrates the ability to use mass spectrometry to sequence a large quantity of peptides from samples containing MHC class II molecules, at low q-values. In other words, the data depicted in FIG. 14D demonstrate the ability to reliably sequence peptides that may be presented by MHC class II molecules, using mass spectrometry.

FIG. 14E is a histogram that depicts the quantity of samples in which a particular MHC class II molecule allele was identified. More specifically, for the 73 total samples comprising HLA class II molecules, FIG. 14E depicts the quantity of samples in which certain MHC class II molecule alleles were identified.

As discussed above with regard to FIG. 14D, each sample of the 73 samples of FIG. 14D comprised HLA-DRB1 molecules, HLA-DRB3 molecules, HLA-DRB4 molecules, and/or HLA-DRB5 molecules. Therefore, FIG. 14E depicts the quantity of samples in which certain alleles for HLA-DRB1, HLA-DRB3, HLA-DRB4, and HLA-DRB5 molecules were identified. To identify the HLA alleles present in a sample, HLA class II DR typing is performed for the sample. Then, to identify the quantity of samples in which a particular HLA allele was identified, the number of samples in which the HLA allele was identified using HLA class II DR typing is simply summed. For example, as depicted in FIG. 14E, 17 samples of the 73 total samples contained the HLA class II molecule allele HLA-DRB3*01:01. In other words, 17 samples of the 73 total samples contained the allele HLA-DRB3*01:01 for the HLA-DRB3 molecule. Overall, FIG. 14E depicts the ability to identify a wide range of HLA class II molecule alleles from the 73 samples comprising HLA class II molecules.

FIG. 14F is a histogram that depicts the proportion of peptides presented by the MHC class II molecules in the 73 total samples, for each peptide length of a range of peptide lengths. To determine the length of each peptide in each sample of the 73 total samples, each peptide was sequenced using mass spectrometry as discussed above with regard to FIG. 14D, and then the number of residues in the sequenced peptide was simply quantified.

As noted above, MHC class II molecules typically present peptides with lengths of between 9-20 amino acids. Accordingly, FIG. 14F depicts the proportion of peptides presented by the MHC class II molecules in the 73 samples for each peptide length between 9-20 amino acids, inclusive. For example, as shown in FIG. 14F, approximately 23% of the peptides presented by the MHC class II molecules in the 73 samples comprise a length of 14 amino acids.

Based on the data depicted in FIG. 14F, modal lengths for the peptides presented by the MHC class II molecules in the 73 samples were identified to be 14 and 15 amino acids in length. These modal lengths identified for the peptides presented by the MHC class II molecules in the 73 samples are consistent with previous reports of modal lengths for peptides presented by MHC class II molecules. Additionally, as also consistent with previous reports, the data of FIG. 14F indicates that more than 60% of the peptides presented by the MHC class II molecules from the 73 samples comprise lengths other than 14 and 15 amino acids. In other words, FIG. 14F indicates that while peptides presented by MHC class II molecules are most frequently 14 or 15 amino acids in length, a large proportion of peptides presented by MHC class II molecules are not 14 or 15 amino acids in length. Accordingly, it is a poor assumption to assume that peptides of all lengths have equal probabilities of being presented by MHC class II molecules, or that only peptides that comprise a length of 14 or 15 amino acids are presented by MHC class II molecules. As discussed in detail below with regard to FIG. 14L, these faulty assumptions are currently used in many state-of-the-art models for predicting peptide presentation by MHC class II molecules, and therefore, the presentation likelihoods predicted by these models are often unreliable.

FIG. 14G is a line graph that depicts the relationship between gene expression and prevalence of presentation of the gene expression product by a MHC class II molecule, for genes present in the 73 samples. More specifically, FIG. 14G depicts the relationship between gene expression and the proportion of residues resulting from the gene expression that form the N-terminus of a peptide presented by a MHC class II molecule. To quantify gene expression in each sample of the 73 total samples, RNA sequencing is performed on the RNA included in each sample. In FIG. 14O, gene expression is measured by RNA sequencing in units of transcripts per million (TPM). To identify prevalence of presentation of gene expression products for each sample of the 73 samples, identification of HLA class II DR peptidomic data was performed for each sample.

As depicted in FIG. 14G, for the 73 samples, there is a strong correlation between gene expression level and presentation of residues of the expressed gene product by a MHC class II molecule. Specifically, as shown in FIG. 14G, peptides resulting from expression of the least-expressed genes are more than 100-fold less likely to be presented by a MHC class II molecule, than peptides resulting from expression of the most-expressed genes. In simpler terms, the products of more highly expressed genes are more frequently presented by MHC class II molecules.

FIGS. 14H-I and 14K-L are line graphs that compare the performance of various presentation models at predicting the likelihood that peptides in a testing dataset of peptides will be presented by at least one of the MHC class II molecules present in the testing dataset. As shown in FIGS. 14H-I and 14K-L, the performance of a model at predicting the likelihood that a peptide will be presented by at least one of the MHC class II molecules present in the testing dataset is determined by identifying a ratio of a true positive rate to a false positive rate for each prediction made by the model. These ratios identified for a given model can be visualized as a ROC (receiver operator characteristic) curve, in a line graph with an x-axis quantifying false positive rate and a y-axis quantifying true positive rate. An area under the curve (AUC) is used to quantify the performance of the model. Specifically, a model with a greater AUC has a higher performance (i.e., greater accuracy) relative to a model with a lesser AUC. In FIGS. 14H, 14I, and 14L, the blacked dashed line with a slope of 1 (i.e., a ratio of true positive rate to false positive rate of 1) depicts the expected curve for randomly guessing likelihoods of peptide presentation. The AUC for the dashed line is 0.5. ROC curves and the AUC metric are discussed in detail with regard to the top portion of Section XII, above.

FIG. 14H is a line graph that compares the performance of five example presentation models at predicting the likelihood that peptides in a testing dataset of peptides will be presented by a MHC class II molecule, given different sets of allele interacting and allele non-interacting variables. In other words, FIG. 14H quantifies the relative importance of various allele interacting and allele non-interacting variables for predicting the likelihood that a peptide will be presented by a MHC class II molecule.

The model architecture of each example presentation model of the five example presentations models used to generate the ROC curves of the line graph of FIG. 14H, comprised an ensemble of five sum-of-sigmoids models. Each sum-of-sigmoids model in the ensemble was configured to model peptide presentation for up to four unique HLA-DR alleles per sample. Furthermore, each sum-of-sigmoids model in the ensemble was configured to make predictions of peptide presentation likelihood based on the following allele interacting and allele non-interacting variables: peptide sequence, flanking sequence, RNA expression in units of TPM, gene identifier, and sample identifier. The allele interacting component of each sum-of-sigmoids model in the ensemble was a one-hidden-layer MLP with ReLu activations as 256 hidden units.

Prior to using the example models to predict the likelihood that the peptides in a testing dataset of peptides will be presented by a MHC class II molecule, the example models were trained and validated. To train, validate, and finally test the example models, the data described above for the 73 samples was split into training, validation, and testing datasets.

To ensure that no peptides appeared in more than one of the training, validation, and testing datasets, the following procedure was performed. First all peptides from the 73 total samples that appeared in more than one location in the proteome were removed. Then, the peptides from the 73 total samples were partitioned into blocks of 10 adjacent peptides. Each block of the peptides from the 73 total samples was assigned uniquely to the training dataset, the validation dataset, or the testing dataset. In this way, no peptide appeared in more than one dataset of the training, validation, and testing datasets.

Out of the 38,035,453 peptides in the 73 total samples, the training dataset comprised 33,570 peptides presented by MHC class II molecules from 69 of the 73 total samples. The 33,570 peptides included in the training dataset were between lengths of 9 and 20 amino acids, inclusive. The example models used to generate the ROC curves in FIG. 14H were trained on the training dataset using the ADAM optimizer and early stopping.

The validation dataset consisted of 3,925 peptides presented by MHC class II molecules from the same 69 samples used in the training dataset. The validation set was used only for early stopping.

The testing dataset comprised peptides presented by MHC class II molecules that were identified from a tumor sample using mass spectrometry. Specifically, the testing dataset comprised 232 peptides that were identified from four tumor sample. The peptides included in the testing dataset were held out of the training dataset described above.

As noted above, FIG. 14H quantifies the relative importance of various allele interacting variables and allele non-interacting variables for predicting the likelihood that a peptide will be presented by a MHC class II molecule. As also noted above, the example models used to generate the ROC curves of the line graph of FIG. 14H were configured to make predictions of peptide presentation likelihood based on the following allele interacting and allele non-interacting variables: peptide sequence, flanking sequence, RNA expression in units of TPM, gene identifier, and sample identifier. To quantify the relative importance of four of these five variables (peptide sequence, flanking sequence, RNA expression, and gene identifier) for predicting the likelihood that a peptide will be presented by a MHC class II molecule, each example model of the five the example models described above was tested using data from the testing dataset, with a different combination of the four variables. Specifically, for each peptide of the testing dataset, an example model 1 generated predictions of peptide presentation likelihood based on a peptide sequence, a flanking sequence, a gene identifier, and a sample identifier, but not on RNA expression. Similarly, for each peptide of the testing dataset, an example model 2 generated predictions of peptide presentation likelihood based on a peptide sequence, RNA expression, a gene identifier, and a sample identifier, but not on a flanking sequence. Similarly, for each peptide of the testing dataset, an example model 3 generated predictions of peptide presentation likelihood based on a flanking sequence, RNA expression, a gene identifier, and a sample identifier, but not on a peptide sequence. Similarly, for each peptide of the testing dataset, an example model 4 generated predictions of peptide presentation likelihood based on a flanking sequence, RNA expression, a peptide sequence, and a sample identifier, but not on a gene identifier. Finally, for each peptide of the testing dataset, an example model 5 generated predictions of peptide presentation likelihood based on all five variables of flanking sequence, RNA expression, peptide sequence, sample identifier, and gene identifier.

The performance of each of these five example models is depicted in the line graph of FIG. 14H. Specifically, each of the five example models is associated with a ROC curve that depicts a ratio of a true positive rate to a false positive rate for each prediction made by the model. For instance, FIG. 14H depicts a curve for the example model 1 that generated predictions of peptide presentation likelihood based on a peptide sequence, a flanking sequence, a gene identifier, and a sample identifier, but not on RNA expression. FIG. 14H depicts a curve for the example model 2 that generated predictions of peptide presentation likelihood based on a peptide sequence, RNA expression, a gene identifier, and a sample identifier, but not on a flanking sequence. FIG. 14H also depicts a curve for the example model 3 that generated predictions of peptide presentation likelihood based on a flanking sequence, RNA expression, a gene identifier, and a sample identifier, but not on a peptide sequence. FIG. 14H also depicts a curve for the example model 4 that generated predictions of peptide presentation likelihood based on a flanking sequence, RNA expression, a peptide sequence, and a sample identifier, but not on a gene identifier. And finally FIG. 14H depicts a curve for the example model 5 that generated predictions of peptide presentation likelihood based on all five variables of flanking sequence, RNA expression, peptide sequence, sample identifier, and gene identifier.

As noted above, the performance of a model at predicting the likelihood that a peptide will be presented by a MHC class II molecule is quantified by identifying an AUC for a ROC curve that depicts a ratio of a true positive rate to a false positive rate for each prediction made by the model. A model with a greater AUC has a higher performance (i.e., greater accuracy) relative to a model with a lesser AUC. As shown in FIG. 14H, the curve for the example model 5 that generated predictions of peptide presentation likelihood based on all five variables of flanking sequence, RNA expression, peptide sequence, sample identifier, and gene identifier, achieved the highest AUC of 0.98. Therefore the example model 5 that used all five variables to generate predictions of peptide presentation achieved the best performance. The curve for the example model 2 that generated predictions of peptide presentation likelihood based on a peptide sequence, RNA expression, a gene identifier, and a sample identifier, but not on a flanking sequence, achieved the second highest AUC of 0.97. Therefore, the flanking sequence can be identified as the least important variable for predicting the likelihood that a peptide will be presented by a MHC class II molecule. The curve for the example model 4 generated predictions of peptide presentation likelihood based on a flanking sequence. RNA expression, a peptide sequence, and a sample identifier, but not on a gene identifier, achieved the third highest AUC of 0.96. Therefore, the gene identifier can be identified as the second least important variable for predicting the likelihood that a peptide will be presented by a MHC class II molecule. The curve for the example model 3 that generated predictions of peptide presentation likelihood based on a flanking sequence, RNA expression, a gene identifier, and a sample identifier, but not on a peptide sequence, achieved the lowest AUC of 0.88. Therefore, the peptide sequence can be identified as the most important variable for predicting the likelihood that a peptide will be presented by a MHC class II molecule. The curve for the example model 1 that generated predictions of peptide presentation likelihood based on a peptide sequence, a flanking sequence, a gene identifier, and a sample identifier, but not on RNA expression, achieved the second lowest AUC of 0.95. Therefore, RNA expression can be identified as the second most important variable for predicting the likelihood that a peptide will be presented by a MHC class II molecule.

FIG. 14I is a line graph that compares the performance of four different presentation models at predicting the likelihood that peptides in a testing dataset of peptides will be presented by a MHC class II molecule.

The first model tested in FIG. 14I is referred to herein as a “Binding Affinity” model. The Binding Affinity model of FIG. 14I is a best-in-class prior art model, the NetMHCII 2.3 model, that utilizes minimum NetMHCII 2.3 predicted binding affinity as a criterion to generate predictions. Specifically, the NetMHCII 2.3 model generates predictions of peptide presentation likelihood based on MHC class II molecule type and peptide sequence. The NetMHCII 2.3 model was tested using the NetMHCII 2.3 website (www.cbs.dtu.dk/services/NetMHCII/, PMID 29315598)⁷⁶.

The second model tested in FIG. 14I is referred to herein as an “MLP” model. The MLP (multi-layer perceptron) model is one embodiment of the presentation models described above in which allele-noninteracting variables wₖ and allele-interacting variables xₕ^{k} are input into separate dependency functions such as, for example, a neural network, and then the outputs of these separate dependency functions are added. Specifically, the full non-interacting model is one embodiment of the presentation models described above in which allele-noninteracting variables w^{k} are input into a dependency function g, allele-interacting variables xₕ^{k} are input into separate dependency function gₕ, and the outputs of the dependency function g_{w} and the dependency function gₕ are added together. Therefore, in some embodiments, the full non-interacting model determines the likelihood of peptide presentation using equation 8 as shown above. Furthermore, embodiments of the full non-interacting model in which allele-noninteracting variables w^{k} are input into a dependency function gₕ, allele-interacting variables xₕ^{k} are input into separate dependency function gₕ, and the outputs of the dependency function g_{w} and the dependency function gₕ are added, are discussed in detail above with regard to the top portion of Section VIII.B.2., the bottom portion of Section VIII.B.3., the top portion of Section VIII.C.3., and the top portion of Section VIII.C.6.

The third model tested in FIG. 14I is referred to herein as a “RNN” model. The RNN model comprises a recurrent neural network, and is similar to the full non-interacting model described above. However, the layers of the recurrent neural network of the RNN model differ from the layers of the neural network of the MLP model. Specifically, the input layer of the recurrent neural network of the RNN model accepts a variable length peptide string that is modeled one peptide at a time. The peptide is fed a single amino acid at a time into a neural network node whose output is piped into the node's input along with the next amino acid in the sequence until the entire sequence has been modeled. A recurrent layer is especially applicable to MHC class II peptide modeling for two reasons: (1) the sequential nature of the data is captured by the model and (2) the peptides can vary in length without the need for artificially padding. The next layers of the recurrent neural network is a dropout layer with p=0.2, and finally a dense 64-node layer with a ReLu activation.

The fourth model tested in FIG. 14I is referred to herein as a “Bi-LSTM” model. The Bi-LSTM model comprises a bi-directional long short-term memory neural network. The Bi-LSTM model is identical to the non-interacting model except for the peptide input layer. The input layer of the Bi-LSTM model accepts a 20-mer peptide string and subsequently embeds the 20-mer peptide string as a (n, 20, 21) tensor. The next layers of the bi-directional long short-term memory neural network of the Bi-LSTM model comprise a recurrent long short-term memory layer with 128 nodes, a dropout layer with p=0.2, and finally a dense 64-node layer with a ReLu activation. In traditional LSTM models, the order of the sequential data is assumed to be directional (e.g., read left to right or right to left). In a bidirectional LSTM, the sequential data are processed in both directions, going left to right and right to left. Peptide binding is an inherently directionless task, and so modeling the sequence in both direction ensures information from either end of the sequence will hold as much weight in the model's prediction.

Turning briefly to FIG. 14J, FIG. 14J depicts an exemplar embodiment of the Bi-LSTM model of FIG. 14I, configured to predict peptide presentation by HLA-DRB (a MHC class II gene). As shown in FIG. 14J, the Bi-LSTM model comprises a shared neural network that accepts allele non-interacting features (e.g., RNA sequences, sample IDs, protein IDs, and flanking sequences) and a set of distinct neural networks, each associated with a different HLA-DRB allele and configured to accept an encoded peptide sequence (an allele interacting feature). Each distinct neural network of the set of neural networks comprises a Bi-LSTM neural network. In the exemplar embodiment of the Bi-LSTM model of FIG. 14J, the set of distinct neural networks associated with different alleles comprises 4 distinct neural networks because the HLA-DRB gene is associated with at most 4 different alleles per patient sample. However, in alternative embodiments in which the Bi-LSTM model is configured to predict peptide presentation by another HLA gene, the set of distinct neural networks comprises a quantity of distinct neural networks equal to the maximum possible quantity of alleles in a patient sample for the given HLA gene. Each distinct neural network of the set of neural networks determines a likelihood that the peptide input into the model will be presented by the HLA-DBR allele associated with the given neural network. Each of these likelihoods is then combined with the output from the shared neural network. Finally, the combined likelihoods are summed to generate an overall likelihood that the peptide will be presented by the HLA-DBR gene.

Turning back to FIG. 14I, prior to using each of the four models of FIG. 14I to predict the likelihood that the peptides in the testing dataset of peptides will be presented by a MHC class II molecule, the models were trained and validated. The Binding Affinity model was trained and validated using its own training and validation datasets based on HLA-peptide binding affinity assays deposited in the immune epitope database (IEDB, www.iedb.org). The other three models were trained using the 69-sample training dataset described above and validated using the validation dataset described above. Following this training and validation of the models, each of the four models was tested using 4 held-out tumor samples from the testing dataset described above. Specifically, for each of the four models, each peptide of the 4 held-out tumor samples from the testing dataset was input into the model, and the model subsequently output a presentation likelihood for the peptide.

The performance of each of the four models is depicted in the line graph in FIG. 14I. Specifically, each of the four models is associated with a ROC curve that depicts a ratio of a true positive rate to a false positive rate for each prediction made by the model. For instance, FIG. 14I depicts a ROC curve for the Binding Affinity model, a ROC curve for the RNN model, a ROC curve for the MLP model, and a ROC curve for the Bi-LSTM model.

As noted above, the performance of a model at predicting the likelihood that a peptide will be presented by a MHC class II molecule is quantified by identifying an AUC for a ROC curve that depicts a ratio of a true positive rate to a false positive rate for each prediction made by the model. A model with a greater AUC has a higher performance (i.e., greater accuracy) relative to a model with a lesser AUC. As shown in FIG. 14I, the curve for the Bi-LSTM model achieved the highest AUC of 0.98. Therefore the Bi-LSTM model achieved the best performance. This peak performance of the Bi-LSTM model is due in part to the fact that the Bi-LSTM has the greatest ability to accurately predict peptides of variable length, peptides of relatively longer length, and peptides with repeating amino acids. The curves for the MLP and RNN models achieved the second highest AUCs of 0.97. Therefore, the MLP and RNN models achieved the second best performance. The curve for the Binding Affinity model achieved the lowest AUC of 0.79. Therefore the Binding Affinity model achieved the worst performance. Note that each of the Bi-LSTM, MLP, and RNN models tested in FIG. 14I has an AUC that is greater than 0.9. Accordingly, despite the architectural variance in the peptide input layer between them, these models are capable of achieving relatively accurate predictions of peptide presentation, unlike the Binding Affinity model which has a much lower AUC.

FIG. 14K is a line graph that depicts full precision-recall curves for the “Bi-LSTM” model, the “MLP” model, the “RNN” model, and the “Binding Affinity” model discussed above with regard to FIG. 14I. As shown in FIG. 14K, and as expected based on FIG. 14I, the “Bi-LSTM” model achieved the best performance with an AUC of 0.23, the “RNN” model achieved the second best performance with an AUC of 0.16, the “MLP” model achieved the third best performance with an AUC of 0.11, and the “Binding Affinity” model achieved the worst performance with an AUC of 0.01. In particular, the Bi-LSTM model trained with mass spectrometry data significantly outperforms the Binding Affinity model, with a greater than 20-fold increase in AUC.

FIG. 14L is a line graph that compares the performance of two example best-in-class prior art models given two different criteria, and two example presentation models given two different sets of allele interacting and allele non-interacting variables, at predicting the likelihood that peptides in a testing dataset of peptides will be presented by a MHC class II molecule. Specifically, FIG. 14L is a line graph that compares the performance of an example best-in-class prior art model that utilizes minimum NetMHCII 2.3 predicted binding affinity as a criterion to generate predictions (example model 1), an example best-in-class prior art model that utilizes minimum NetMHCII 2.3 predicted binding rank as a criterion to generate predictions (example model 2), an example presentation model that generates predictions of peptide presentation likelihood based on MHC class II molecule type and peptide sequence (example model 4), and an example presentation model that generates predictions of peptide presentation likelihood based on MHC class II molecule type, peptide sequence, RNA expression, gene identifier, and flanking sequence (example model 3).

The best-in-class prior art model used as example model 1 and example model 2 in FIG. 14L is the NetMHCII 2.3 model. The NetMHCII 2.3 model generates predictions of peptide presentation likelihood based on MHC class II molecule type and peptide sequence. The NetMHCII 2.3 model was tested using the NetMHCII 2.3 website (www.cbs.dtu.dk/services/NetMHCHII/, PMID 29315598)⁷⁶.

As noted above, the NetMHCII 2.3 model was tested according to two different criteria. Specifically, example model 1 model generated predictions of peptide presentation likelihood according to minimum NetMHCII 2.3 predicted binding affinity, and example model 2 generated predictions of peptide presentation likelihood according to minimum NetMHCII 2.3 predicted binding rank.

The presentation model used as example model 3 and example model 4 is an embodiment of the presentation model disclosed herein that is trained using data obtained via mass spectrometry. As noted above, the presentation model generated predictions of peptide presentation likelihood based on two different sets of allele interacting and allele non-interacting variables. Specifically, example model 4 generated predictions of peptide presentation likelihood based on MHC class II molecule type and peptide sequence (the same variable used by the NetMHCII 2.3 model), and example model 3 generated predictions of peptide presentation likelihood based on MHC class II molecule type, peptide sequence, RNA expression, gene identifier, and flanking sequence.

Prior using the example models of FIG. 14L to predict the likelihood that the peptides in the testing dataset of peptides will be presented by a MHC class II molecule, the models were trained and validated. The NetMHCII 2.3 model (example model 1 and example model 2) was trained and validated using its own training and validation datasets based on HLA-peptide binding affinity assays deposited in the immune epitope database (IEDB, www.iedb.org). The training dataset used to train the NetMHCII 2.3 model is known to comprise almost exclusively 15-mer peptides. On the other hand, example models 3 and 4 were trained using the training dataset described above with regard to FIG. 14H and validated and using the validation dataset described above with regard to FIG. 14H.

Following the training and validation of the models, each of the models was tested using a testing dataset. As noted above, the NetMHCII 2.3 model is trained on a dataset comprising almost exclusively 15-mer peptides, meaning that NetMHCII 2.3 does not have the ability to give different priority to peptides of different weights, thereby reducing the predictive performance for NetMHCII 2.3 on HLA class II presentation mass spectrometry data containing peptides of all lengths. Therefore, to provide a fair comparison between the models not affected by variable peptide length, the testing dataset included exclusively 15-mer peptides. Specifically, the testing dataset comprised 933 15-mer peptides. 40 of the 933 peptides in the testing dataset were presented by MHC class II molecules-specifically by HLA-DRB1*07:01, HLA-DRB1*15:01, HLA-DRB4*01:03, and HLA-DRB5*01:01 molecules. The peptides included in the testing dataset were held out of the training datasets described above.

To test the example models using the testing dataset, for each of the example models, for each peptide of the 933 peptides in the testing dataset, the model generated a prediction of presentation likelihood for the peptide. Specifically, for each peptide in the testing dataset, the example 1 model generated a presentation score for the peptide by the MHC class II molecules using MHC class II molecule types and peptide sequence, by ranking the peptide by the minimum NetMHCII 2.3 predicted binding affinity across the four HLA class II DR alleles in the testing dataset. Similarly, for each peptide in the testing dataset, the example 2 model generated a presentation score for the peptide by the MHC class II molecules using MHC class II molecule types and peptide sequence, by ranking the peptide by the minimum NetMHCII 2.3 predicted binding rank (i.e., quantile normalized binding affinity) across the four HLA class II DR alleles in the testing dataset. For each peptide in the testing dataset, the example 4 model generated a presentation likelihood for the peptide by the MHC class II molecules based on MHC class II molecule type and peptide sequence. Similarly, for each peptide in the testing dataset, the example model 3 generated a presentation likelihood for the peptide by the MHC class II molecules based on MHC class II molecule types, peptide sequence, RNA expression, gene identifier, and flanking sequence.

The performance of each of the four example models is depicted in the line graph in FIG. 14L. Specifically, each of the four example models is associated with a ROC curve that depicts a ratio of a true positive rate to a false positive rate for each prediction made by the model. For instance, FIG. 14L depicts a ROC curve for the example 1 model that utilized minimum NetMHCII 2.3 predicted binding affinity to generate predictions, a ROC curve for the example 2 model that utilized minimum NetMHCII 2.3 predicted binding rank to generate predictions, a ROC curve for the example 4 model that generated peptide presentation likelihoods based on MHC class II molecule type and peptide sequence, and a ROC curve for the example 3 model that generated peptide presentation likelihoods based on MHC class II molecule type, peptide sequence, RNA expression, gene identifier, and flanking sequence.

As noted above, the performance of a model at predicting the likelihood that a peptide will be presented by a MHC class II molecule is quantified by identifying an AUC for a ROC curve that depicts a ratio of a true positive rate to a false positive rate for each prediction made by the model. A model with a greater AUC has a higher performance (i.e., greater accuracy) relative to a model with a lesser AUC. As shown in FIG. 14L, the curve for the example 3 model that generated peptide presentation likelihoods based on MHC class II molecule type, peptide sequence, RNA expression, gene identifier, and flanking sequence, achieved the highest AUC of 0.95. Therefore the example 3 model that generated peptide presentation likelihoods based on MHC class II molecule type, peptide sequence, RNA expression, gene identifier, and flanking sequence achieved the best performance. The curve for the example 4 model that generated peptide presentation likelihoods based on MHC class II molecule type and peptide sequence achieved the second highest AUC of 0.91. Therefore, the example 4 model that generated peptide presentation likelihoods based on MHC class II molecule type and peptide sequence achieved the second best performance. The curve for the example 1 model that utilized minimum NetMHCII 2.3 predicted binding affinity to generate predictions achieved the lowest AUC of 0.75. Therefore the example I model that utilized minimum NetMHCII 2.3 predicted binding affinity to generate predictions achieved the worst performance. The curve for the example 2 model that utilized minimum NetMHCII 2.3 predicted binding rank to generate predictions achieved the second lowest AUC of 0.76. Therefore, the example 2 model that utilized minimum NetMHCII 2.3 predicted binding rank to generate predictions achieved the second worst performance.

As shown in FIG. 14L, the discrepancy in performance between the example models 1 and 2 and the example models 3 and 4 is large. Specifically, the performance of the NetMHCII 2.3 model (that utilizes either criterion of minimum NetMHCII 2.3 predicted binding affinity or minimum NetMHCII 2.3 predicted binding rank) is almost 25% lower than the performance of the presentation model disclosed herein (that generates peptide presentation likelihoods based on either MHC class II molecule type and peptide sequence, or on MHC class II molecule type, peptide sequence, RNA expression, gene identifier, and flanking sequence). Therefore, FIG. 14L demonstrates that the presentation models disclosed herein are capable of achieving significantly more accurate presentation predictions than the current best-in-class prior art model, the NetMHCII 2.3 model.

Even further, as discussed above, the NetMHCII 2.3 model is trained on a training dataset that comprises almost exclusively 15-mer peptides. As a result, the NetMHCII 2.3 model is not trained to learn which peptides lengths are more likely to be presented by MHC class II molecules. Therefore, the NetMHCII 2.3 model does not weight its predictions of likelihood of peptide presentation by MHC class II molecules according to the length of the peptide. In other words, the NetMHCII 2.3 model does not modify its predictions of likelihood of peptide presentation by MHC class II molecules for peptides that have lengths outside of the modal peptide length of 15 amino acids. As a result, the NetMHCII 2.3 model overpredicts the likelihood of presentation of peptides with lengths greater or less than 15 amino acids.

On the other hand, the presentation models disclosed herein are trained using peptide data obtained via mass spectrometry, and therefore can be trained on training dataset that comprise peptides of all different lengths. As a result, the presentation models disclosed herein are able to learn which peptides lengths are more likely to be presented by MHC class II molecules. Therefore, the presentation models disclosed herein can weight predictions of likelihood of peptide presentation by MHC class II molecules according to the length of the peptide. In other words, the presentation models disclosed herein are able to modify their predictions of likelihood of peptide presentation by MHC class II molecules for peptides that have lengths outside of the modal peptide length of 15 amino acids. As a result, the presentation models disclosed herein are capable of achieving significantly more accurate presentation predictions for peptides of lengths greater than or less than 15 amino acids, than the current best-in-class prior art model, the NetMHCII 2.3 model. This is one advantage of using the presentation models disclosed herein to predict likelihood of peptide presentation by MHC class II molecules.

### XII.A.3. Example 3

FIG. 14M is a histogram that depicts the quantity of peptides sequenced using mass spectrometry at a q-value of less than 0.1 for each sample of a total of 230 samples comprising human tumors (NSCLC, lymphoma, and ovarian cancer) and cell lines (EBV) including HLA class II molecules. As shown in FIG. 14M, an average of 1300 peptides were sequenced for each sample at a q-value of less than 0.1.

As described above with regard to FIG. 14D, each sample of the 230 samples of FIG. 14M comprised HLA class II molecules. More specifically, each sample of the 230 samples of FIG. 14M comprised HLA-DR molecules. The HLA-DR molecule is one type of HLA class II molecule. Even more specifically, each sample of the 230 samples of FIG. 14M comprised HLA-DRB1 molecules, HLA-DRB3 molecules, HLA-DRB4 molecules, and/or HLA-DRB5 molecules. The HLA-DRB1 molecule, the HLA-DRB3 molecule, the HLA-DRB4 molecule, and the HLA-DRB5 molecule are types of the HLA-DR molecule.

While this particular experiment was performed using samples comprising HLA-DR molecules, and particularly HLA-DRB1 molecules, HLA-DRB3 molecules, HLA-DRB4 molecules, and HLA-DRB5 molecules, in alternative embodiments, this experiment can be performed using samples comprising one or more of any type(s) of HLA class II molecules. For example, in alterative embodiments, identical experiments can be performed using samples comprising HLA-DP and/or HLA-DQ molecules. This ability to model any type(s) of MHC class II molecules using the same techniques, and still achieve reliable results, is well known by those skilled in the art. For instance, Jensen. Kamilla Kjaergaard, et al.⁷⁶ is one example of a recent scientific paper that uses identical methods for modeling binding affinity for HLA-DR molecules as well as for HLA-DQ and HLA-DP molecules. Therefore, one skilled in the art would understand that the experiments and models described herein can be used to separately or simultaneously model not only HLA-DR molecules, but any other MHC class II molecule, while still producing reliable results.

To sequence the peptides of each sample of the 230 total samples, mass spectrometry was performed for each sample. The resulting mass spectrum for the sample was then searched with Comet and scored with Percolator to sequence the peptides. Then, the quantity of peptides sequenced in the sample was identified for a plurality of different Percolator q-value thresholds. Specifically, for the sample, the quantity of peptides sequenced with a Percolator q-value of less than 0.01, with a Percolator q-value of less than 0.05, and with a Percolator q-value of less than 0.2 were determined.

For each sample of the 203 samples, the quantity of peptides sequenced at each of the different Percolator q-value thresholds is depicted in FIG. 14M. For example, as seen in FIG. 14M, for the first sample, approximately 8000 peptides with a q-value of less than 0.1 were sequenced using mass spectrometry.

Overall, FIG. 14M demonstrates the ability to use mass spectrometry to sequence a large quantity of peptides from samples containing MHC class II molecules, at low q-values. In other words, the data depicted in FIG. 14M demonstrate the ability to reliably sequence peptides that may be presented by MHC class II molecules, using mass spectrometry.

FIG. 14N is a histogram that depicts the quantity of samples in which a particular MHC class II molecule allele was identified. More specifically, for the 230 total samples comprising HLA class II molecules, FIG. 14N depicts the quantity of samples in which certain MHC class II molecule alleles were identified.

As discussed above with regard to FIG. 14M, each sample of the 230 samples of FIG. 14M comprised HLA-DRB1 molecules. HLA-DRB3 molecules, HLA-DRB4 molecules, and/or HLA-DRB5 molecules. Therefore, FIG. 14N depicts the quantity of samples in which certain alleles for HLA-DRB1, HLA-DRB3, HLA-DRB4, and HLA-DRB5 molecules were identified.

To identify which HLA-DRB1, HLA-DRB3, HLA-DRB4, and HLA-DRB5 alleles were present in a sample, HLA class II DR typing was performed for the sample. Then, to identify the quantity of samples in which a particular HLA allele was identified, the number of samples in which the HLA allele was identified using HLA class II DR typing was simply summed. For example, as depicted in FIG. 14N, 28 samples of the 230 total samples contained the HLA class I molecule allele HLA-DRB3*03:01. In other words, 28 samples of the 230 total samples contained the allele HLA-DRB3*03:01 for the HLA-DRB3 molecule. Overall, FIG. 14N depicts the ability to identify a wide range of HLA class II molecule alleles from the 230 samples comprising HLA class II molecules. For reference to the human population, the allele frequencies of the HLA-DRB1 alleles in the Caucasian population of the United States can be found at Maiers, M, et al.¹⁶¹

FIG. 14O depicts a peptide bound to a MHC class I molecule and peptide bound to a MHC class II molecule.¹⁶² As shown in FIG. 14O, each peptide comprises a peptide backbone and a plurality of amino acids. Each MHC molecule comprises a binding grove. However, as discussed below, peptides bind differently within the binding groves of MHC class I and MHC class II molecules.

As discussed throughout this disclosure, peptides that are presented by MHC molecules can vary in length. Specifically, peptides that are presented by MHC molecules can be between 9-20 amino acids in length. When a peptide is bound to and presented by a MHC molecule, a “binding core” of the peptide is located within the binding groove of the MHC molecule. Specifically, a binding core of a peptide is the sequence of amino acids of the peptide that is located within a binding groove of a MHC molecule when the peptide is bound to and presented by the MHC molecule. Furthermore, when a peptide is bound to and presented by a MHC molecule, “binding anchors” of the binding core of the peptide physically bind to the binding groove of the MHC molecule. Specifically, a binding anchor of a binding core of a peptide is a specific amino acid of the binding core that binds to a binding groove of a MHC molecule when the peptide is bound to and presented by the MHC molecule.

As shown in FIG. 14O, the binding core of a peptide presented by a MHC class I molecule comprises the entire length of the peptide. Specifically, as shown in FIG. 14O, the entire peptide presented by the MHC class I molecule is located within the binding groove of the MHC class I molecule. In contrast, for a peptide presented by an MHC class II molecule, only a sub-sequence of amino acids of the peptide may be included in the binding core of the peptide. Specifically, as shown in FIG. 14O, the ends of the peptide presented by the MHC class II molecule are not located within the binding groove of the MHC class II molecule. The sub-sequence of amino acids comprising the binding core of a peptide presented by an MHC class II molecule may be unknown. However, as acknowledged in the literature, the most common length of a binding core of an MHC class II-presented peptide is 9 amino acids.

Furthermore, in addition to the binding core of a MHC class II-presented peptide being unknown, the quantity and positions of amino acids comprising the binding anchors of the binding core of the peptide may also be known. However, as acknowledged in the literature, a binding core of an MHC class II-presented peptide typically includes 34 binding anchors, and binding anchors typically include the amino acids located at the ends of the binding core.

Because of the distinction between peptide binding to MHC class I and MHC class II molecules, to ensure optimal peptide presentation prediction performance, peptide presentation prediction models should be configured to specifically predict MHC class II molecule peptide presentation. Specifically, because the sub-sequence of amino acids comprising a binding core and the binding anchors of the binding core of a peptide presented by an MHC class II molecule may be unknown, MHC class II peptide presentation prediction models should be configured to model this uncertainty. In particular, the Inception model was developed to model the uncertainty in binding core and binding anchor locations for peptides presented by MHC class II molecules.

FIG. 14P depicts an exemplar embodiment of an Inception neural network of the Inception model of FIG. 14Q, configured to predict peptide presentation by MHC class II molecules. The Inception model is a presentation model designed to identify the binding core and binding anchors of a peptide presented by MHC class II molecules, and to use these identified binding core and binding anchors to predict peptide presentation by MHC class II molecules. The Inception model comprises a shared neural network that accepts allele non-interacting features (e.g., RNA sequences, sample IDs, protein IDs, and flanking sequences) and a set of distinct Inception neural networks that accepts allele interacting features (e.g., peptide sequences). Specifically, each distinct Inception neural network in the set of distinct Inception neural networks is associated with a different MHC class II allele (e.g., a HLA-DRB allele), and is configured to accept an encoded peptide sequence. As mentioned above, FIG. 14P depicts an exemplar embodiment of an Inception neural network of an Inception model.

First, because peptides presented by MHC class II molecules are variable in length (e.g., between 9-20 amino acids), peptides that are shorter than the maximum length of 20 amino acids are padded to have a length of 20 amino acids. Specifically, if the length of a peptide is less than 20 amino acids, a special amino acid Z is added to the left of the peptide and then to the right of the peptide. This pattern of padding the peptide is repeated until the peptide has the length of 20 amino acids. By padding to the sides of the peptide, the binding core of the peptide is kept intact while peptide length is made constant across all peptides.

The input layer of an Inception neural network accepts a padded peptide sequence. The padded peptide is then one-hot encoded. As depicted in FIG. 14P, each Inception neural network includes three one-dimensional CNN layers. One of the three CNN layers has 16 filters of size 8. One of the three CNN layers has 16 filters of size 10. One of the three CNN layers has 16 filters of size 12. These filter sizes were intentionally selected to focus the Inception neural network on identifying a binding core of about 9 amino acids, which, as mentioned above, has been indicated in the literature as the most common binding core length for MHC class II-presented peptides.

The output of each of the three CNN layers is input into two one-dimensional CNN layers. One of the two CNN layers has 32 filters of size 1. One of the two CNN layers has 32 filters of size 2. These filter sizes were intentionally selected to identify the positions of binding anchors within the binding core of the MHC class II-presented peptide.

The outputs of these two CNN layers are concatenated. Each concatenated output is then fed to a bi-LSTM layer. The outputs of the bi-LSTM layers are concatenated, and this concatenation is sent to multi-layer perceptron. The output of the multi-layer perceptron comprises the output of the distinct Inception neural network. In other words, the output of the multi-layer perceptron comprises the likelihood that the peptide input into the distinct Inception neural network will be presented by the MHC class II allele associated with the distinct Inception neural network. The presentation likelihood from each distinct Inception neural network is combined with the output from the shared neural network. Finally, the combined likelihoods are summed to generate an overall likelihood that the peptide will be presented by one or more of the MHC class II alleles.

FIG. 14Q is a line graph that compares the performance of the “Bi-LSTM” and the “Inception” presentation models at predicting the likelihood that peptides in a testing dataset of peptides will be presented by at least one of the MHC class II molecules present in the testing dataset. Specifically, FIG. 14Q is a line graph that depicts full precision-recall curves for the “Bi-LSTM” model and the “Inception” model. AUC is used to quantify the performance of each model.

The first model tested in FIG. 14Q is the “Bi-LSTM” model. The Bi-LSTM model is the model discussed in detail above with regard to FIGS. 14I and 14J.

The second model tested in FIG. 14Q is the “Inception” model. The Inception model is the model discussed in detail above with regard to FIG. 14P.

Prior to using the models to predict the likelihood that the peptides in a testing dataset of peptides will be presented by a MHC class II molecule, the example models were trained and validated. To train, validate, and finally test the example models, the data described above for the 230 samples was split into training, validation, and testing datasets.

To ensure that no peptides appeared in more than one of the training, validation, and testing datasets, the following procedure was performed. First all peptides from the 230 samples that appeared in more than one location in the proteome were removed. Then, the remaining peptides from the 230 samples were partitioned into blocks of 10 adjacent peptides. Each block of adjacent peptides was assigned uniquely to the training dataset, the validation dataset, or the testing dataset. In this way, no peptide appeared in more than one dataset of the training, validation, and testing datasets.

The training dataset included 188.210 peptides presented by MHC class II molecules from 226 of the 230 total samples. The 188,210 peptides included in the training dataset were between lengths of 9 and 20 amino acids, inclusive. The Bi-LSTM model and the Inception model were each trained on the training dataset using the ADAM optimizer and early stopping.

The validation dataset included 21,764 peptides presented by MHC class II molecules from the same 226 samples used in the training dataset. The validation dataset was used only for early stopping.

The testing dataset included peptides presented by MHC class II molecules that were identified from a tumor sample using mass spectrometry. Specifically, the testing dataset comprised 232 peptides that were identified from four tumor samples. The peptides included in the testing dataset were held out of the training dataset as described above.

Following training and validation of the Bi-LSTM and Inception models using the training dataset and the validation dataset, respectively, the models were tested using the testing dataset. Performance of the Bi-LSTM and Inception models on the testing dataset is depicted in FIG. 14Q as full precision-recall curves and AUC scores. As shown in FIG. 14Q, the Inception model outperformed the Bi-LSTM model, and achieved an AUC of 0.347. The Bi-LSTM model achieved an AUC of 0.238.

### XI.A.4. Example 4

To further evaluate whether the prediction model disclosed herein can be applied to class I HLA peptide presentation, published class II mass spectrometry data was obtained for two cell lines, each of which expressed a single HLA class I allele. One cell line expressed HLA-DRB1*15:01 and the other expressed HLA-DRB5*01:01¹⁵⁰. These two cell lines were used for training data. For test data, class II mass spectrometry data was obtained from a separate cell line expressing both HLA-DRB1*15:01 and HLA-DRB5*01:01.¹⁵¹ RNA sequencing data was not available either the training or testing cell lines, therefore RNA-sequencing data from a different B-cell line, B721.221⁹², was substituted.

The peptide sets were split into training, validation and testing sets using the same procedure as for the HLA class I data, except that for the class II data peptides with lengths between 9 and 20 were included. The training data included 330 peptides presented by HLA-DRB1*15:01, and 103 peptides presented by HLA-DRB5*01:01. The test dataset included 223 peptides presented by either HLA-DRB1*15:01 or HLA-DRB5*01:01 along with 4708 non-presented peptides.

We trained an ensemble of 10 models on the training dataset to predict HLA class II peptide presentation. The architecture and training procedures for these models were identical to those used to predict class I presentation, with the exception that class II models took as input peptides sequences one hot-encoded and zero-padded to length 20 rather than 11.

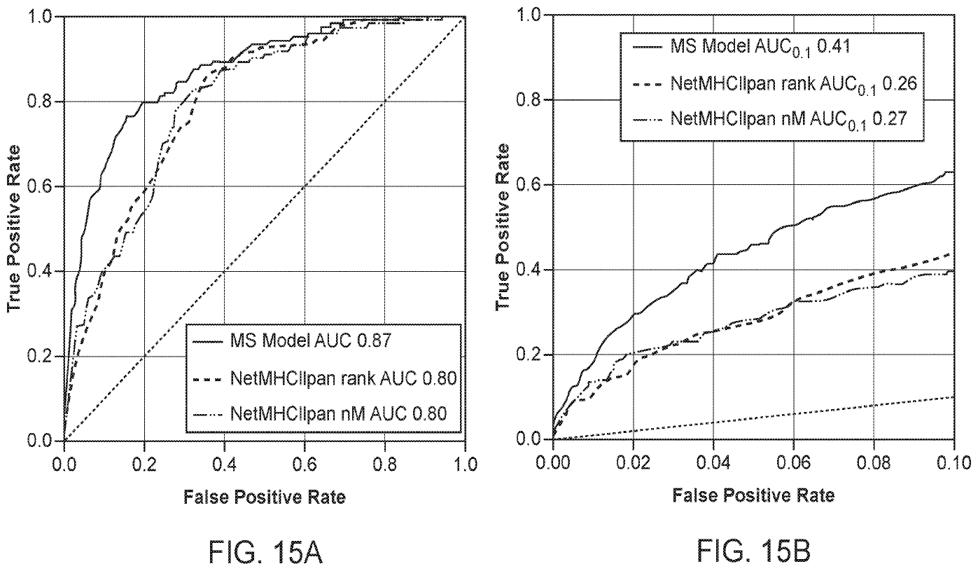

FIG. 15 compares the predictive performance of the the “MS Model,” “NetMHCIIpan rank”: NetMHCIIpan 3.1¹⁵², taking the lowest NetMHCIIpan percentile rank across HLA-DRB1*15:01 and HLA-DRB5*01:01, and “NetMHCIIpan nM”: NetMHCIIpan 3.1, taking the strongest affinity in nM units across HLA-DRB1*15:01 and HLA-DRB5*01:01, at ranking the peptides in the HLA-DRB1*15:01/HLA-DRB5*01:01 test dataset. The “MS Model” is the MHC class II presentation prediction model disclosed herein.

Specifically, FIG. 15 depicts receiver operating characteristic (ROC) curves and the area under the ROC curve AUC (panel A) and AUC_{0.1} (panel B) statistics for these ranking methods. AUC_{0.1} is AUC between 0 and 0.1FPR*10, commonly considered in the epitope prediction field¹⁹. The NetMHCIIpan nM and rank methods performed similarly. The MS model performed best, significantly exceeding performance of the comparator methods, particularly in the critical high-specificity region of the ROC curve (AUC_{0.1} 0.41 vs. 0.27).

### XIII.B. Example of Presentation Model Parameters Determined for MHC Class II Alleles

The following shows a set of parameters determined for a variation of the multi-allele presentation model (equation (16)) generating implicit per-allele presentation likelihoods for class II MHC alleles HLA-DRB1*12:01 and HLA-DRB1*10:01:

u=expit(relu(X·W¹+b¹)·W²+b²),

where relu(⋅) is the rectified linear unit (RELU) function, W¹, b¹, W², and b² are the set of parameters θ determined for the model. The allele-interacting variables X are contained in a 1×399) matrix consisting of 1 row of one-hot encoded and middle-padded peptide sequences per input peptide. The dimensions of W¹ are (399×256), the dimensions of b¹ (1×256), the dimensions of W² are (256×2), and b² are (1×2). The first column of the output indicates the implicit per-allele probability of presentation for the peptide sequence by the allele HLA-DRB1*12:01, and the second column of the output indicates the implicit per-allele for the peptide sequence by the allele HLA-DRB1*10:01. For demonstration purposes, values for b¹, b², W¹, and W² are listed in Appendix A

### XIII. Example 9: MHC Class II Presentation Model Evaluation of T-Cell Data

To evaluate whether accurate prediction of peptide presentation by MHC class II alleles translates into the ability to identify human tumor CD4 T-cell epitopes (i.e., immunotherapy targets), published CD4+ T-cell multimer/tetramer assay data were downloaded from the Immune Epitope Database (IEDB)⁸⁸. These data consisted of 3,470 peptides of length 9-20 residues from human samples with 18 distinct HLA-DRB alleles, including 14 HLA-DRB1 alleles, 2 HLA-DRB3 alleles, 1 HLA-DRB4 allele, and 1 HLA-DRB5 allele. On average, each allele had 33 samples that contained that allele. The full MHC Class II MS model (the same model described above in Section XII.A.2) was compared against the binding affinity predictor NetMHCII 2.3. Across the 18 alleles, the full MHC Class II MS model had an average ROC area under the curve (ROC AUC) of 0.81 with a standard deviation of 0.08, whereas the NetMHCII 2.3 model had a ROC AUC of just 0.65 with a larger standard deviation of 0.13. These results demonstrate the superior ability of the full MHC Class II MS model to predict CD4 T-cell epitopes. On a per-allele basis, for some of the more common alleles, like HLA-DRB1*01:01, the ROC AUC was much more similar between the two models. For example, for the HLA-DRB1*01:01 allele, the full MHC Class II MS model had ROC AUC of 0.83 and the NetMHCII 2.3 model had ROC AUC of 0.81. However, most alleles had a much wider spread in performance between the two models. Of the 18 per-allele tests, the full MHC Class II MS model outperformed the NetMHCII 2.3 model on 17 alleles. In only one allele. HLA-DRB1*15:02, did the NetMHCII 2.3 outperform the full MHC Class II MS model. However, this allele was not well represented in the full MHC Class II MS model's training data, which included only one sample containing that allele.

### XIV. Example 10: MHC Class II Presentation Model Evaluation of Retrospective Neoantigen T-Cell Data

This example further evaluates whether accurate prediction of peptide presentation by MHC class II molecules translates into the ability to identify human tumor CD4 T-cell epitopes. To perform this evaluation, the CD4+ immunogenicities of peptides predicted by MHC class II presentation models were ranked.

The appropriate test dataset for this evaluation includes peptides that are presented by the MHC class II molecules on the tumor cell surface and are recognized by T-cells. In addition, formal performance assessment required not only positive-labeled (i.e., T-cell recognized) peptides, but also a sufficient number of negative-labeled (i.e., tested but not T-cell recognized) peptides. Mass spectrometry datasets address tumor presentation, but not T-cell recognition. Oppositely, priming or T-cell assays post-vaccination address the presence of T-cell precursors and T-cell recognition, but not tumor presentation. For example, a strong HLA-binding peptide whose source gene is expressed at low level in the tumor could give rise to a strong CD4 T-cell response post-immunization that would not be therapeutically useful because the peptide is not presented by the tumor.

To obtain an appropriate test dataset for this evaluation, published data was collected from recent studies.¹⁶³ The collected test dataset included 69 positive-labeled single nucleotide variant (SNV) mutations CD4+ reactive to TILs, across 45 patients. As mentioned above, the collected test dataset also included negative-labeled SNV mutations. Specifically, there was a mean of 104 and a median of 106 negative-labeled SNV mutations per patient.

Each SNV mutation in the test dataset was represented as a sequence of 25 amino acids, with the SNV mutation in the middle of the sequence at amino acid position 13. For each sequence of 25 amino acids, all possible peptides of lengths between 9 and 20 amino acids containing the SNV mutation were then generated. Each sequence of 25 amino acids yielded 118 possible peptides. For each possible peptide, flanking sequences of 5 amino acids were added on the left and on the right of the peptide.

To simulate the selection of antigens for personalized immunotherapy, the SNV mutations of each patient in the test dataset were ranked in order of likelihood of presentation by MHC class II alleles of the patient, using the Inception model disclosed herein and the NetMHCIIPan 3.2 binding affinity model, with a gene expression threshold of TPM=1. The Inception model used was trained to predict peptide presentation by 32 different MHC class II alleles, which covered 25 of 30 MHC class II alleles present in patients in the test dataset.

For the Inception model, to calculate a likelihood of presentation of each SNV mutation of each patient, a presentation score for each of the 118 possible peptides for the SNV mutation, for each of the patient's identified MHC class II alleles, was determined using the Inception model. Then, the highest presentation score determined by the Inception model for each of the patient's MHC class II alleles was identified. Finally, these highest presentation scores for each of the patient's MHC class II alleles were summed to determine the overall likelihood of presentation for the SNV mutation of the patient.

For the NetMHCIIPan 3.2 model, to calculate a likelihood of presentation of each SNV mutation of each patient, a binding affinity of each of the 118 possible peptides for the SNV mutation, for each of the patient's identified MHC class II alleles, was determined using the NetMHCIIPan 3.2 model. Then, the highest inverse binding affinity determined by the NetMHCIIPan 3.2 model for each of the patient's MHC class II alleles was identified. Note that the highest inverse binding affinities were identified because a lower binding affinity indicates a greater likelihood of presentation. Finally, these highest inverse binding affinities for each of the patient's MHC class II alleles were summed to determine the overall likelihood of presentation for the SNV mutation of the patient.

Next, the SNV mutations of each patient were ranked in order of likelihood of presentation by MHC class II alleles of the patient, as determined by both the Inception model and the NetMHCIIPan 3.2 model. As antigen-specific immunotherapies are technically limited in the number of MHC class II specificities targeted (e.g., current personalized vaccines encode ˜10-20 somatic mutations⁸⁰⁻⁸², ˜10 of which can be MHC class II specific), the top 1,2,3,4,5, and 10 SNV mutations for each patient were ranked.

Additionally, as a control, for each patient, each of the patient's SNV mutations originating from a gene having TPM>=1 was randomly ranked. Specifically, for each patient, each of the patient's SNV mutations originating from a gene having TPM>=1 was randomly ranked for 100 trials, to determine an overall ranking of each SNV mutation of each patient.

Following ranking of the SNV mutations, the predictive models were compared by counting the number of pre-existing T-cell responses in the top 1,2,3,4,5, and 10-ranked SNV mutations for each patient with at least one pre-existing T-cell response. Then, the proportion of SNV mutations recognized by T-cells (e.g., pre-existing T-cell responses) for the top 1, 2, 3, 4, 5, and 10-ranked SNV mutations identified by the different models for each patient with at least one pre-existing T-cell response, were compared. Specifically, Table 2 below depicts the percentage of positive-labeled SNV mutations out of the total 69 positive-labeled SNV mutations predicted by the given model in the top 1,2,3,4,5 and 10 predictions. As shown in Table 2, the Inception model is more likely than the NetMHCIIPan 3.2 model and the random predictions to accurately predict CD4+ immunogenic, MHC class II presented peptides.

TABLE 2

| Model | Top 1 | Top 2 | Top 3 | Top 4 | Top 5 | Top 10 |
|---|---|---|---|---|---|---|
| Inception | 9% | 17% | 17% | 19% | 20% | 32% |
| NetMHCIIPan 3.2 | 9% | 12% | 16% | 18% | 19% | 29% |
| Random | 1% | 3% | 5% | 6% | 8% | 16% |

Therefore, this evaluation establishes the superior ability of the Inception model to identify not just neoantigens that are able to prime T-cells as in previous literature^{81,82,97}, but—more stringently—neoantigens presented to T-cells by tumors.

### XV. Example 11: Prospective Identification of Neoantigen-Reactive T-Cells in Cancer Patients

This prospective example will demonstrate that improved prediction can enable neoantigen identification from routine patient samples. To do so, archival FFPE tumor biopsies and 5-30 ml of peripheral blood will analyzed from patients with metastatic NSCLC undergoing anti-PD(L)1 therapy. Somatic mutations (SNVs and short indels) will be identified for each patient using tumor whole exome sequencing, tumor transcriptome sequencing, and matched normal exome sequencing. The MHC class II full MS model will be applied to prioritize 20 neoepitopes per patient for testing against pre-existing anti-tumor T-cell responses. To focus the analysis on likely CD4 responses, the prioritized peptides will be synthesized as 8-11mer minimal epitopes (Methods), and then peripheral blood mononuclear cells (PBMCs) will be cultured with the synthesized peptides in short in vitro stimulation (IVS) cultures to expand neoantigen-reactive T-cells. After two weeks the presence of antigen-specific T-cells will be assessed using IFN-gamma ELISpot against the prioritized neoepitopes. In patients for whom sufficient PBMCs re available, separate experiments will also performed to fully or partially deconvolve the specific antigens recognized.

First, T-cell responses to patient-specific neoantigen peptide pools will be detected for each of the patients. For each patient, predicted neoantigens will be combined into 2 pools of peptides each according to model ranking and any sequence homologies (homologous peptides will be separated into different pools). Then, for each patient, the in vitro expanded PBMCs for the patient will be stimulated with the 2 patient-specific neoantigen peptide pools in IFN-gamma ELISpot. DMSO negative controls and PHA positive controls will also be conducted to detect background and T-cell viability, respectively. Samples with values >2-fold increase above background will be considered positive, responsive patients. Furthermore, to verify that in vitro culture conditions expand only pre-existing in vivo primed memory T-cells, rather than enabling de novo priming in vitro, a series of control experiments will be performed with neoantigens in HLA-matched healthy donors. It is expected that pre-existing neoantigen-reactive T-cells will be identified in the majority of patients tested with patient-specific peptide pools using IFN-gamma ELISpot. Additionally, it is expected that a majority of patients will respond to at least one of the tested neoantigen peptides.

### XV.A. Peptides

Custom-made, recombinant lyophilized peptides will be purchased and reconstituted at 10-50 mM in sterile DMSO, aliquoted and stored at −80° C.

### XV.B. Human Peripheral Blood Mononuclear Cells (PBMCs)

Cryopreserved HLA-typed PBMCs from healthy donors (that are confirmed HIV, HCV and HBV seronegative) will be purchased and stored in liquid nitrogen until use. Fresh blood samples and leukopaks will also be purchased and PBMCs will be isolated by Ficoll-Paque density gradient prior to cryopreservation. Patient PBMCs will be processed at local clinical processing centers according to local clinical standard operating procedures (SOPs) and IRB approved protocols. Approving IRBs will include Quorum Review IRB, Comitato Etico Interaziendale A.O.U. San Luigi Gonzaga di Orbassano, and Comitë Ëtico de la Investigacion del Grupo Hospitalario Quiróń en Barcelona.

PBMCs will be isolated through density gradient centrifugation, washed, counted, and cryopreserved in CryoStor CS10 at 5×10⁶ cells/ml. Cryopreserved cells will be shipped in cryoports and transferred to storage in LN₂ upon arrival. Cryopreserved cells will b thawed and washed twice in OpTmizer T-cell Expansion Basal Medium with Benzonase and once without Benzonase. Cell counts and viability will be assessed using the Guava ViaCount reagents and module on the Guava easyCyte HT cytometer (EMD Millipore). Cells will subsequently be re-suspended at concentrations and in media appropriate for proceeding assays (see next sections).

### XV.C. In Vitro Stimulation (IVS) Cultures

Pre-existing T-cells from healthy donor or patient samples will be expanded in the presence of cognate peptides and IL-2 in a similar approach to that applied by Ott et al.⁸¹ Briefly, thawed PBMCs will be rested overnight and stimulated in the presence of peptide pools (10 μM per peptide) in ImmunoCult™-XF T-cell Expansion Medium with 10 IU/ml rhIL-2 for 14 days in 24-well tissue culture plates. Cells will be seeded at 2×10⁶ cells/well and fed every 2-3 days by replacing ⅔ of the culture media.

### XV.D. IFNγ Enzyme Linked Immunospot (ELISpot) Assay

Detection of IFNγ-producing T-cells will be performed by ELISpot assay¹⁴². Briefly, PBMCs (ex vivo or post in vitro expansion) will be harvested, washed in serum free RPMI and cultured in the presence of controls or cognate peptides in OpTmizer T-cell Expansion Basal Medium (ex vivo) or in ImmunoCult™-XF T-cell Expansion Medium (expanded cultures) in ELISpot Multiscreen plates coated with anti-human IFNγ capture antibody. Following 18 hours of incubation in a 5% CO₂, 37° C., humidified incubator, cells will be removed from the plate and membrane-bound IFNγ will be detected using anti-human IFNγ detection antibody, Vectastain Avidin peroxidase complex and AEC Substrate. ELISpot plates will be allowed to dry, stored protected from light, and sent away for standardized evaluation¹⁴³.

### XV.E. Granzyme B ELISA and MSD Multiplex Assay

Detection of secreted IL-2, IL-5 and TNF-alpha in ELISpot supernatants will be performed using a 3-plex assay MSD U-PLEX Biomarker assay (catalog number K15067L-2). Assays will be performed according to the manufacturer's instructions. Analyte concentrations (pg/ml) will be calculated using serial dilutions of known standards for each cytokine. Detection of Granzyme B in ELISpot supernatants will be performed using the Granzyme B DuoSet® ELISA according to the manufacturer's instructions. Briefly, ELISpot supernatants will be diluted 1:4 in sample diluent and run alongside serial dilutions of Granzyme B standards to calculate concentrations (pg/ml).

### XV.F. Negative Control Experiments for IVS Assay—Neoantigens from Tumor Cell Lines Tested in Healthy Donors

Negative control experiments for IVS assay for neoantigens from tumor cell lines tested in healthy donors will be performed. In such experiments, healthy donor PBMCs will be stimulated in IVS culture with peptide pools containing positive control peptides (previous exposure to infectious diseases), HLA-matched neoantigens originating from tumor cell lines (unexposed), and peptides derived from pathogens for which the donors are seronegative. Expanded cells will be subsequently analyzed by IFNγ ELISpot (105 cells/well) following stimulation with DMSO (negative controls), PHA and common infectious diseases peptides (positive controls), neoantigens (unexposed), or HIV and HCV peptides (donors will be confirmed to be seronegative).

### XV.G. Negative Control Experiments for IVS Assay—Neoantigens from Patients Tested in Healthy Donors

Negative control experiments for IVS assay for neoantigens from patients tested for reactivity in healthy donors will be performed. Specifically, assessment of T-cell responses in healthy donors to HLA-matched neoantigen peptide pools will be performed. Healthy donor PBMCs will be stimulated with controls (DMSO, CEF and PHA) or HLA-matched patient-derived neoantigen peptides in ex vivo IFN-gamma ELISpot. Additionally, healthy donor PBMCs post IVS culture, expanded in the presence of either neoantigen pool or CEF pool will be stimulated in IFN-gamma ELISpot either with controls (DMSO, CEF and PHA) or HLA-matched patient-derived neoantigen peptide pool.

### XVI. Methods of Examples 8-11

The below methods of Examples 8-11 are discussed in future tense because they will be used in executing the future, prospective Examples 10-11. However, despite the future tense used to describe the below methods, these methods have also been used in the past in the execution of Examples 8 and 9.

### XVI.A. Mass Spectrometry

### XVI.A.1. Specimens

Archived frozen tissue specimens for mass spectrometry analysis will be obtained from commercial sources. A subset of specimens will be also collected prospectively from patients.

### XVI.A.2. HLA Immunoprecipitation

Isolation of HLA-peptide molecules will be performed using established immunoprecipitation (IP) methods after lysis and solubilization of the tissue sample^{87,124-26}. Fresh frozen tissue will be pulverized, lysis buffer (1% CHAPS, 20 mM Tris-HCl, 150 mM NaCl, protease and phosphatase inhibitors, pH=8) will be added to solubilize the tissue and the resultant solution will be centrifuged at 4 C for 2 hours to pellet debris. The clarified lysate will be used for the HLA specific IP. Immunoprecipitation will be performed as previously described using the antibody W6/32¹²⁷. The lysate will be added to the antibody beads and rotated at 4 C overnight for the immunoprecipitation. After immunoprecipitation, the beads will be removed from the lysate. The IP beads will be washed to remove non-specific binding and the HLA/peptide complex will be eluted from the beads with 2N acetic acid. The protein components will be removed from the peptides using a molecular weight spin column. The resultant peptides will be taken to dryness by SpeedVac evaporation and stored at −20 C prior to MS analysis.

### XVI.A.3. Peptide Sequencing

Dried peptides will be reconstituted in HPLC buffer A and loaded onto a C-18 microcapillary HPLC column for gradient elution in to the mass spectrometer. A gradient of 0-40% B (solvent A—0.1% formic acid, solvent B— 0.1% formic acid in 80% acetonitrile) in 180 minutes will be used to elute the peptides into the Fusion Lumos mass spectrometer. MS1 spectra of peptide mass/charge (m/z) will be collected in the Orbitrap detector with 120,000 resolution followed by 20 MS2 low resolution scans collected in the either the Orbitrap or ion trap detector after HCD fragmentation of the selected ion. Selection of MS2 ions will be performed using data dependent acquisition mode and dynamic exclusion of 30 seconds after MS2 selection of an ion. Automatic gain control (AGC) for MS1 scans will be set to 4×105 and for MS2 scans will be set to 1×104. For sequencing HLA peptides, +1, +2 and +3 charge states can be selected for MS2 fragmentation.

MS2 spectra from each analysis will be searched against a protein database using Comet^{128,129} and the peptide identification will be scored using Percolator¹³⁰⁻¹³².

### XVI.B. Machine Learning

### XVI.B.1. Data Encoding

For each sample, the training data points will be all 8-11mer (inclusive) peptides from the reference proteome that mapped to exactly one gene expressed in the sample. The overall training dataset will be formed by concatenating the training datasets from each training sample. Lengths 8-11 will be chosen as this length range captures ˜95% of all HLA class I presented peptides; however, adding lengths 12-15 to the model could be accomplished using the same methodology, at the cost of a modest increase in computational demands. Peptides and flanking sequence will be vectorized using a one-hot encoding scheme. Peptides of multiple lengths (8-11) will be represented as fixed-length vectors by augmenting the amino acid alphabet with a pad character and padding all peptides to the maximum length of 11. RNA abundance of the source protein of the training peptides will be represented as the logarithm of the isoform-level transcripts per million (TPM) estimate obtained from RSEM¹³³. For each peptide, the per-peptide TPM will be computed as the sum of the per-isoform TPM estimates for each of the isoforms that contain the peptide. Peptides from genes expressed at 0 TPM will be excluded from the training data, and at test time, peptides from non-expressed genes are assigned a probability of presentation of 0. Lastly, each peptide will be assigned to an Ensembl protein family ID, and each unique Ensembl protein family ID will correspond to a per-gene presentation propensity intercept (see next section).

### XVI.B.2. Specification of the Model Architecture

The full presentation model has the following functional form:

Pr(peptide i presented)=Σₖ₌₁^{m}aₖⁱ·Pr(peptide i presented by allele a), (Equation I)

where k indexes HLA alleles in the dataset, which run from 1 to m, and aₖⁱ is an indicator variable whose value is 1 if allele k is present in the sample from which peptide i is derived and 0 otherwise. Note that for a given peptide i, all but at most 6 of the aₖⁱ (the 6 corresponding to the HLA type of the sample of origin of peptide i) will be zero. The sum of probabilities will be clipped at 1−ϵ, with ϵ=10⁻⁶ for instance.

The per-allele probabilities of presentation will be modeled as below:

Pr(peptide i presented by allele a)=sigmoid{NNₐ(peptideᵢ)+NN_{flanking}(flankingᵢ)+NN_{RNA}(log(TPMᵢ))+αₛₐₘₚₗₑ₍ᵢ₎+βₚᵣₒₜₑᵢₙ₍ᵢ₎},

where the variables have the following meanings: sigmoid is the sigmoid (aka expit) function, peptideᵢ is the one hot-encoded middle-padded amino acid sequence of peptide i, NNₐ is a neural network with linear last-layer activation modeling the contribution of the peptide sequence to the probability of presentation, flankingᵢ is the one hot-encoded flanking sequence of peptide i in its source protein, NN_{flanking} is a neural network with linear last-layer activation modeling the contribution of the flanking sequence to the probability of presentation, TPMᵢ is the expression of the source mRNAs of peptide i in TPM units, sample(i) is the sample (i.e., patient) of origin of peptide i, αₛₐₘₚₗₑ₍ᵢ₎ is a per-sample intercept, protein(i) is the source protein of peptide i, and βₚᵣₒₜₑᵢₙ₍ᵢ₎ is a per-protein intercept (aka the per-gene propensity of presentation).

The component neural networks of the models will have the following architectures:
Each of the NNₐ is one output node of a one-hidden-layer multi-layer-perceptron (MLP) with input dimension 231 (11 residues×21 possible characters per residue, including the pad character), width 256, rectified linear unit (ReLU) activations in the hidden layer, linear activation in the output layer, and one output node per HLA allele a in the training dataset.
NN_{flanking} is a one-hidden-layer MLP with input dimension 210 (5 residues of N-terminal flanking sequence+5 residues of C-terminal flanking sequence×21 possible characters per residue, including the pad character), width 32, rectified linear unit (ReLU) activations in the hidden layer and linear activation in the output layer.
NN_{RNA} is a one-hidden-layer MLP with input dimension 1, width 16, rectified linear unit (ReLU) activations in the hidden layer and linear activation in the output layer.

Note that some components of the model (e.g., NNₐ) depend on a particular HLA allele, but many components (NN_{flanking}, NN_{RNA}, αₛₐₘₚₗₑ₍ᵢ₎, βₚᵣₒₜₑᵢₙ₍ᵢ₎) do not. The former is referred to as “allele-interacting” and the latter as “allele-noninteracting”. Features to model as allele-interacting or noninteracting will be chosen on the basis of biological prior knowledge: the HLA allele sees the peptide, so the peptide sequence will be modeled as allele-interacting, but no information about the source protein, RNA expression or flanking sequence is conveyed to the HLA molecule (as the peptide has been separated from its source protein by the time it encounters the HLA in the endoplasmic reticulum), so these features will be modeled as allele-noninteracting. The model will be implemented in Keras v2.0.4¹³⁴ and Theano v0.9.0¹³⁵.

The peptide MS model will use the same deconvolution procedure as the full MS model (Equation 1), but the per-allele probabilities of presentation will be generated using reduced per-allele models that consider only peptide sequence and HLA allele:

Pr(peptide i presented by allele a)=sigmoid{NNₐ(peptideᵢ)}.

The peptide MS model will use the same features as binding affinity prediction, but the weights of the model will be trained on a different data type (i.e., mass spectrometry data vs HLA-peptide binding affinity data). Therefore, comparing the predictive performance of the peptide MS model to the full MS model will reveal the contribution of non-peptide features (i.e., RNA abundance, flanking sequence, gene ID) to the overall predictive performance, and comparing the predictive performance of the peptide MS model to the binding affinity models will reveal the importance of improved modeling of the peptide sequence to the overall predictive performance.

### XVI.B.3. Train/Validate/Test Splits

No peptides will appear in more than one of the training/validation/testing sets by using the following procedure: first all peptides will be removed from the reference proteome that appear in more than one protein, then the proteome will be partitioned into blocks of 10 adjacent peptides. Each block will be assigned uniquely to the training, validation, or testing sets. In this way, no peptide will appear in more than one of the training, validation, or testing sets. The validation set will be used only for early stopping. Peptides from single-allele samples will be included in the training data, but the set of peptides (both presented and non-presented) incorporated into the training and validation sets will be disjoint from the set of peptides used as test data.

### XVI.B.4. Model Training

For model training, all peptides will be modeled as independent where the per-peptides loss is the negative Bernoulli log-likelihood loss function (aka log loss). Formally, the contribution of peptide i to the overall loss is

Loss(i)=−log (Bernoulli(yᵢ|Pr(peptide i presented))),

where yᵢ is the label of peptide i; i.e., yᵢ=1 if peptide i is presented and 0 otherwise, and Bernoulli(y|p) detnoes the Bernoulli likelihood of parameter p∈[0, 1] given i.i.d. binary observation vector y. The model will be trained by minimizing the loss function.

In order to reduce training time, the class balance will be adjusted by removing 90% of the negative-labeled training data at random. Model weights will be initialized using the Glorot uniform procedure 61 and trained using the ADAM62 stochastic optimizer with standard parameters on Nvidia Maxwell TITAN X GPUs. A validation set consisting of 10% of the total data will be used for early stopping. The model will be evaluated on the validation set every quarter-epoch and model training will be stopped after the first quarter-epoch where the validation loss (i.e., the negative Bernoulli log-likelihood on the validation set) fails to decrease.

The full presentation model will be an ensemble of 10 model replicates, with each replicate trained independently on a shuffled copy of the same training data with a different random initialization of the model weights for every model within the ensemble. At test time, predictions will be generated by taking the mean of the probabilities output by the model replicates.

### XVI.B.5. Motif Logos

Motif logos will be generated using the weblogolib Python API v3.5.0¹³⁸. To generate binding affinity logos, the mhc_ligand_full.csv file will be downloaded from the Immune Epitope Database (IEDB⁸⁸) and peptides meeting the following criteria will be retained: measurement in nanomolar (nM) units, reference date after 2000, object type equal to “linear peptide” and all residues in the peptide drawn from the canonical 20-letter amino acid alphabet. Logos will be generated using the subset of the filtered peptides with measured binding affinity below the conventional binding threshold of 500 nM. For alleles pair with too few binders in IEDB, logos will not be generated. To generate logos representing the learned presentation model, model predictions for 2,000,000 random peptides will be predicted for each allele and each peptide length. For each allele and each length, the logos will be generated using the peptides ranked in the top 1% (i.e., the top 20,000) by the learned presentation model. Importantly, this binding affinity data from IEDB will not be used in model training or testing, but rather used only for the comparison of motifs learned.

### XVI.B.6. Binding Affinity Prediction

We will predict peptide-MHC binding affinities using the binding affinity-only predictor from NetMHCII 2.3, an open-source, GPU-compatible HLA class I binding affinity predictor. To combine binding affinity predictions for a single peptide across multiple HLA alleles, the minimum binding affinity will be selected. To combine binding affinities across multiple peptides (i.e., in order to rank mutations spanned by multiple mutated peptides), the minimum binding affinity across the peptides will be selected. For RNA expression thresholding on the T-cell dataset, tumor-type matched RNA-seq data from TCGA to threshold at TPM>1 will be used. All of the original T-cell datasets will be filtered on TPM>0 in the original publications, so the TCGA RNA-seq data to filter on TPM>0 will not be used.

### XVI.B.7. Presentation Prediction

To combine probabilities of presentation for a single peptide across multiple HLA alleles, the sum of the probabilities will be identified, as in Equation 1. To combine probabilities of presentation across multiple peptides (i.e., in order to rank mutations spanned by multiple peptides), the sum of the probabilities of presentation will be identified. Probabilistically, if presentation of the peptides is viewed as i.i.d. Bernoulli random variables, the sum of the probabilities corresponds to the expected number of presented mutated peptides:

$$E [ \# presented neoantigens spanning mutation i ] = {\sum }_{j = 1}^{{n}_{i}} Pr [ epitope j presented ] ,$$

where Pr[epitope j presented] is obtained by applying the trained presentation model to epitope j, and nᵢ denotes the number of mutated epitopes spanning mutation i. For example, for an SNV i distant from the termini of its source gene, there are 8 spanning 8-mers, 9-spanning 9-mers, 10 spanning 10-mers and 11 spanning 11-mers, for a total of nᵢ=38 spanning mutated epitopes.

### XVI.C. Next Generation Sequencing

### XVI.C.1. Specimens

For transcriptome analysis of the frozen resected tumors, RNA will be obtained from same tissue specimens (tumor or adjacent normal) as used for MS analyses. For neoantigen exome and transcriptome analysis in patients on anti-PD1 therapy, DNA and RNA will be obtained from archival FFPE tumor biopsies. Adjacent normal, matched blood or PBMCs will be used to obtain normal DNA for normal exome and HLA typing.

### XVI.C.2. Nucleic Acid Extraction and Library Construction

Normal/germline DNA derived from blood will be isolated using Qiagen DNeasy columns following manufacturer recommended procedures. DNA and RNA from tissue specimens will be isolated using Qiagen Allprep DNA/RNA isolation kits following manufacturer recommended procedures. The DNA and RNA will be quantitated by Picogreen and Ribogreen Fluorescence (Molecular Probes), respectively. Specimens with >50 ng yield will be advanced to library construction. DNA sequencing libraries will be generated by acoustic shearing followed by DNA Ultra II library preparation kit following the manufacturers recommended protocols. Tumor RNA sequencing libraries will be generated by heat fragmentation and library construction with RNA Ultra II. The resulting libraries will be quantitated by Picogreen (Molecular Probes).

### XVI.C.3. Whole Exome Capture

Exon enrichment for both DNA and RNA sequencing libraries will be performed using xGEN Whole Exome Panel. One to 1.5 μg of normal DNA or tumor DNA or RNA-derived libraries will be used as input and allowed to hybridize for greater than 12 hours followed by streptavidin purification. The captured libraries will be minimally amplified by PCR and quantitated by NEBNext Library Quant Kit. Captured libraries will be pooled at equimolar concentrations and clustered using the c-bot and sequenced at 75 base paired-end on a HiSeq4000 to a target unique average coverage of >500× tumor exome, >100× normal exome, and >100M reads tumor transcriptome.

### XVI.C.4. Analysis

Exome reads (FFPE tumor and matched normals) will be aligned to the reference human genome (hg38) using BWA-MEM¹⁴⁴ (v. 0.7.13-r1126). RNA-seq reads (FFPE and frozen tumor tissue samples) will be aligned to the genome and GENCODE transcripts (v. 25) using STAR (v. 2.5.1b). RNA expression will be quantified using RSEM¹³³ (v. 1.2.31) with the same reference transcripts. Picard (v. 2.7.1) will be used to mark duplicate alignments and calculate alignment metrics. For FFPE tumor samples following base quality score recalibration with GATK¹⁴⁵ (v. 3.5-0), substitution and short indel variants will be determined using paired tumor-normal exomes with FreeBayes¹⁴⁶ (1.0.2). Filters will include allele frequency >4%; median base quality >25, minimum mapping quality of supporting reads 30, and alternate read count in normal <=2 with sufficient coverage obtained. Variants will also be detected on both strands. Somatic variants occurring in repetitive regions will be excluded. Translation and annotation will be performed with snpEff¹⁴⁷ (v. 4.2) using RefSeq transcripts. Non-synonymous, non-stop variants verified in tumor RNA alignments will be advanced to neoantigen prediction. Optitype¹⁴⁸ 1.3.1 will be used to generate HLA types.

### XVI.C.5. Tumor Cell Lines and Matched Normals for IVS Control Experiments

Tumor cell lines and their normal donor matched control cell lines will be all purchased and grown to 10⁸³-10⁸⁴ cells per seller's instructions, then snap frozen for nucleic acid extraction and sequencing. NGS processing will be performed generally as described above, except that MuTect¹⁴⁹ (3.1-0) will be used for substitution mutation detection only.

### XVII. Example 12: Prospective Sequencing TCRs of Neoantigen-Specific Memory T-Cells from Peripheral Blood of a NSCLC Patient

TCRs of neoantigen-specific memory T-cells will then be sequenced from the peripheral blood of a NSCLC patient. Peripheral blood mononuclear cells (PBMCs) from a NSCLC patient will be collected after ELISpot incubation. Specifically, in vitro expanded PBMCs from the patient will be stimulated in IFN-gamma ELISpot with patient-specific individual neoantigen peptides, with the patient-specific neoantigen peptide pool, and with DMSO negative control. Following incubation and prior to addition of detection antibody, the PBMCs will be transferred to a new culture plate and maintained in an incubator during completion of the ELISpot assay. Positive (responsive) wells will be identified based on ELISpot results. Cells from positive wells and negative control (DMSO) wells will be combined and stained for CD137 with magnetically-labelled antibodies for enrichment using Miltenyi magnetic isolation columns.

CD137-enriched and -depleted T-cell fractions isolated and expanded as described above will be sequenced using 10× Genomics single cell resolution paired immune TCR profiling approach. Specifically, live T-cells will be partitioned into single cell emulsions for subsequent single cell cDNA generation and full-length TCR profiling (5′ UTR through constant region—ensuring alpha and beta pairing). One approach utilizes a molecularly barcoded template switching oligo at the 5′end of the transcript, a second approach utilizes a molecularly barcoded constant region oligo at the 3′ end, and a third approach couples an RNA polymerase promoter to either the 5′ or 3′ end of a TCR. All of these approaches enable the identification and deconvolution of alpha and beta TCR pairs at the single-cell level. The resulting barcoded cDNA transcripts will undergo an optimized enzymatic and library construction workflow to reduce bias and ensure accurate representation of clonotypes within the pool of cells. Libraries will be sequenced on Illumina's MiSeq or HiSeq4000 instruments (paired-end 150 cycles) for a target sequencing depth of about five to fifty thousand reads per cell. The presence of the TCRa and TCRb chains will be confirmed by an orthogonal anchor-PCR based TCR sequencing approach (Archer). This particular approach has the advantage of using limited cell numbers as input and fewer enzymatic manipulations when compared to the 10× Genomics based TCR sequencing.

Sequencing outputs will be analyzed using the 10× software and custom bioinformatics pipelines to identify T-cell receptor (TCR) alpha and beta chain pairs. Clonotypes will be defined as alpha, beta chain pairs of unique CDR3 amino acid sequences. Clonotypes will be filtered for single alpha and single beta chain pairs present at frequency above 2 cells to yield a final list of clonotypes per target peptide in the patient.

In summary, using the method described above, memory CD4+ T-cells from the peripheral blood of the patient, that are neoantigen-specific to the patient's tumor neoantigens identified as discussed above with regard to Example 11 in Section XV., will be identified. The TCRs of these identified neoantigen-specific T-cells will be sequenced. And furthermore, sequenced TCRs that are neoantigen-specific to the patient's tumor neoantigens as identified by the above presentation models, will be identified.

### XVIII. Example 13: Use of Neoantigen-Specific Memory T-Cells for T-Cell Therapy

After T-cells and/or TCRs that are neoantigen-specific to neoantigens presented by a patient's tumor are identified, these identified neoantigen-specific T-cells and/or TCRs can be used for T-cell therapy in the patient. Specifically, these identified neoantigen-specific T-cells and/or TCRs can be used to produce a therapeutic quantity of neoantigen-specific T-cells for infusion into a patient during T-cell therapy. Two methods for producing a therapeutic quantity of neoantigen specific T-cells for use in T-cell therapy in a patient are discussed herein in Sections XVIII.A. and XVIII.B. The first method comprises expanding the identified neoantigen-specific T-cells from a patient sample (Section XVIII.A.). The second method comprises sequencing the TCRs of the identified neoantigen-specific T-cells and cloning the sequenced TCRs into new T-cells (Section XVIII.B.). Alternative methods for producing neoantigen specific T-cells for use in T-cell therapy that are not explicitly mentioned herein can also be used to produce a therapeutic quantity of neoantigen specific T-cells for use in T-cell therapy. Once the neoantigen-specific T-cells are obtained via one or more of these methods, these neoantigen-specific T-cells may be infused into the patient for T-cell therapy.

### XVIII.A. Identification and Expansion of Neoantigen-Specific Memory T-Cells from a Patient Sample for T-Cell Therapy

A first method for producing a therapeutic quantity of neoantigen specific T-cells for use in T-cell therapy in a patient comprises expanding identified neoantigen-specific T-cells from a patient sample.

Specifically, to expand neoantigen-specific T-cells to a therapeutic quantity for use in T-cell therapy in a patient, a set of neoantigen peptides that are most likely to be presented by a patient's cancer cells are identified using the presentation models as described above. Additionally, a patient sample containing T-cells is obtained from the patient. The patient sample may comprise the patient's peripheral blood, tumor-infiltrating lymphocytes (TIL), or lymph node cells.

In embodiments in which the patient sample comprises the patient's peripheral blood, the following methods may be used to expand neoantigen-specific T-cells to a therapeutic quantity. In one embodiment, priming may be performed. In another embodiment, already-activated T-cells may be identified using one or more of the methods described above. In another embodiment, both priming and identification of already-activated T-cells may be performed. The advantage to both priming and identifying already-activated T-cells is to maximize the number of specificities represented. The disadvantage both priming and identifying already-activated T-cells is that this approach is difficult and time-consuming. In another embodiment, neoantigen-specific cells that are not necessarily activated may be isolated. In such embodiments, antigen-specific or non-specific expansion of these neoantigen-specific cells may also be performed. Following collection of these primed T-cells, the primed T-cells can be subjected to rapid expansion protocol. For example, in some embodiments, the primed T-cells can be subjected to the Rosenberg rapid expansion protocol (https://www.ncbi.nlm.nih.gov/gmc/articles/PMC2978753/, https.//www.ngbi.nlm.nih.gov/pmc/articles/PMC2305721/)^{153,154}.

In embodiments in which the patient sample comprises the patient's TIL, the following methods may be used to expand neoantigen-specific T-cells to a therapeutic quantity. In one embodiment, neoantigen-specific TIL can be tetramer/multimer sorted ex vivo, and then the sorted TIL can be subjected to a rapid expansion protocol as described above. In another embodiment, neoantigen-nonspecific expansion of the TIL may be performed, then neoantigen-specific TIL may be tetramer sorted, and then the sorted TIL can be subjected to a rapid expansion protocol as described above. In another embodiment, antigen-specific culturing may be performed prior to subjecting the TIL to the rapid expansion protocol. (https://www.ncbi.nlm.nih.gov/pmc/articles/PMC4607110/, https://onlinelibrary.wiley.com/doi/pdf/10.1002/eji.201545849)^{155,156}.

In some embodiments, the Rosenberg rapid expansion protocol may be modified. For example, anti-PD1 and/or anti-41BB may be added to the TIL culture to simulate more rapid expansion. (https://jitc.biomedcentral.com/articles/10.1186/s40425-016-0164-7)¹⁵⁷.

### XVIII.B. Identification of Neoantigen-Specific T Cells, Sequencing TCRs of Identified Neoantigen-Specific T Cells, and Cloning of Sequenced TCRs into New T-Cells

A second method for producing a therapeutic quantity of neoantigen specific T-cells for use in T-cell therapy in a patient comprises identifying neoantigen-specific T-cells from a patient sample, sequencing the TCRs of the identified neoantigen-specific T-cells, and cloning the sequenced TCRs into new T-cells.

First, neoantigen-specific T-cells are identified from a patient sample, and the TCRs of the identified neoantigen-specific T-cells are sequenced. The patient sample from which T cells can be isolated may comprise one or more of blood, lymph nodes, or tumors. More specifically, the patient sample from which T cells can be isolated may comprise one or more of peripheral blood mononuclear cells (PBMCs), tumor-infiltrating cells (TILs), dissociated tumor cells (DTCs), in vitro primed T cells, and/or cells isolated from lymph nodes. These cells may be fresh and/or frozen. The PBMCs and the in vitro primed T cells may be obtained from cancer patients and/or healthy subjects.

After the patient sample is obtained, the sample may be expanded and/or primed. Various methods may be implemented to expand and prime the patient sample. In one embodiment, fresh and/or frozen PBMCs may be simulated in the presence of peptides or tandem mini-genes. In another embodiment, fresh and/or frozen isolated T-cells may be simulated and primed with antigen-presenting cells (APCs) in the presence of peptides or tandem mini-genes. Examples of APCs include B-cells, monocytes, dendritic cells, macrophages or artificial antigen presenting cells (such as cells or beads presenting relevant HLA and co-stimulatory molecules, reviewed in https://www.ncbi.nlm.nih.gov/pmc/articles/PMC2929753). In another embodiment, PBMCs, TILs, and/or isolated T-cells may be stimulated in the presence of cytokines (e.g., IL-2, IL-7, and/or IL-15). In another embodiment, TILs and/or isolated T-cells can be stimulated in the presence of maximal stimulus, cytokine(s), and/or feeder cells. In such embodiments, T cells can be isolated by activation markers and/or multimers (e.g., tetramers). In another embodiment, TILs and/or isolated T cells can be stimulated with stimulatory and/or co-stimulatory markers (e.g., CD3 antibodies, CD28 antibodies, and/or beads (e.g., DynaBeads). In another embodiment, DTCs can be expanded using a rapid expansion protocol on feeder cells at high dose of IL-2 in rich media.

Then, neoantigen-specific T cells are identified and isolated. In some embodiments, T cells are isolated from a patient sample ex vivo without prior expansion. In one embodiment, the methods described above with regard to Section XVII, may be used to identify neoantigen-specific T cells from a patient sample. In an alternative embodiment, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T-cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T-cells. Such CD4+ and CD8+ populations can be further sorted into sub-populations by positive or negative selection for markers expressed or expressed to a relatively higher degree on one or more naive, memory, and/or effector T-cell subpopulations.

In some embodiments, CD4+ and CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al. (2012) Blood. 1:72-82: Wang et al. (2012) J Immunother. 35(9):689-701. In some embodiments, combining TCM-enriched CD8+ T-cells and CD4+ T-cells further enhances efficacy.

In embodiments, memory T-cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or ROR1, and positive selection based on a marker characteristic of central memory T-cells, such as CD62L or CCR7, where the positive and negative selections are carried out in either order.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T-cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD1b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immune-magnetic (or affinity-magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained, for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-large T cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See. e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T-cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This can then be diluted 1:1 with media so that the final concentration of DMSO and HSA are 10% and 4%, respectively. Other examples include Cryostor®, CTL-Cryo™ ABC freezing media, and the like. The cells are then frozen to ˜80 degrees C. at a rate of 1 degree per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T-cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T-cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T-cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some embodiments, the PBMC feeder cells are inactivated with Mytomicin C. In some aspects, the feeder cells are added to culture medium prior to the addition of the populations of T-cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T-cells, such as antigen-specific CD4+ T-cells, are obtained by stimulating naive or antigen specific T lymphocytes with antigen. For example, antigen-specific T-cell lines or clones can be generated to cytomegalovirus antigens by isolating T-cells from infected subjects and stimulating the cells in vitro with the same antigen.

In some embodiments, neoantigen-specific T-cells are identified and/or isolated following stimulation with a functional assay (e.g., ELISpot). In some embodiments, neoantigen-specific T-cells are isolated by sorting polyfunctional cells by intracellular cytokine staining. In some embodiments, neoantigen-specific T-cells are identified and/or isolated using activation markers (e.g., CD137, CD38, CD38/HLA-DR double-positive, and/or CD69). In some embodiments, neoantigen-specific CD4+, natural killer T-cells, and/or memory T-cells are identified and/or isolated using class II multimers and/or activation markers. In some embodiments, neoantigen-specific CD4+ T-cells are identified and/or isolated using memory markers (e.g., CD45RA, CD45RO, CCR7, CD27, and/or CD62L). In some embodiments, proliferating cells are identified and/or isolated. In some embodiments, activated T-cells are identified and/or isolated.

After identification of neoantigen-specific T-cells from a patient sample, the neoantigen-specific TCRs of the identified neoantigen-specific T-cells are sequenced. To sequence a neoantigen-specific TCR, the TCR must first be identified. One method of identifying a neoantigen-specific TCR of a T-cell can include contacting the T-cell with an HLA-multimer (e.g., a tetramer) comprising at least one neoantigen; and identifying the TCR via binding between the HLA-multimer and the TCR. Another method of identifying a neoantigen-specific TCR can include obtaining one or more T-cells comprising the TCR; activating the one or more T-cells with at least one neoantigen presented on at least one antigen presenting cell (APC); and identifying the TCR via selection of one or more cells activated by interaction with at least one neoantigen.

After identification of the neoantigen-specific TCR, the TCR can be sequenced. In one embodiment, the methods described above with regard to Section XVII, may be used to sequence TCRs. In another embodiment, TCRa and TCRb of a TCR can be bulk-sequenced and then paired based on frequency. In another embodiment, TCRs can be sequenced and paired using the method of Howie et al., Science Translational Medicine 2015 (doi: 10.1126/scitranslmed.aac5624). In another embodiment, TCRs can be sequenced and paired using the method of Han et al., Nat Biotech 2014 (PMID 24952902, doi 10.1038/nbt.2938). In another embodiment, paired TCR sequences can be obtained using the method described by https://www.biorxiv.org/content/early/2017/05/05/134841 and https://patents.google.com/patent/US20160244825A1/.^{158,159}

In another embodiment, clonal populations of T cells can be produced by limiting dilution, and then the TCRa and TCRb of the clonal populations of T cells can be sequenced. In yet another embodiment, T-cells can be sorted onto a plate with wells such that there is one T cell per well, and then the TCRa and TCRb of each T cell in each well can be sequenced and paired.

Next, after neoantigen-specific T-cells are identified from a patient sample and the TCRs of the identified neoantigen-specific T-cells are sequenced, the sequenced TCRs are cloned into new T-cells. These cloned T-cells contain neoantigen-specific receptors, e.g., contain extracellular domains including TCRs. Also provided are populations of such cells, and compositions containing such cells. In some embodiments, compositions or populations are enriched for such cells, such as in which cells expressing the TCRs make up at least 1, 5, 10, 20, 30, 40, 50, 60, 70, 80, 90, 91, 92, 93, 94, 95, 96, 97, 98, 99, or more than 99 percent of the total cells in the composition or cells of a certain type such as T-cells or CD4+ cells. In some embodiments, a composition comprises at least one cell containing a TCR disclosed herein. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing TCR(s). The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T-cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T-cells or other cell types, such as whole T-cell populations, CD4+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T-cells and/or of CD8+ T-cells are naive T (TN) cells, effector T-cells (TEFF), memory T-cells and sub-types thereof, such as stem cell memory T (TSCM), central memory T (TCM), effector memory T (TEM), or terminally differentiated effector memory T-cells, tumor-infiltrating lymphocytes (TIL), immature T-cells, mature T-cells, helper T-cells, cytotoxic T-cells, mucosa-associated invariant T (MALT) cells, naturally occurring and adaptive regulatory T (Treg) cells, helper T-cells, such as TH1 cells, TH2 cells, TH3 cells, TH17 cells, TH9 cells, TH22 cells, follicular helper T-cells, alpha/beta T-cells, and delta/gamma T-cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

The cells may be genetically modified to reduce expression or knock out endogenous TCRs. Such modifications are described in Mol Ther Nucleic Acids. 2012 December: 1(12): e63; Blood. 2011 Aug. 11:118(6):1495-503; Blood. 2012 Jun. 14; 119(24): 5697-5705: Torikai, Hiroki et al “HLA and TCR Knockout by Zinc Finger Nucleases: Toward “off-the-Shelf” Allogeneic T-Cell Therapy for CD19+ Malignancies.” Blood 116.21 (2010): 3766; Blood. 2018 Jan. 18; 131(3):311-322. doi: 10.1182/blood-2017-05-787598; and WO2016069283, which are incorporated by reference in their entirety.

The cells may be genetically modified to promote cytokine secretion. Such modifications are described in Hsu C, Hughes M S, Zheng Z, Bray R B, Rosenberg S A, Morgan R A. Primary human T lymphocytes engineered with a codon-optimized IL-15 gene resist cytokine withdrawal-induced apoptosis and persist long-term in the absence of exogenous cytokine. J Immunol. 2005; 175:7226-34; Quintarelli C, Vera J F, Savoldo B, Giordano Attianese G M, Pule M, Foster A E, Co-expression of cytokine and suicide genes to enhance the activity and safety of tumor-specific cytotoxic T lymphocytes. Blood. 2007:110:2793-802; and Hsu C, Jones S A, Cohen C J, Zheng Z. Kerstann K. Zhou J, Cytokine-independent growth and clonal expansion of a primary human CD8+ T-cell clone following retroviral transduction with the IL-15 gene. Blood. 2007:109:5168-77.

Mismatching of chemokine receptors on T-cells and tumor-secreted chemokines has been shown to account for the suboptimal trafficking of T-cells into the tumor microenvironment. To improve efficacy of therapy, the cells may be genetically modified to increase recognition of chemokines in tumor micro environment. Examples of such modifications are described in Moon, EKCarpenito, CSun, JWang, LCKapoor, VPredina, J Expression of a functional CCR2 receptor enhances tumor localization and tumor eradication by retargeted human T-cells expressing a mesothelin-specific chimeric antibody receptor. Clin Cancer Res. 2011: 17: 47194730; and Craddock, JALu, ABear, APule, MBrenner, MKRooney, C M et al. Enhanced tumor trafficking of GD2 chimeric antigen receptor T-cells by expression of the chemokine receptor CCR2b. J Immunother. 2010; 33: 780-788.

The cells may be genetically modified to enhance expression of costimulatory/enhancing receptors, such as CD28 and 41BB.

Adverse effects of T-cell therapy can include cytokine release syndrome and prolonged B-cell depletion. Introduction of a suicide/safety switch in the recipient cells may improve the safety profile of a cell-based therapy. Accordingly, the cells may be genetically modified to include a suicide/safety switch. The suicide/safety switch may be a gene that confers sensitivity to an agent, e.g., a drug, upon the cell in which the gene is expressed, and which causes the cell to die when the cell is contacted with or exposed to the agent. Exemplary suicide/safety switches are described in Protein Cell. 2017 August; 8(8): 573-589. The suicide/safety switch may be HSV-TK. The suicide/safety switch may be cytosine daminase, purine nucleoside phosphorylase, or nitroreductase. The suicide/safety switch may be RapaCIDe™, described in U.S. Patent Application Pub. No. US20170166877A1. The suicide/safety switch system may be CD20/Rituximab, described in Haematologica. 2009 September; 94(9): 1316-1320. These references are incorporated by reference in their entirety.

The TCR may be introduced into the recipient cell as a split receptor which assembles only in the presence of a heterodimerizing small molecule. Such systems are described in Science. 2015 Oct. 16; 350(6258): aab4077, and in U.S. Pat. No. 9,587,020, which are hereby incorporated by reference.

In some embodiments, the cells include one or more nucleic acids, e.g., a polynucleotide encoding a TCR disclosed herein, wherein the polynucleotide is introduced via genetic engineering, and thereby express recombinant or genetically engineered TCRs as disclosed herein. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The nucleic acids may include a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

A construct or vector may be used to introduce the TCR into the recipient cell. Exemplary constructs are described herein. Polynucleotides encoding the alpha and beta chains of the TCR may in a single construct or in separate constructs. The polynucleotides encoding the alpha and beta chains may be operably linked to a promoter, e.g., a heterologous promoter. The heterologous promoter may be a strong promoter, e.g., EF1alpha, CMV, PGK1, Ube, beta actin, CAG promoter, and the like. The heterologous promoter may be a weak promoter. The heterologous promoter may be an inducible promoter. Exemplary inducible promoters include, but are not limited to TRE, NFAT, GAL4, LAC, and the like. Other exemplary inducible expression systems are described in U.S. Pat. Nos. 5,514,578; 6,245,531; 7,091,038 and European Patent No. 0517805, which are incorporated by reference in their entirety.

The construct for introducing the TCR into the recipient cell may also comprise a polynucleotide encoding a signal peptide (signal peptide element). The signal peptide may promote surface trafficking of the introduced TCR. Exemplary signal peptides include, but are not limited to CD4 signal peptide, immunoglobulin signal peptides, where specific examples include GM-CSF and IgG kappa. Such signal peptides are described in Trends Biochem Sci. 2006 October: 31(10):563-71. Epub 2006 Aug. 21; and An, et al. “Construction of a New Anti-CD19 Chimeric Antigen Receptor and the Anti-Leukemia Function Study of the Transduced T-cells.” Oncotarget 7.9 (2016): 10638-10649. PMC. Web. 16 Aug. 2018; which are hereby incorporated by reference.

In some cases, e.g., cases where the alpha and beta chains are expressed from a single construct or open reading frame, or cases wherein a marker gene is included in the construct, the construct may comprise a ribosomal skip sequence. The ribosomal skip sequence may be a 2A peptide, e.g., a P2A or T2A peptide. Exemplary P2A and T2A peptides are described in Scientific Reports volume 7, Article number: 2193 (2017), hereby incorporated by reference in its entirety. In some cases, a FURIN/PACE cleavage site is introduced upstream of the 2A element. FURIN/PACE cleavage sites are described in, e.g., http://www.nuolan.net/substrates.html. The cleavage peptide may also be a factor Xa cleavage site. In cases where the alpha and beta chains are expressed from a single construct or open reading frame, the construct may comprise an internal ribosome entry site (IRES).

The construct may further comprise one or more marker genes. Exemplary marker genes include but are not limited to GFP, luciferase, HA, lacZ. The marker may be a selectable marker, such as an antibiotic resistance marker, a heavy metal resistance marker, or a biocide resistant marker, as is known to those of skill in the art. The marker may be a complementation marker for use in an auxotrophic host. Exemplary complementation markers and auxotrophic hosts are described in Gene. 2001 Jan. 24; 263(1-2):159-69. Such markers may be expressed via an IRES, a frameshift sequence, a 2A peptide linker, a fusion with the TCR, or expressed separately from a separate promoter.

Exemplary vectors or systems for introducing TCRs into recipient cells include, but are not limited to Adeno-associated virus, Adenovirus, Adenovirus+Modified vaccinia, Ankara virus (MVA), Adenovirus+Retrovirus, Adenovirus+Sendai virus, Adenovirus+Vaccinia virus, Alphavirus (VEE) Replicon Vaccine, Antisense oligonucleotide, Bifidobacterium longum, CRISPR-Cas9, E. coli, Flavivirus, Gene gun, Herpesviruses, Herpes simplex virus, Lactococcus lactis, Electroporation, Lentivirus, Lipofection, Listeria monocytogenes, Measles virus, Modified Vaccinia Ankara virus (MVA), mRNA Electroporation, Naked/Plasmid DNA, Naked/Plasmid DNA+Adenovirus, Naked/Plasmid DNA+Modified Vaccinia Ankara virus (MVA), Naked/Plasmid DNA+RNA transfer, Naked/Plasmid DNA+Vaccinia virus, Naked/Plasmid DNA+Vesicular stomatitis virus, Newcastle disease virus, Non-viral, PiggyBac™ (PB) Transposon, nanoparticle-based systems, Poliovirus, Poxvirus, Poxvirus+Vaccinia virus, Retrovirus, RNA transfer, RNA transfer+Naked/Plasmid DNA, RNA virus, Saccharomyces cerevisiae, Salmonella typhimurium, Semliki forest virus, Sendai virus, Shigella dysenteriae, Simian virus, siRNA, Sleeping Beauty transposon, Streptococcus mutans, Vaccinia virus, Venezuelan equine encephalitis virus replicon. Vesicular stomatitis virus, and Vibrio cholera.

In preferred embodiments, the TCR is introduced into the recipient cell via adeno associated virus (AAV), adenovirus, CRISPR-CAS9, herpesvirus, lentivirus, lipofection, mRNA electroporation, PiggyBac™ (PB) Transposon, retrovirus, RNA transfer, or Sleeping Beauty transposon.

In some embodiments, a vector for introducing a TCR into a recipient cell is a viral vector. Exemplary viral vectors include adenoviral vectors, adeno-associated viral (AAV) vectors, lentiviral vectors, herpes viral vectors, retroviral vectors, and the like. Such vectors are described herein.

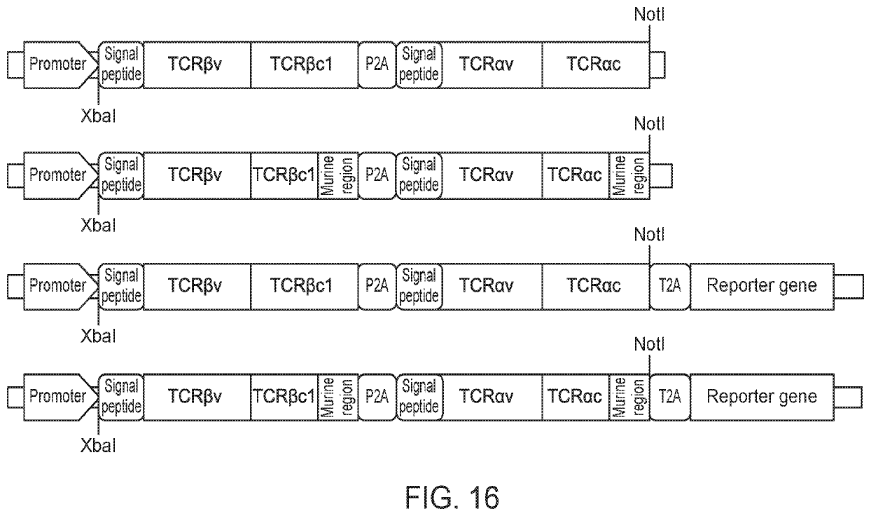

Exemplary embodiments of TCR constructs for introducing a TCR into recipient cells is shown in FIG. 16. In some embodiments, a TCR construct includes, from the 5′-3′ direction, the following polynucleotide sequences: a promoter sequence, a signal peptide sequence, a TCR β variable (TCRβv) sequence, a TCR β constant (TCRβc) sequence, a cleavage peptide (e.g., P2A), a signal peptide sequence, a TCR α variable (TCRαv) sequence, and a TCR α constant (TCRαc) sequence. In some embodiments, the TCRβc and TCRαc sequences of the construct include one or more murine regions, e.g., full murine constant sequences or human→murine amino acid exchanges as described herein. In some embodiments, the construct further includes, 3′ of the TCRαc sequence, a cleavage peptide sequence (e.g., T2A) followed by a reporter gene. In an embodiment, the construct includes, from the 5′-3′ direction, the following polynucleotide sequences: a promoter sequence, a signal peptide sequence, a TCR β variable (TCRβv) sequence, a TCR β constant ((TCRβc) sequence containing one or more murine regions, a cleavage peptide (e.g., P2A), a signal peptide sequence, a TCR α variable (TCRαv) sequence, and a TCR α constant (TCRαc) sequence containing one or more murine regions, a cleavage peptide (e.g., T2A), and a reporter gene.

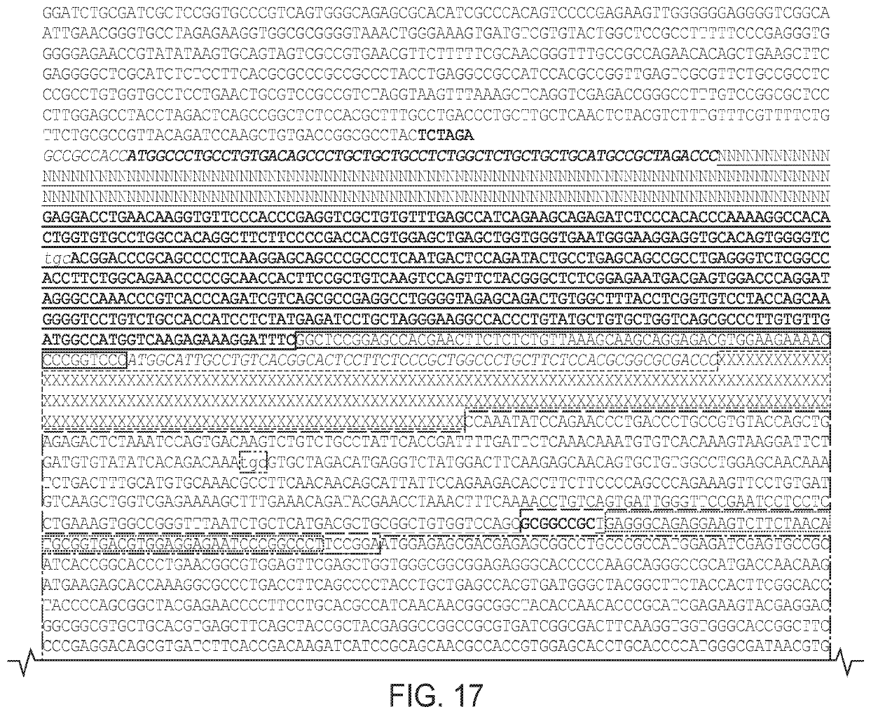

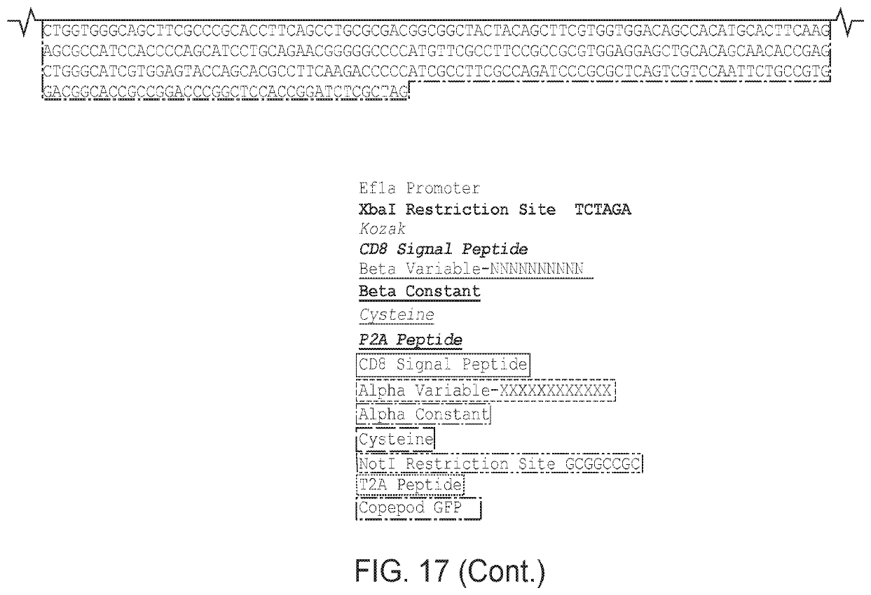

FIG. 17 depicts an exemplary P526 construct backbone nucleotide sequence for cloning TCRs into expression systems for therapy development.

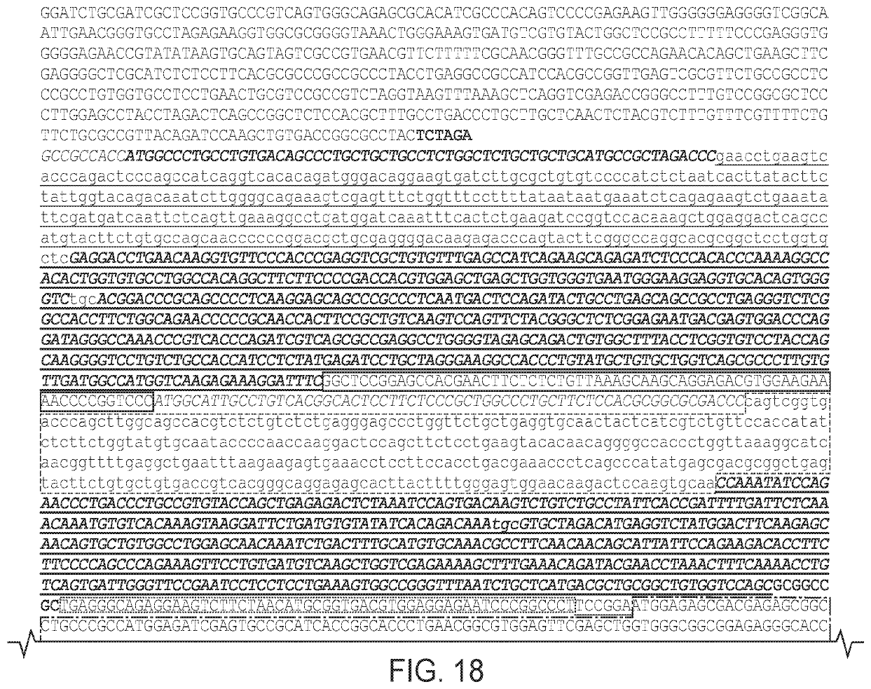

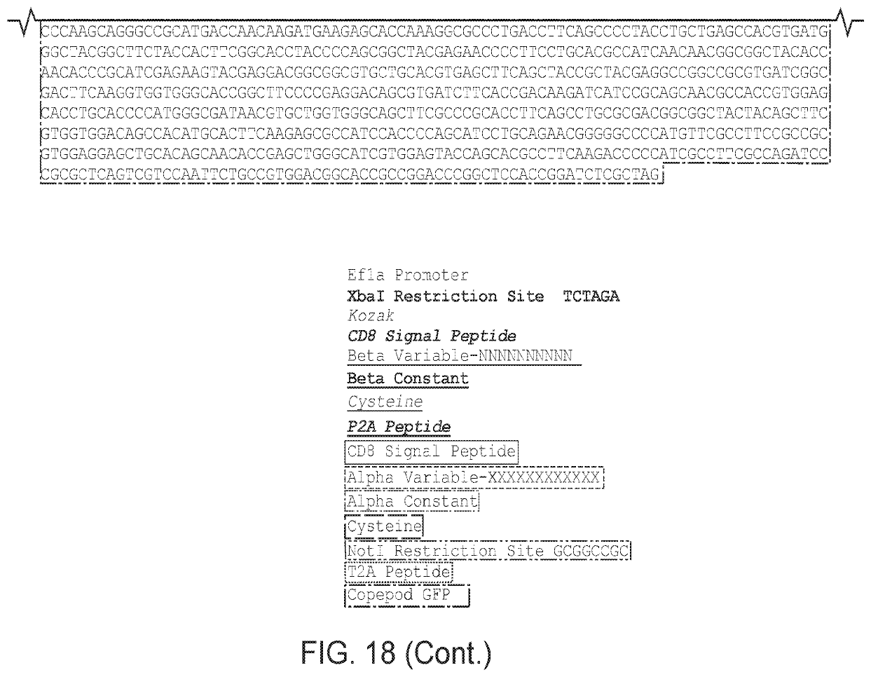

FIG. 18 depicts an exemplary construct sequence for cloning patient neoantigen-specific TCR, clonotype 1 into expression systems for therapy development.

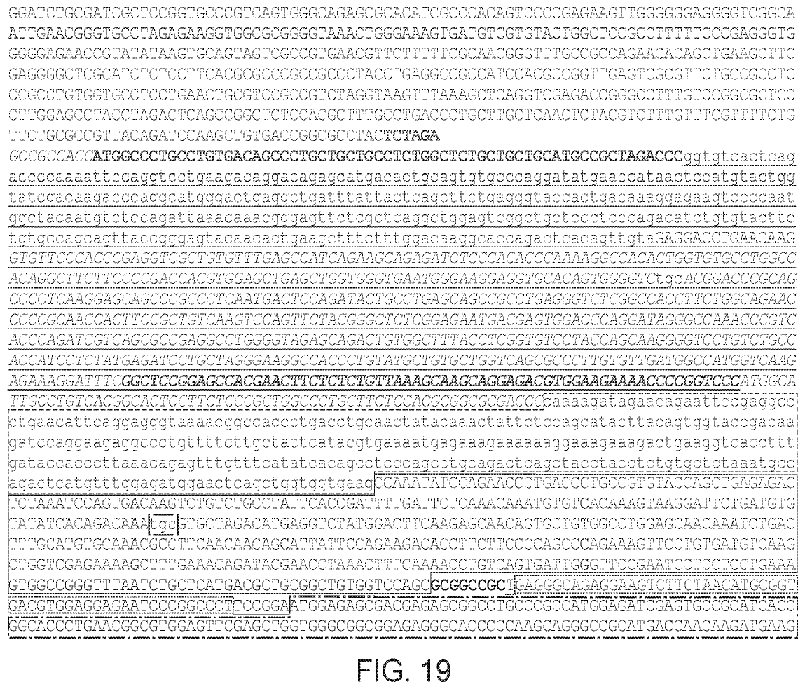

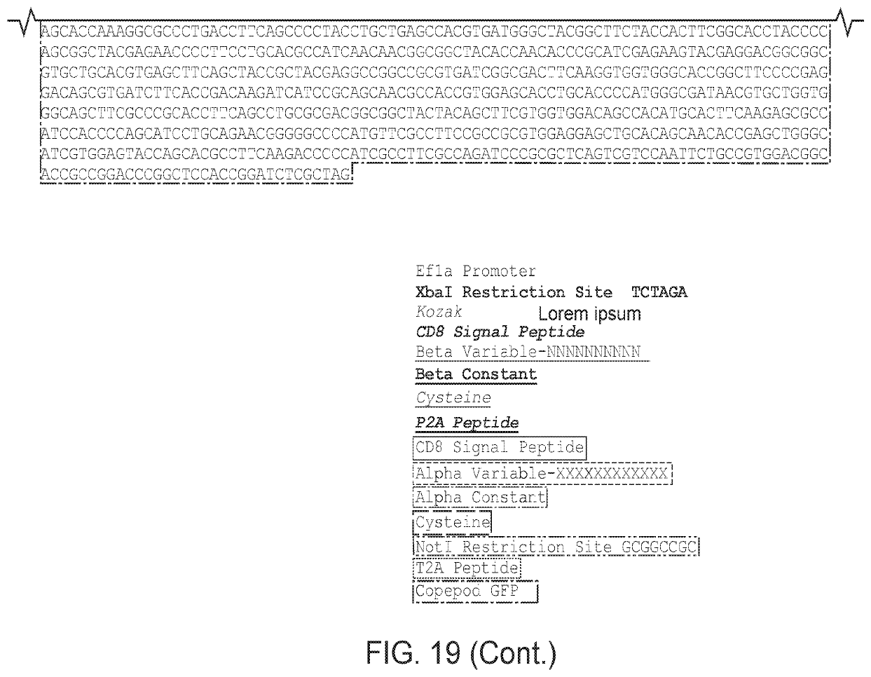

FIG. 19 depicts an exemplary construct sequence for cloning patient neoantigen-specific TCR, clonotype 3 into expression systems for therapy development.

Also provided are isolated nucleic acids encoding TCRs, vectors comprising the nucleic acids, and host cells comprising the vectors and nucleic acids, as well as recombinant techniques for the production of the TCRs.

The nucleic acids may be recombinant. The recombinant nucleic acids may be constructed outside living cells by joining natural or synthetic nucleic acid segments to nucleic acid molecules that can replicate in a living cell, or replication products thereof. For purposes herein, the replication can be in vitro replication or in vivo replication.

For recombinant production of a TCR, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Exemplary vectors or constructs suitable for expressing a TCR, antibody, or antigen binding fragment thereof, include, e.g., the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as AGTlO, AGTI 1, AZapII (Stratagene), AEMBL4, and ANM1 149, are also suitable for expressing a TCR disclosed herein.

### XIX. Treatment Overview Flow Chart

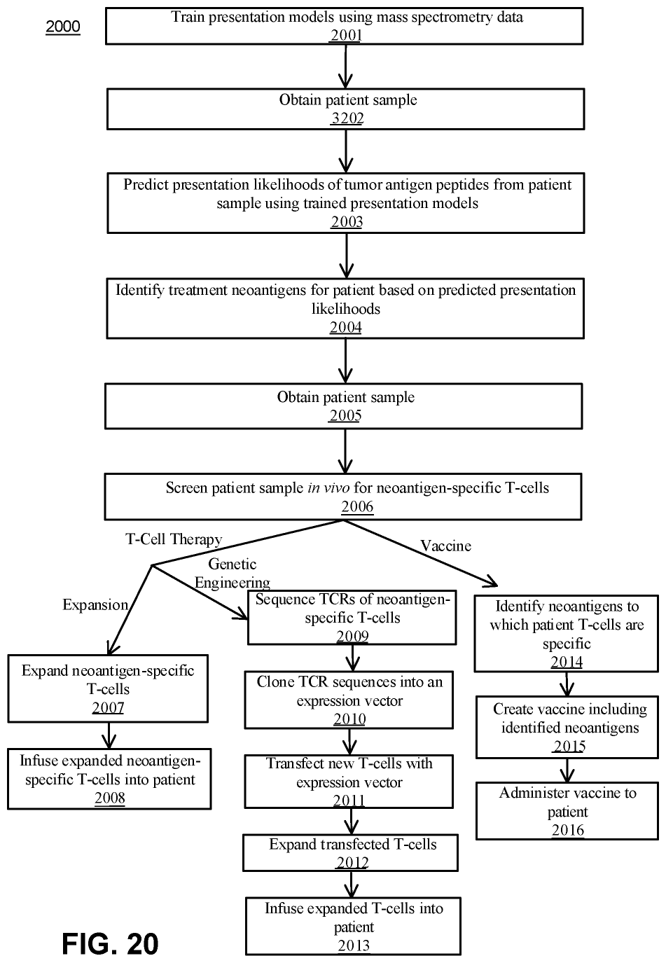

FIG. 20 is a flow chart of a method for providing a customized, neoantigen-specific treatment to a patient, in accordance with an embodiment. In other embodiments, the method may include different and/or additional steps than those shown in FIG. 20. Additionally, steps of the method may be performed in different orders than the order described in conjunction with FIG. 20 in various embodiments.

The presentation models are trained 2001 using mass spectrometry data as described above. A patient sample is obtained 2002. In some embodiments, the patient sample comprises a tumor biopsy and/or the patient's peripheral blood. The patient sample obtained in step 2002 is sequenced to identify data to input into the presentation models to predict the likelihoods that tumor antigen peptides from the patient sample will be presented. Presentation likelihoods of tumor antigen peptides from the patient sample obtained in step 2002 are predicted 2003 using the trained presentation models. Treatment neoantigens are identified 2004 for the patient based on the predicted presentation likelihoods. Next, another patient sample is obtained 2005. The patient sample may comprise the patient's peripheral blood, tumor-infiltrating lymphocytes (TIL), lymph, lymph node cells, and/or any other source of T-cells. The patient sample obtained in step 2005 is screened 2006 in vivo for neoantigen-specific T-cells.

At this point in the treatment process, the patient can either receive T-cell therapy and/or a vaccine treatment. To receive a vaccine treatment, the neoantigens to which the patient's T-cells are specific are identified 2014. Then, a vaccine including the identified neoantigens is created 2015. Finally, the vaccine is administered 2016 to the patient.

To receive T-cell therapy, the neoantigen-specific T-cells undergo expansion and/or new neoantigen-specific T-cells are genetically engineered. To expand the neoantigen-specific T-cells for use in T-cell therapy, the cells are simply expanded 2007 and infused 2008 into the patient.

To genetically engineer new neoantigen-specific T-cells for T-cell therapy, the TCRs of the neoantigen-specific T-cells that were identified in vivo are sequenced 2009. Next, these TCR sequences are cloned 2010 into an expression vector. The expression vector 2010 is then transfected 2011 into new T-cells. The transfected T-cells are 2012 expanded. And finally, the expanded T-cells are infused 2013 into the patient.

A patient may receive both T-cell therapy and vaccine therapy. In one embodiment, the patient first receives vaccine therapy then receives T-cell therapy. One advantage of this approach is that the vaccine therapy may increase the number of tumor-specific T-cells and the number of neoantigens recognized by detectable levels of T-cells.

In another embodiment, a patient may receive T-cell therapy followed by vaccine therapy, wherein the set of epitopes included in the vaccine comprises one or more of the epitopes targeted by the T-cell therapy. One advantage of this approach is that administration of the vaccine may promote expansion and persistence of the therapeutic T-cells.

### XX. Example Computer

FIG. 21 illustrates an example computer 2100 for implementing the entities shown in FIGS. 1 and 3. The computer 2100 includes at least one processor 2102 coupled to a chipset 2104. The chipset 2104 includes a memory controller hub 2120 and an input/output (I/O) controller hub 2122. A memory 2106 and a graphics adapter 2112 are coupled to the memory controller hub 2120, and a display 2118 is coupled to the graphics adapter 2112. A storage device 2108, an input device 2114, and network adapter 2116 are coupled to the I/O controller hub 2122. Other embodiments of the computer 2100 have different architectures.

The storage device 2108 is a non-transitory computer-readable storage medium such as a hard drive, compact disk read-only memory (CD-ROM), DVD, or a solid-state memory device. The memory 2106 holds instructions and data used by the processor 2102. The input interface 2114 is a touch-screen interface, a mouse, track ball, or other type of pointing device, a keyboard, or some combination thereof, and is used to input data into the computer 2100. In some embodiments, the computer 2100 may be configured to receive input (e.g., commands) from the input interface 2114 via gestures from the user. The graphics adapter 2112 displays images and other information on the display 2118. The network adapter 2116 couples the computer 2100 to one or more computer networks.

The computer 2100 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 2108, loaded into the memory 2106, and executed by the processor 2102.

The types of computers 2100 used by the entities of FIG. 1 can vary depending upon the embodiment and the processing power required by the entity. For example, the presentation identification system 160 can run in a single computer 2100 or multiple computers 2100 communicating with each other through a network such as in a server farm. The computers 2100 can lack some of the components described above, such as graphics adapters 2112, and displays 2118.

### REFERENCES

1. Desrichard, A., Snyder, A. & Chan. T. A. Cancer Neoantigens and Applications for Immunotherapy. Clin. Cancer Res. Off. J. Am. Assoc. Cancer Res. (2015), doi:10.1158/1078-0432.CCR-14-3175
2. Schumacher. T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J. Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA4 blockade in melanoma. N. Engl. J. Med. 371, 2189-2199 (2014).
6. Carreno. B. M. et al. Cancer immunotherapy. A dendritic cell vaccine increases the breadth and diversity of melanoma neoantigen-specific T-cells. Science 348, 803-808 (2015).
7. Tran, E. et al. Cancer immunotherapy based on mutation-specific CD4+ T-cells in a patient with epithelial cancer. Science 344, 641-645 (2014).
8. Hacohen, N. & Wu, C. J.-Y. U.S. patent application Ser. No. 01/0293637—COMPOSITIONS AND METHODS OF IDENTIFYING TUMOR SPECIFIC NEOANTIGENS. (A1). at <http://appft1.uspto.gov/netacgi/nph-Parser≙Sect1=PTO1&Sect2=HITOFF&d=PG01&p=1&u=/netahtml/PTO/srchnum.html&r=1&f=G&1=50&s1=201 10293637.PGNR.>
9. Lundegaard, C., Hoof, I., Lund. O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Sternberg. M., Pletscher-Frankild. S., Jensen, L. J. & Mann. M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA4 blockade in metastatic melanoma. Science 350, 207-211 (2015).
13. Yoshida. K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. U.S. patent application Ser. No. 01/20208706—OPTIMIZATION OF MULTIGENE ANALYSIS OF TUMOR SAMPLES. (A1). at <http://appft1.uspto.gov/netacgi/nph-Parser?Sect1=PTO1&Sect2=HITOFF&d=PG01&p=1&u=/netahtml/PTO/srchnum.html&r=1&f=G&1=50&s1=20120208706.PGNR.>
17. Target Capture for NextGen Sequencing—IDT. at <http://www.idtdna.com/pages/products/nextgen/target-capture>
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders. C. T. et al. Strelka: accurate somatic small-variant calling from sequenced tumor-normal sample pairs. Bioinforma. Oxf. Engl. 28, 1811-1817 (2012).
22. Cibulskis. K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, e107 (2014).
24. Mose, L. E., Wilkerson, M. D., Hayes. D. N., Perou, C. M. & Parker. J. S. ABRA: improved coding indel detection via assembly-based realignment. Bioinforma. Oxf. Engl. 30, 2813-2815 (2014).
25. Ye, K., Schulz, M. H., Long. Q., Apweiler. R. & Ning, Z. Pindel: a pattern growth approach to detect break points of large deletions and medium sized insertions from paired-end short reads. Bioinforma. Oxf. Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton. G. M. et al. Development and validation of a clinical cancer genomic profiling test based on massively parallel DNA sequencing. Nat. Biotechnol. 31, 1023-1031 (2013).
28. Boegel. S. et al. HLA typing from RNA-Seq sequence reads. Genome Med. 4, 102 (2012).
29. Liu. C. et al. ATHLATES: accurate typing of human leukocyte antigen through exome sequencing. Nucleic Acids Res. 41, e142 (2013).
30. Mayor. N. P. et al. HLA Typing for the Next Generation. PloS One 10, e0127153 (2015).
31. Roy, C. K., Olson, S., Graveley. B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty. L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel. H., Trapnell. C. & Pachter, L. Identification of novel transcripts in annotated genomes using RNA-Seq. Bioinforma. Oxf. Engl. (2011). doi:10.1093/bioinformatics/btr355
36. Vitting-Seerup. K., Porse. B. T., Sandelin, A. & Waage, J. spliceR: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy. J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl. P. T. & Huber, W. HTSeq—a Python framework to work with high-throughput sequencing data. Bioinforma. Oxf. Engl. 31, 166-169 (2015).
40. Furney, S. J. et al. SF3B1 mutations are associated with alternative splicing in uveal melanoma. Cancer Discov. (2013). doi:10.1158/2159-8290.CD-13-0330
41. Zhou, Q. et al. A chemical genetics approach for the functional assessment of novel cancer genes. Cancer Res. (2015). doi:10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA CoMPASS: a dual approach for pathogen and host transcriptome analysis of RNA-seq datasets. PloS One 9, e89445 (2014).
45. Andreatta, M. & Nielsen, M. Gapped sequence alignment using artificial neural networks: application to the MHC class I system. Bioinforma. Oxf. Engl. (2015). doi:10.1093/bioinformatics/btv639
46. Jorgensen. K. W., Rasmussen, M., Buus, S. & Nielsen. M. NetMHCstab—predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. cytotoxic T-cell epitopes: insights obtained from improved predictions of proteasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M111.011429 (2012).
50. Duan, F. et al. Genomic and bioinformatic profiling of mutational neoepitopes reveals new rules to predict anticancer immunogenicity. J. Exp. Med. 211, 2231-2248 (2014).
51. Janeway's Immunobiology: 9780815345312: Medicine & Health Science Books # Amazon.com. at <http://www.amazon.com/Janeways-Immunobiology-Kenneth-Murphy/dp/0815345313>
52. Calis, J. J. A. et al. Properties of MHC Class I Presented Peptides That Enhance Immunogenicity. PLoS Comput. Biol. 9, e1003266 (2013).
53. Zhang. J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992, 255: 1261-1263.
56. Zarling A L, Polefrone J M, Evans A M, Mikesh L M, Shabanowitz J, Lewis S T, Engelhard V H, Hunt D F, Identification of class I MHC-associated phosphopeptides as targets for cancer immunotherapy. Proc Natl Acad Sci USA. 2006 Oct. 3; 103(40):14889-94.
57. Bassani-Sternberg M, Pletscher-Frankild S, Jensen L J, Mann M, Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
58. Abelin J G, Trantham P D, Penny S A, Patterson A M, Ward S T, Hildebrand W H. Cobbold M, Bai D L, Shabanowitz J, Hunt D F, Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
59. Barnstable C J, Bodmer W F, Brown G, Galfre G. Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng K H. HLA-DR monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4.
62. Eng J K, Hoopmann M R, Jahan T A, Egertson J D, Noble W S, MacCoss M J. A deeper look into Comet-implementation and features. J Am Soc Mass Spectrom. 2015 November; 26(11):1865-74. doi: 10.1007/s13361-015-1179-x. Epub 2015 Jun. 27.
63. Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Käll, John D. Storey, Michael J. MacCoss and William Stafford Noble. Assigning confidence measures to peptides identified by tandem mass spectrometry. Journal of Proteome Research, 7(1):29-34, January 2008
65. Lukas Käll, John D. Storey and William Stafford Noble. Nonparametric estimation of posterior error probabilities associated with peptides identified by tandem mass spectrometry. Bioinformatics. 24(16):i42-i48, August 2008
66. Bo Li and C. olin N. Dewey. RSEM: accurate transcript quantification from RNA-Seq data with or without a referenfe genome. BMC Bioinformatics, 12:323, August 2011
67. Hillary Pearson, Tariq Daouda, Diana Paola Granados, Chantal Durette, Eric Bonneil, Mathieu Courcelles. Anja Rodenbrock, Jean-Philippe Laverdure, Caroline Côté, Sylvie Mader, Sébastien Lemieux, Pierre Thibault, and Claude Perreault. MHC class I-associated peptides derive from selective regions of the human genome. The Journal of Clinical Investigation, 2016,
68. Juliane Liepe, Fabio Marino, John Sidney. Anita Jeko. Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
69. Mommen G P., Marino, F., Meiring H D., Poelen, M C., van Gaans-van den Brink, J A., Mohammed S., Heck A J., and van Els C A. Sampling From the Proteome to the Human Leukocyte Antigen-DR (HLA-DR) Ligandome Proceeds Via High Specificity. Mol Cell Proteomics 15(4): 1412-1423, April 2016.
70. Sebastian Kreiter, Mathias Vormehr, Niels van de Roemer, Mustafa Diken, Martin Löwer, Jan Diekmann, Sebastian Boegel, Barbara Schrörs, Fulvia Vascotto, John C. Castle, Arbel D. Tadmor, Stephen P. Schoenberger, Christoph Huber, Özlem Türeci, and Ugur Sahin. Mutant MHC class II epitopes drive therapeutic immune responses to caner. Nature 520, 692-6%, April 2015.
71. Tran E., Turcotte S., Gros A., Robbins P. F., Lu Y. C., Dudley M. E., Wunderlich J. R., Somerville R. P., Hogan K., Hinrichs C. S., Parkhurst M. R., Yang J.C., Rosenberg S.A. Cancer immunotherapy based on mutation-specific CD4+ T-cells in a patient with epithelial cancer. Science 344(6184) 641-645, May 2014.
72. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
73. Nielsen, M., Lund, O. NN-align. An artificial neural network-based alignment algorithm for MHC class II peptide binding prediction. BMC Bioinformatics 10:296, September 2009.
74. Nielsen, M., Lundegaard, C., Lund, O. Prediction of MHC class II binding affinity using SMM-align, a novel stabilization matrix alignment method. BMC Bioinformatics 8:238, July 2007.
75. Zhang, J., et al. PEAKS DB: de novo sequencing assisted database search for sensitive and accurate peptide identification. Molecular & Cellular Proteomics. 11(4):1-8. Jan. 2, 2012.
76. Snyder, A. et al. Genetic basis for clinical response to CTLA-4 blockade in melanoma. N. Engl. J. Med. 371, 2189-2199 (2014).
77. Rizvi, N. A. et al. Cancer immunology. Mutational landscape determines sensitivity to PD-1 blockade in non-small cell lung cancer. Science 348, 124-128 (2015).
78. Gubin, M. M., Artyomov, M. N., Mardis, E. R. & Schreiber, R. D. Tumor neoantigens: building a framework for personalized cancer immunotherapy. J. Clin. Invest. 125, 3413-3421 (2015).
79. Schumacher, T. N. & Schreiber, R. D. Neoantigens in cancer immunotherapy. Science 348, 69-74 (2015).
80. Carreno. B. M. et al. Cancer immunotherapy. A dendritic cell vaccine increases the breadth and diversity of melanoma neoantigen-specific T-cells. Science 348, 803-808 (2015).
81. Ott, P. A. et al. An immunogenic personal neoantigen vaccine for patients with melanoma. Nature 547, 217-221 (2017).
82. Sahin. U. et al. Personalized RNA mutanome vaccines mobilize poly-specific therapeutic immunity against cancer. Nature 547, 222-226 (2017).
83. Tran, E. et al. T-Cell Transfer Therapy Targeting Mutant KRAS in Cancer. N. Engl. J. Med. 375, 2255-2262 (2016).
84. Gros, A. et al. Prospective identification of neoantigen-specific lymphocytes in the peripheral blood of melanoma patients. Nat. Med. 22, 433-438 (2016).
85. The problem with neoantigen prediction. Nat. Biotechnol. 35, 97-97 (2017).
86. Vitiello. A. & Zanetti, M. Neoantigen prediction and the need for validation. Nat. Biotechnol. 35, 815-817 (2017).
87. Bassani-Sternberg. M., Pletscher-Frankild. S., Jensen, L. J. & Mann. M. Mass spectrometry of human leukocyte antigen class I peptidomes reveals strong effects of protein abundance and turnover on antigen presentation. Mol. Cell. Proteomics MCP 14, 658-673 (2015).
88. Vita. R. et al. The immune epitope database (IEDB) 3.0. Nucleic Acids Res. 43, D405-412 (2015).
89. Andreatta, M. & Nielsen, M. Gapped sequence alignment using artificial neural networks: application to the MHC class I system. Bioinforma. Oxf. Engl. 32, 511-517 (2016).
90. O'Donnell, T. J. et al. MHCflurry: Open-Source Class I MHC Binding Affinity Prediction. Cell Syst. (2018). doi:10.1016/j.cels.2018.05.014
91. Bassani-Sternberg. M. et al. Direct identification of clinically relevant neoepitopes presented on native human melanoma tissue by mass spectrometry. Nat. Commun. 7, 13404 (2016).
92. Abelin, J. G. et al. Mass Spectrometry Profiling of HLA-Associated Peptidomes in Mono-allelic Cells Enables More Accurate Epitope Prediction. Immunity 46, 315-326 (2017).
93. Yadav. M. et al. Predicting immunogenic tumour mutations by combining mass spectrometry and exome sequencing. Nature 515, 572-576 (2014).
94. Stranzl. T., Larsen, M. V., Lundegaard, C. & Nielsen, M. NetCTLpan: pan-specific MHC class I pathway epitope predictions. Immunogenetics 62, 357-368 (2010).
95. Bentzen, A. K. et al. Large-scale detection of antigen-specific T-cells using peptide-MHC-I multimers labeled with DNA barcodes. Nat. Biotechnol. 34, 1037-1045 (2016).
96. Tran. E. et al. Immunogenicity of somatic mutations in human gastrointestinal cancers. Science 350, 1387-1390 (2015).
97. Stronen, E. et al. Targeting of cancer neoantigens with donor-derived T-cell receptor repertoires. Science 352, 1337-1341 (2016).
98. Trolle. T. et al. The Length Distribution of Class I-Restricted T-cell Epitopes Is Determined by Both Peptide Supply and MHC Allele-Specific Binding Preference. J. Immunol. Baltim. Md. 1950 196, 1480-1487 (2016).
99. Di Marco, M. et al. Unveiling the Peptide Motifs of HLA-C and HLA-G from Naturally Presented Peptides and Generation of Binding Prediction Matrices. J. Immunol. Baltim. Md. 1950 199, 2639-2651 (2017).
100. Goodfellow. I., Bengio, Y. & Courville, A. Deep Learning. (MIT Press, 2016).
101. Sette. A. et al. The relationship between class I binding affinity and immunogenicity of potential cytotoxic T-cell epitopes. J. Immunol. Baltim. Md. 1950 153, 5586-5592 (1994).
102. Fortier, M.-H. et al. The MHC class I peptide repertoire is molded by the transcriptome. J. Exp. Med. 205, 595-610 (2008).
103. Pearson. H. et al. MHC class I-associated peptides derive from selective regions of the human genome. J. Clin. Invest. 126, 4690-4701 (2016).
104. Bassani-Sternberg, M. et al. Deciphering HLA-I motifs across HLA peptidomes improves neoantigen predictions and identifies allostery regulating HLA specificity. PLoS Comput. Biol. 13, e1005725 (2017).
105. Andreatta, M., Lund, O. & Nielsen. M. Simultaneous alignment and clustering of peptide data using a Gibbs sampling approach. Bioinforma. Oxf. Engl. 29, 8-14 (2013).
106. Andreatta, M., Alvarez, B. & Nielsen, M. GibbsCluster: unsupervised clustering and alignment of peptide sequences. Nucleic Acids Res. (2017). doi:10.1093/nar/gkx248
107. Gros. A. et al. Prospective identification of neoantigen-specific lymphocytes in the peripheral blood of melanoma patients. Nat. Med. 22, 433-438 (2016).
108. Zacharakis, N. et al. Immune recognition of somatic mutations leading to complete durable regression in metastatic breast cancer. Nat. Med. 24, 724-730 (2018).
109. Chudley, L. et al. Harmonisation of short-term in vitro culture for the expansion of antigen-specific CD8+ T-cells with detection by ELISPOT and HLA-multimer staining. Cancer Immunol. Immunother. 63, 1199-1211 (2014).
110. Van Allen, E. M. et al. Genomic correlates of response to CTLA-4 blockade in metastatic melanoma. Science 350, 207-211 (2015).
111. Anagnostou. V. et al. Evolution of Neoantigen Landscape during Immune Checkpoint Blockade in Non-Small Cell Lung Cancer. Cancer Discov. 7, 264-276 (2017).
112. Carreno. B. M. et al. Cancer immunotherapy. A dendritic cell vaccine increases the breadth and diversity of melanoma neoantigen-specific T-cells. Science 348, 803-808 (2015).
113. Stevanović. S. et al. Landscape of immunogenic tumor antigens in successful immunotherapy of virally induced epithelial cancer. Science 356, 200-205 (2017).
114. Pasetto, A. et al. Tumor- and Neoantigen-Reactive T-cell Receptors Can Be Identified Based on Their Frequency in Fresh Tumor. Cancer Immunol. Res. 4, 734-743 (2016).
115. Gillette, M. A. & Carr, S. A. Quantitative analysis of peptides and proteins in biomedicine by targeted mass spectrometry. Nat. Methods 10, 28-34 (2013).
116. Boegel. S., Löwer. M., Bukur, T., Sahin. U. & Castle, J. C. A catalog of HLA type, HLA expression, and neo-epitope candidates in human cancer cell lines. Oncoimmunology 3. e954893 (2014).
117. Johnson, D. B. et al. Melanoma-specific MHC-II expression represents a tumour-autonomous phenotype and predicts response to anti-PD-I/PD-L1 therapy. Nat. Commun. 7, 10582 (2016).
118. Robbins, P. F. et al. A Pilot Trial Using Lymphocytes Genetically Engineered with an NY-ESO-1-Reactive T-cell Receptor: Long-term Follow-up and Correlates with Response. Clin. Cancer Res. 21, 1019-1027 (2015).
119. Snyder, A. et al. Genetic basis for clinical response to CTLA-4 blockade in melanoma. N. Engl. J. Med. 371, 2189-2199 (2014).
120. Calis. J. J. A. et al. Properties of MHC class I presented peptides that enhance immunogenicity. PLoS Comput. Biol. 9. e1003266 (2013).
121. Duan. F. et al. Genomic and bioinformatic profiling of mutational neoepitopes reveals new rules to predict anticancer immunogenicity. J. Exp. Med. 211, 2231-2248 (2014).
122. Glanville, J. et al. Identifying specificity groups in the T-cell receptor repertoire. Nature 547, 94-98(2017).
123. Dash, P. et al. Quantifiable predictive features define epitope-specific T-cell receptor repertoires. Nature 547, 89-93 (2017).
124. Hunt, D. F. et al. Pillars article: Characterization of peptides bound to the class 1 MHC molecule HLA-A2.1 by mass spectrometry. Science 1992, 255: 1261-1263. J. Immunol. Baltim. Md. 1950 179, 2669-2671 (2007).
125. Zarling. A. L. et al. Identification of class I MHC-associated phosphopeptides as targets for cancer immunotherapy. Proc. Natl. Acad. Sci. U.S.A 103, 14889-14894 (2006).
126. Abelin, J. G. et al. Complementary IMAC enrichment methods for HLA-associated phosphopeptide identification by mass spectrometry. Nat. Protoc. 10, 1308-1318 (2015).
127. Barnstable. C. J. et al. Production of monoclonal antibodies to group A erythrocytes, HLA and other human cell surface antigens-new tools for genetic analysis. Cell 14, 9-20 (1978).
128. Eng, J. K., Jahan, T. A. & Hoopmann. M. R. Comet: an open-source MS/MS sequence database search tool. Proteomics 13, 22-24 (2013).
129. Eng. J. K. et al. A deeper look into Comet-implementation and features. J. Am. Soc. Mass Spectrom. 26, 1865-1874 (2015).
130. Käll, L., Storey, J. D., MacCoss. M. J. & Noble. W. S. Assigning significance to peptides identified by tandem mass spectrometry using decoy databases. J. Proteome Res. 7, 29-34 (2008).
131. Käll, L., Storey. J. D. & Noble. W. S. Non-parametric estimation of posterior error probabilities associated with peptides identified by tandem mass spectrometry. Bioinforma. Oxf. Engl. 24, i42-48 (2008).
132. Käll, L., Canterbury, J. D., Weston, J., Noble. W. S. & MacCoss, M. J. Semi-supervised learning for peptide identification from shotgun proteomics datasets. Nat. Methods 4, 923-925 (2007).
133. Li, B. & Dewey, C. N. RSEM: accurate transcript quantification from RNA-Seq data with or without a reference genome. BMC Bioinformatics 12, 323 (2011).
134. Chollet, F. & others. Keras. (2015).
135. Bastien, F. et al. Understanding the difficulty of training deep feedforward neural networks. Proc. Thirteen. Int. Conf. Artif. Intell. Stat. 249-256 (2010).
136. Glorot. X. & Bengio, Y. Understanding the difficulty of training deep feedforward neural networks, in Proceedings of the Thirteenth International Conference on Artificial Intelligence and Statistics 249-256 (2010).
137. Kingma, D. & Ba, J. Adam: A method for stochastic optimization ArXiv Prepr. ArXiv14126980 (2014).
138. Schneider. T. D. & Stephens, R. M. Sequence logos: a new way to display consensus sequences. Nucleic Acids Res. 18, 6097-6100 (1990).
139. Rubinsteyn, A., O'Donnell, T., Damaraju, N. & Hammerbacher, J. Predicting Peptide-MHC Binding Affinities With Imputed Training Data. biorxiv (2016). doi:https://doi.org/10.1101/054775
140. Tran. E. et al. Immunogenicity of somatic mutations in human gastrointestinal cancers. Science 350, 1387-1390 (2015).
141. Stronen, E. et al. Targeting of cancer neoantigens with donor-derived T-cell receptor repertoires. Science 352, 1337-1341 (2016).
142. Janetzki. S., Cox, J. H., Oden, N. & Ferrari, G. Standardization and validation issues of the ELISPOT assay. Methods Mol. Biol. Clifton N.J. 302, 51-86 (2005).
143. Janetzki, S. et al. Guidelines for the automated evaluation of Elispot assays. Nat. Protoc. 10, 1098-1115 (2015).
144. Li, H. & Durbin. R. Fast and accurate short read alignment with Burrows-Wheeler transform. Bioinforma. Oxf. Engl. 25, 1754-1760 (2009).
145. DePristo. M. A. et al. A framework for variation discovery and genotyping using next-generation DNA sequencing data. Nat. Genet. 43, 491-498 (2011).
146. Garrison, E. & Marth, G. Haplotype-based variant detection from short-read sequencing. arXiv (2012).
147. Cingolani, P. et al. A program for annotating and predicting the effects of single nucleotide polymorphisms. SnpEff: SNPs in the genome of Drosophila melanogaster strain w1118; iso-2; iso-3. Fly (Austin) 6, 80-92 (2012).
148. Szolek, A. et al. OptiType: precision HLA typing from next-generation sequencing data. Bioinforma. Oxf. Engl. 30, 3310-3316 (2014).
149. Cibulskis, K. et al. Sensitive detection of somatic point mutations in impure and heterogeneous cancer samples. Nat. Biotechnol. 31, 213-219 (2013).
150. Scholz, E. M. et al. Human Leukocyte Antigen (HLA)-DRB1*15:01 and HLA-DRB5*01:01 Present Complementary Peptide Repertoires. Front. Immunol. 8, 984 (2017).
151. Ooi. J. D. et al. Dominant protection from HLA-linked autoimmunity by antigen-specific regulatory T-cells. Nature 545, 243-247 (2017).
152. Karosiene, E. et al. NetMHCIIpan-3.0, a common pan-specific MHC class II prediction method including all three human MHC class II isotypes, HLA-DR, HLA-DP and HLA-DQ. Immunogenetics 65, 711-724 (2013).
153. Dudley M E, Gross C A, Langhan M M, et al. CD8+ enriched “young” tumor infiltrating lymphocytes can mediate regression of metastatic melanoma. Clinical cancer research: an official journal of the American Association for Cancer Research. 2010; 16(24):6122-6131. doi:10.1158/1078-0432.CCR-10-1297.
154. Dudley M E, Wunderlich J R, Shelton T E, Even J, Rosenberg S A. Generation of Tumor-Infiltrating Lymphocyte Cultures for Use in Adoptive Transfer Therapy for Melanoma Patients. Journal of immunotherapy (Hagerstown, Md.: 1997). 2003; 26(4):332-342.
155. Cohen C J, Gartner J J, Horovitz-Fried M, et al. Isolation of neoantigen-specific T cells from tumor and peripheral lymphocytes. The Journal of Clinical Investigation. 2015; 125(10):3981-3991. doi:10.1172/JCI82416.
156. Kelderman. S., Heemskerk, B, Fanchi, L., Philips, D., Toebes, M., Kvistborg. P, Buuren, M. M., Rooij, N., Michels, S., Genneroth, L., Haanen, J. B. and Schumacher. N. M. (2016), Antigen-specific TIL therapy for melanoma: A flexible platform for personahied cancer immunotherapy. Eur. J. Immunol., 46: 1351-1360. doi:10.1002/eji.201545849.
157. Hall M, Liu H, Malafa M. et al. Expansion of tumor-infiltrating lymphocytes (TIL) from human pancreatic tumors. Journal for Immunotherapy of Cancer. 2016:4:61. doi.10.1186/s40425-016-0164-7.
158. Briggs A. Goldfless S. Timberlake S. et al. Tumor-infiltrating immune repertoires captured by single-cell barcoding in emulsion. bioRxiv. 2017. doi.org/10.1101/134841.
159. US Patent Application No. 20160244825A1.
160. Szegedy, C., Liu. W., Jia, Y., Sermanet, P., Reed, S., Anguelov, D., . . . Arbor, A. (2014). Going Deeper with Convolutions. 1-9. ArXiv 1409.4842. doi.org/10.1109/CVPR.2015.7298594, P., Reed, S., Anguelov, D., . . . Arbor, A. (2014). Going Deeper with Convolutions. 1-9.
161. Maiers, M, et al. “High-resolution HLA alleles and haplotypes in the United States population.” Human Immunology, vol. 68, no. 9, 24 May 2007, pp. 779-88, doi:https://www.sciencedirect.com/science/article/abs/pii/S198885907000948?via%3Dihub.
162. Mak, Tak W., et al. Primer to the Immune Response. 2nd ed., Elsevier Inc., 2014, pp. 143-59, www.sciencedirect.com/science/article/pii/B97801238524580000066?via%3Dihub.
163. Parkhurst, M. R., Robbins, P. F., Tran, E., Prickett, T. D., Gartner, J. J., Jia, L., . . . Rosenberg, S. A. (2019). Unique Neoantigens Arise from Somatic Mutations in Patients with Gastrointestinal Cancers. Cancer Discovery, 9(8), 1022-1035. https.//doi.org/10.1158/2159-8290.cd-18-1494

1. A method for identifying one or more T-cells that are antigen-specific for at least one neoantigen from one or more tumor cells of a subject that are likely to be presented by one or more class II MHC alleles on a surface of the tumor cells, the method comprising the steps of:
obtaining at least one of exome, transcriptome, or whole genome nucleotide sequencing data from the tumor cells and normal cells of the subject, wherein the nucleotide sequencing data is used to obtain data representing peptide sequences of each of a set of neoantigens identified by comparing the nucleotide sequencing data from the tumor cells and the nucleotide sequencing data from the normal cells, wherein the peptide sequence of each neoantigen comprises at least one alteration that makes it distinct from the corresponding wild-type peptide sequence identified from the normal cells of the subject;
encoding the peptide sequences of each of the neoantigens into a corresponding numerical vector, each numerical vector including information regarding a plurality of amino acids that make up the peptide sequence and a set of positions of the amino acids in the peptide sequence;
inputting the numerical vectors, using a computer processor, into a machine-learned presentation model to generate a set of presentation likelihoods for the set of neoantigens, each presentation likelihood in the set representing the likelihood that a corresponding neoantigen is presented by the one or more class II MHC alleles on the surface of the tumor cells of the subject, the machine-learned presentation model comprising:
a plurality of parameters identified at least based on a training data set comprising:
for each sample in a plurality of samples, a label obtained by mass spectrometry measuring presence of peptides bound to at least one class II MHC allele in a set of class II MHC alleles identified as present in the sample; and
for each of the samples, training peptide sequences encoded as numerical vectors including information regarding a plurality of amino acids that make up the peptides and a set of positions of the amino acids in the peptides;
a function representing a relation between the numerical vector received as input and the presentation likelihood generated as output based on the numerical vector and the parameters;
selecting a subset of the set of neoantigens based on the set of presentation likelihoods to generate a set of selected neoantigens;
identifying one or more T-cells that are antigen-specific for at least one of the neoantigens in the subset; and
returning the one or more identified T-cells.

2. The method of claim 1, wherein inputting the numerical vector into the machine-learned presentation model comprises:
applying the machine-learned presentation model to the peptide sequence of the neoantigen to generate a dependency score for each of the one or more class II MHC alleles indicating whether the class II MHC allele will present the neoantigen based on the particular amino acids at the particular positions of the peptide sequence.

3. The method of claim 2, wherein inputting the numerical vector into the machine-learned presentation model further comprises:
transforming the dependency scores to generate a corresponding per-allele likelihood for each class II MHC allele indicating a likelihood that the corresponding class II MHC allele will present the corresponding neoantigen; and
combining the per-allele likelihoods to generate the presentation likelihood of the neoantigen.

4. The method of claim 3, wherein the transforming the dependency scores models the presentation of the neoantigen as mutually exclusive across the one or more class II MHC alleles.

5. The method of claim 2, wherein inputting the numerical vector into the machine-learned presentation model further comprises:
transforming a combination of the dependency scores to generate the presentation likelihood, wherein transforming the combination of the dependency scores models the presentation of the neoantigen as interfering between the one or more class II MHC alleles.

6. The method of any one of claims 2-5, wherein the set of presentation likelihoods are further identified by at least one or more allele noninteracting features, and further comprising:
applying the machine-learned presentation model to the allele noninteracting features to generate a dependency score for the allele noninteracting features indicating whether the peptide sequence of the corresponding neoantigen will be presented based on the allele noninteracting features.

7. The method of claim 6, further comprising:
combining the dependency score for each class II MHC allele in the one or more class II MHC alleles with the dependency score for the allele noninteracting features:
transforming the combined dependency scores for each class II MHC allele to generate a per-allele likelihood for each class II MHC allele indicating a likelihood that the corresponding class II MHC allele will present the corresponding neoantigen; and
combining the per-allele likelihoods to generate the presentation likelihood.

8. The method of claim 6, further comprising:
combining the dependency scores for each of the class II MHC alleles and the dependency score for the allele noninteracting features; and transforming the combined dependency scores to generate the presentation likelihood.

9. The method of any one of claims 1-8, wherein the one or more class II MHC alleles include two or more different class II MHC alleles.

10. The method of any one of claims 1-9, wherein the at least one class II MHC allele includes two or more different types of class II MHC alleles.

11. The method of any one of claims 1-10 wherein the peptide sequences comprise peptide sequences having lengths other than 9 amino acids.

12. The method of any one of claims 1-11, wherein encoding the peptide sequence comprises encoding the peptide sequence using a one-hot encoding scheme.

13. The method of any one of claims 1-12 wherein the plurality of samples comprise at least one of:
(a) one or more cell lines engineered to express a single class II MHC allele;
(b) one or more cell lines engineered to express a plurality of class II MHC alleles;
(c) one or more human cell lines obtained or derived from a plurality of patients;
(d) fresh or frozen tumor samples obtained from a plurality of patients; and
(c) fresh or frozen tissue samples obtained from a plurality of patients.

14. The method of any one of claims 1-13, wherein the training data set further comprises at least one of:
(a) data associated with peptide-MHC binding affinity measurements for at least one of the peptides; and
(b) data associated with peptide-MHC binding stability measurements for at least one of the peptides.

15. The method of any one of claims 1-14, wherein the set of presentation likelihoods are further identified by at least expression levels of the one or more class II MHC alleles in the subject, as measured by RNA-seq or mass spectrometry.

16. The method of any one of claims 1-15, wherein the set of presentation likelihoods are further identified by features comprising at least one of:
(a) predicted affinity between a neoantigen in the set of neoantigens and the one or more class II MHC alleles; and
(b) predicted stability of the neoantigen encoded peptide-MHC complex.

17. The method of any one of claims 1-16, wherein the set of numerical likelihoods are further identified by features comprising at least one of:
(a) the C-terminal sequences flanking the neoantigen encoded peptide sequence within its source protein sequence; and
(b) the N-terminal sequences flanking the neoantigen encoded peptide sequence within its source protein sequence.

18. The method of any one of claims 1-17, wherein selecting the set of selected neoantigens comprises selecting neoantigens that have an increased likelihood of being presented on the tumor cell surface relative to unselected neoantigens based on the machine-learned presentation model.

19. The method of any one of claims 1-18, wherein selecting the set of selected neoantigens comprises selecting neoantigens that have an increased likelihood of being capable of inducing a tumor-specific immune response in the subject relative to unselected neoantigens based on the machine-learned presentation model.

20. The method of any one of claims 1-19, wherein selecting the set of selected neoantigens comprises selecting neoantigens that have an increased likelihood of being capable of being presented to naïve T-cells by professional antigen presenting cells (APCs) relative to unselected neoantigens based on the presentation model, optionally wherein the APC is a dendritic cell (DC).

21. The method of any one of claims 1-20, wherein selecting the set of selected neoantigens comprises selecting neoantigens that have a decreased likelihood of being subject to inhibition via central or peripheral tolerance relative to unselected neoantigens based on the machine-learned presentation model.

22. The method of any one of claims 1-21, wherein selecting the set of selected neoantigens comprises selecting neoantigens that have a decreased likelihood of being capable of inducing an autoimmune response to normal tissue in the subject relative to unselected neoantigens based on the machine-learned presentation model.

23. The method of any one of claims 1-22, wherein the one or more tumor cells are selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T-cell lymphocytic leukemia, non-small cell lung cancer, and small cell lung cancer.

24. The method of any one of claims 1-23, further comprising generating an output for constructing a personalized cancer vaccine from the set of selected neoantigens.

25. The method of claim 24, wherein the output for the personalized cancer vaccine comprises at least one peptide sequence or at least one nucleotide sequence encoding the set of selected neoantigens.

26. The method of any one of claims 1-25, wherein the machine-learned presentation model is a neural network model.

27. The method of claim 26, wherein the neural network model includes a plurality of network models for the class II MHC alleles, each network model assigned to a corresponding class II MHC allele of the class II MHC alleles and including a series of nodes arranged in one or more layers.

28. The method of claim 27, wherein each network model further includes one or more convolutional neural networks, each of the one or more convolutional neural networks including a series of nodes arranged in one or more layers and having a filter of a different size, the filter of each of the one or more convolutional neural networks sized to identify the positions of the amino acids in the peptide sequence of each neoantigen that comprise a binding core or a binding anchor of the peptide sequence.

29. The method of any one of claims 27-28, wherein the neural network model is trained by updating the parameters of the neural network model, and wherein the parameters of at least two network models are jointly updated for at least one training iteration.

30. The method of any one of claims 26-29, wherein the machine-learned presentation model is a deep learning model that includes one or more layers of nodes.

31. The method of any one of claims 1-30, wherein identifying the one or more T-cells comprises co-culturing the one or more T-cells with one or more of the neoantigens in the subset under conditions that expand the one or more T-cells.

32. The method of any one of claims 1-31, wherein identifying the one or more T-cells comprises contacting the one or more T-cells with an MHC multimer comprising one or more of the neoantigens in the subset under conditions that allow binding between the T-cells and the MHC multimer.

33. The method of any one of claims 1-32, further comprising identifying one or more T-cell receptors (TCR) of the one or more identified T-cells.

34. The method of claim 33, wherein identifying the one or more T-cell receptors comprises sequencing the T-cell receptor sequences of the one or more identified T-cells.

35. An isolated T-cell that is antigen-specific for at least one selected neoantigen in the subset of any one of claims 1-34.

36. The method of claim 34, further comprising:
genetically engineering a plurality of T-cells to express at least one of the one or more identified T-cell receptors;
culturing the plurality of T-cells under conditions that expand the plurality of T-cells; and
infusing the expanded T-cells into the subject.

37. The method of claim 36, wherein genetically engineering the plurality of T-cells to express at least one of the one or more identified T-cell receptors comprises:
cloning the T-cell receptor sequences of the one or more identified T-cells into an expression vector; and
transfecting each of the plurality of T-cells with the expression vector.

38. The method of any one of claims 1-37, further comprising:
culturing the one or more identified T-cells under conditions that expand the one or more identified T-cells; and
infusing the expanded T-cells into the subject.
